(19) United States
(12) Patent Application Publication
(10) Pub. No.: US 2015/0299803 A1
(43) Pub. Date: Oct. 22, 2015

(54) Methods of Using Biomarkers for the Treatment of Cancer by Modulation of BCL2 Expression

(71) Applicant: PRONAI THERAPEUTICS, INC., Kalamazoo, MI (US)

(72) Inventors: Wendi Veloso Rodrigueza, Boston, MA (US); Robert T. Streeper, San Antonio, TX (US); Elzbieta Izbicka, San Antonio, TX (US); Mina Patel Sooch, West Bloomfield, MI (US); Michael James Woolliscroft, Ann Arbor, MI (US); Richard Adam Messmann, Brighton, MI (US); Shari Kay Gaylor, Kalamazoo, MI (US)

(21) Appl. No.: 14/440,866

(22) PCT Filed: Nov. 5, 2013
(86) PCT No.: PCT/US2013/068586

§ 371 Date: May 5, 2015

Related U.S. Application Data
(60) Provisional application No. 61/722,764, filed on Nov. 5, 2012.

(51) Int. Cl. C12Q 1/68; G01N 33/50; C12N 15/113; G01N 33/574

(52) U.S. Cl. CPC C12Q 1/6886; G01N 33/5748; G01N 33/5011; C12N 15/1135; C12Q 2600/158; C12Q 2600/136; C12Q 2600/106; G01N 2800/52; G01N 2333/82

(57) ABSTRACT

The present invention relates to cancer therapies and methods of using the same. In particular, the present invention provides methods of monitoring and improving the administration of cancer therapies, wherein the cancer is mediated by the BCL2 oncogene, via markers of disease identification, disease progression, drug resistance, and/or treatment efficacy.

### TECHNICAL FIELD OF THE INVENTION

The present invention relates to cancer therapies and methods of using the same. In particular, the present invention provides methods of monitoring and improving the administration of cancer therapies, wherein the cancer is mediated by the BCL2 oncogene, via markers of disease identification, disease progression, drug resistance, and/or treatment efficacy.

### PRIORITY CLAIM

This application claims priority to U.S. Application Ser. No. 61/722,764, filed Nov. 5, 2012. The entire contents of the aforementioned application are incorporated herein by reference.

### SEQUENCE LISTING

This application incorporates by reference in its entirety the Sequence Listing entitled “Sequence_{—}2012.txt” (698 KB) which was created Nov. 5, 2012 and filed herewith on Nov. 5, 2012.

### BACKGROUND OF THE INVENTION

Oncogenes have become the central concept in understanding cancer biology and may provide valuable targets for therapeutic drugs. In many types of human tumors, including lymphomas and leukemias, oncogenes are overexpressed, and may be associated with tumorigenicity (Tsujimoto et al., Science 228:1440-1443 (1985)). For instance, high levels of expression of the human BCL2 gene have been found in all lymphomas with a t(14; 18) chromosomal translocations including most follicular B cell lymphomas and many large cell non-Hodgkin's lymphomas. High levels of BCL2 gene expression have also been found in certain leukemias that do not have a t(14; 18) chromosomal translation, including most cases of chronic lymphocytic leukemia acute, many lymphocytic leukemias of the pre-B cell type, neuroblastomas, nasopharyngeal carcinomas, and many adenocarcinomas of the prostate, breast and colon. (Reed et al., Cancer Res. 51:6529 [1991]; Yunis et al., New England J. Med. 320:1047; Campos et al., Blood 81:3091-3096 [1993]; McDonnell et al., Cancer Res. 52:6940-6944 [1992]; Lu et al., Int. J Cancer 53:29-35 [1993]; Bonner et al., Lab Invest. 68:43A [1993]; Klamper et al., PNAS 93: 14059-14064 [1996]; Paz-Priel et al., Mol Cancer Res 3:585-596 [2005]. Other important oncogenes include TGF-α, c-ki-ras, ras, Her-2 and c-myc.

BCL2 is a classical cancer target because it is upregulated in cancer cells, but not normal cells. The BCL2 protein is known to drive hematological cancers such as follicular lymphoma (FL), diffuse-large B-cell lymphoma (DLCL) and chronic lymphocytic leukemia (CLL). Further, BCL2-upregulation drives tumor cell resistance to cytotoxic insult and programmed cell death in many solid cancer types thereby making them resistant to current therapies.

The deregulation of apoptosis is a defining characteristic of malignant cells and it is a process in which the overexpression of the BCL2 protein plays a key role. The elevated BCL2/anti-apoptotic phenotype can contribute to the chemoresistance of a broad variety of tumors including diffuse large B-cell lymphoma and many solid tumors. Given this biological importance, BCL2 is a prime target for drug discovery. Previous approaches to modulating BCL2 have included RNA-targeted antisense oligonucleotides, small molecule protein inhibitors and others.

Prior work done with BCL2-targeting oligonucleotides showed that interacting directly with DNA can silence the gene thereby killing cancer cells and have therapeutic value. The oligomer PNT100 targets an un-transcribed region of the promoter of BCL2 and therefore does not act via translational suppression of BCL2 protein synthesis. PNT100, a 24-base DNA oligonucleotide sequence appears to bind to its promoter target whether or not the t(14,18) translocation event known to drive certain lymphomas has involved the BCL2 gene.

The present invention discloses biomarkers useful to identify cancers that respond to the modulation of the BCL2 gene, to evaluate the expression of associated biomarkers, and refine the administration of said therapies in patients.

### SUMMARY OF THE INVENTION

Aspects of the present invention include methods to utilize biomarkers to define patients with cancers that respond to administration of the said test compound for the treatment of a BCL2 mediated cancer in a subject having cancer comprising: obtaining a biological sample from the subject before administration or subsequent to the administration of said test compound; detecting the levels of one or more of a biomarker in the biological sample, wherein the biomarker is selected from the group consisting of, but not limited to: Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32; q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof; comparing the levels of said one or more biomarkers to a measurable threshold for each biomarker, wherein each measurable threshold for each biomarker is based on the level of that biomarker present in a prior biological sample obtained prior to the obtaining of the post-administration biological sample or by some other clinical standard; determining that the dose of reference level, wherein BCL2 is modulated when the levels in the post-administration biological sample differ from the measurable determined threshold or that a patient derives clinical benefit (e.g., tumor shrinkage, improvement in quality of life, improvement in progression free or overall survival).

Aspects of the present invention include a method of determining the down-regulation of the expression of BCL2 after administration of a test compound for the treatment of a BCL2 mediated cancer in a subject having cancer comprising: administering the test compound; obtaining a biological sample from the subject, subsequent to the administration of said test compound; detecting the levels of one or more of a biomarker in the biological sample, wherein the biomarker is selected from the group consisting of but not limited to: Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof; comparing the levels of said one or more biomarkers to a measurable threshold for each biomarker, wherein each measurable threshold for each biomarker is based on the level of that biomarker present in a prior biological sample obtained prior to the obtaining of the post-administration biological sample or by some other clinical standard; determining that the dose of reference level, wherein BCL2 is modulated when the levels in the post-administration biological sample differ from the measurable threshold or that a patient derives clinical benefit (e.g., tumor shrinkage, improvement in quality of life, improvement in progression free or overall survival).

In some aspects, BCL2 may become transcriptional active or its overexpression is triggered in response to treatment by a chemotherapeutic or a targeted agent involved in blocking pathways involved tumor suppression, genesis, progression, growth, proliferation, migration, cell cycle, cell signaling, DNA damage, genomic instability, metastases, invasion, transformation, differentiation, tolerance, vascular leakage, epithelial mesenchymal transition (EMT), aggregation, angiogenesis, adhesion, development of resistance, addiction to oncogenes and non-oncogenes (cytokines, chemokines, growth factors), alteration of immune surveillance or immune response, alteration of tumor stroma/local environment, endothelial activation, extracellular matrix remodeling, hypoxia and inflammation, immune activation or immune suppression, and survival and/or prevention of cell death by apoptosis, necrosis, or autophagy.

In some aspects, the test compound may be an oligonucleotide compound that comprises an oligomer that hybridizes under physiological conditions to an oligonucleotide sequence selected from SEQ ID NO: 1249 or 1254 or the complements thereof.

In some aspects, the oligomer may comprise an oligomer selected from the group consisting of SEQ ID NOs:1250, 1251, 1252, 1253, 1267-1477 or the complements thereof. In some aspects, the oligomer may comprise an oligomer selected from the group consisting of SEQ ID NOs:1250, 1251, 1289-1358 or the complements thereof. In some aspects, the oligomer comprises SEQ ID NO:1250 or 1251. The oligomer may comprise SEQ ID NO:1251.

In some aspects, the oligomer may be administered in a liposome formulation. In some aspects, the liposome formulation may be an amphoteric liposome formulation. In some aspects, the amphoteric liposome formulation may comprise one or more amphoteric lipids. In some aspects, the amphoteric liposome formulation may be formed from a lipid phase comprising a mixture of lipid components with amphoteric properties.

In some aspects, the mixture of lipid components may be selected from the group consisting of (i) a stable cationic lipid and a chargeable anionic lipid, (ii) a chargeable cationic lipid and chargeable anionic lipid and (iii) a stable anionic lipid and a chargeable cationic lipid.

In some aspects, the lipid components may comprise one or more anionic lipids selected from the group consisting of DOGSucc, POGSucc, DMGSucc, DPGSucc, DGSucc, DMPS, DPPS, DOPS, POPS, DMPG, DPPG, DOPG, POPG, DMPA, DPPA, DOPA, POPA, CHEMS and Cet-P. In some aspects, the lipid components may comprise one or more cationic lipids selected from the group consisting of DMTAP, DPTAP, DOTAP, DC-Chol, MoChol, HisChol, DPIM, CHIM, DORIE, DDAB, DAC-Chol, TC-Chol, DOTMA, DOGS, (C18)2Gly+N,N-dioctadecylamido-glycine, CTAP, CPyC, DODAP and DOEPC.

In some aspets, the lipid phase further may comprise neutral lipids. In some aspects, the neutral lipids may be selected from sterols and derivatives thereof, neutral phospholipids, and combinations thereof. In some aspects, neutral phospholipids may be phosphatidylcholines, sphingomyelins, phosphoethanolamines, or mixtures thereof. The phosphatidylcholines may be selected from the group consisting of POPC, OPPC, natural or hydrogenated soy bean PC, natural or hydrogenated egg PC, DMPC, DPPC or DOPC and derivatives thereof and the phosphatidylethanolamines are selected from the group consisting of DOPE, DMPE, DPPE and derivatives thereof.

In some aspects, the amphoteric liposomes may comprise DOPE, POPC, CHEMS and MoChol. In some aspects, the molar ratio of POPC/DOPE/MoChol/CHEMS may be about 6/24/47/23.

In some aspects, one or more biomarkers may be a protein, and wherein the detection step further comprises assaying the levels of protein in the biological sample using mass spectroscopy or an immunoassay or a combination of the two.

In some aspects, biological sample is blood or blood plasma.

In some aspects, the prior biological sample may be taken from the subject.

Other aspects of the invention may include a method of treating a BCL2 mediated cancer in a subject, comprising: administering the test compound; obtaining one or more additional a biological sample from the subject, subsequent to the administration of said test compound; detecting the levels of one or more of a biomarker in the biological sample, wherein the biomarker is selected from the group consisting of but not limited to: Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof; comparing the levels of said one or more biomarkers to a measurable threshold for each biomarker, wherein each measurable threshold for each biomarker is based on the level of that biomarker present in a prior biological sample obtained prior to the obtaining of the post-administration biological sample or by some other clinical standard; determining that the dose of reference level, wherein BCL2 is modulated when the levels in the post-administration biological sample differ from the measurable threshold or that a patient derives clinical benefit (e.g., tumor shrinkage, improvement in quality of life, improvement in progression free or overall survival).

In some aspects, the method may further comprise collecting a prior biological sample from the subject at the same time or prior to the test compound administration.

In some aspects, the level of leptin may be detected in the sample, and wherein it is determined from the determining step that leptin differs from the statistically determined threshold, initiates a treatment for cancer; and treating the subject with a new or modified treatment for cancer.

In further aspects, the level of Ki-67 may be detected in the sample, and wherein from the determining step that Ki-67 defines patients that are responsive to treatment.

In yet further aspects, the presence of chromosomal rearrangements may be detected in the sample, and wherein from the determining step that chromosomal rearrangements defines patients that are responsive to treatment.

In yet further aspects, the standard uptake value (SUV) may be detected in the PET images, and where in from the determining step that SUV levels defines patients that are responsive to treatment. As used herein, standard uptake values (SUV) determined by FDG-PET refers to the currently accepted method to measure the metabolic uptake of glucose or metabolically active tumors. SUV is often used in PET imaging for a simple semi-quantitative analysiss commonly used in the analysis of [18F]fluorodeoxyglucose ([18F]FDG) images of cancer patients. SUVs are a convenient measure for the evaluation of [18F]FDG PET images within a subject to study identify, monitor therapy, and/or therapy response, and also compare between subjects.

In yet further aspects, the blood sample from the subject may demonstrate lymphocyte or platelet counts and subtype that may be detected in the sample, and where in from the determining step that lymphocyte count defines patients that are responsive to treatment.

In yet further aspects, an immunohistochemistry or flow cytometry panel may be detected in the tumor or blood sample and where in from the determining step that the protein markers defines patients that are response to treatment.

Some aspects of the present invention may comprise a method of modulating the expression of BCL2 in a subject in need thereof, comprising administering a test compound; wherein the modulation of the expression of BCL2 in a subject, modulates the expression of one or more of the following biomarkers but not limited to: Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof. Some embodiments of the present invention may comprise a kit for determining the down-regulation of the expression of BCL2 after administration of a test compound for the treatment of a BCL2 mediated cancer in a subject having cancer comprising: probes for detecting the levels of one or more of a biomarker in the biological sample, wherein the biomarker is selected from the group consisting of but not limited to: Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-10, and IP10 or combinations thereof.

### BRIEF DESCRIPTION OF THE DRAWINGS

FIG. 1 depicts the results of a study where PNT2258 and the chemotherapeutic agents rituximab or docetaxel were administered alone or in combination to immunosuppressed mice bearing human tumors.

FIGS. 2A-D depict patient data and grouping into initial dosing cohort in a dosing and safety trial in human cancer patient subjects and patient data for a proof of concept single arm study. Patient data is also shown, grouped by cancer type.

FIG. 3 depicts the length of time subjects remained in the dose and safety study (measured in days on study), sorted by dosing cohort.

FIGS. 4A-D depict change in BCL-2, active BCL-2, PARP, and caspase-3 expression pre- and post-dose in the dose and safety study subject PBMC cells and change in BCL-2 from pre to post-dose in evaluable single arm proof of concept subject PBMC cells and tumor biopsies.

FIGS. 5A-B depict the relative amount of BCL2 knockdown after administration of PNT-2258 in various cancer cell types of patients in the study.

FIGS. 6A-C depict the number of lymphocytes in the human dose and safety study subjects post-administration of various doses of PNT2258 and the human single arm proof of concept subjects post-administration of 120 mg/m² of PNT2258.

FIGS. 7A-B depict the platelet counts in human dose and safety subjects post-administration of various doses of PNT2258 and the human single arm proof of concept subjects post-administration of 120 mg/m² of PNT2258.

FIG. 8 depicts biomarker expression data from healthy, BALB/c mice treated with PNT2258, scrambled control and an empty liposome control.

FIG. 9 depicts biomarker expression in female C.B-17 SCID mice between 4-6 weeks old. Mice were implanted with WSU-DLCL2 xenograft fragments and treated with PNT2258 or scrambled control when tumors achieved volumes of 300-400 mm³. Red=PNT2258; blue=scrambled control.

FIG. 10 depicts biomarker expression in human patients.

FIG. 11 depicts inflammatory cytokine profiles in patients, comparing pre-dose to post-dose.

FIG. 12 depicts drug interactions between PNT2258, PNT100 and metformin in a Pfeiffer human lymphoma cell line in vitro after 6 days post-administration.

FIG. 13 depicts the change in Ki-67 expression in the human single arm proof of concept subjects from pre to post-administration of PNT2258.

FIG. 14 depicts patient response from the single arm proof of concept study.

FIG. 15 depicts patient diagnoses and molecular characteristics at diagnosis or screening from the single arm proof of concept study.

### DETAILED DESCRIPTION

### I. Definitions

As used herein, “patient” refers to a mammal, including a human.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprins, equines, canines, felines, ayes, etc. and non-vertebrate animals such as Drosophila and C. elegans.

As used herein, an “effective amount” is defined as the amount required to confer a therapeutic effect on the treated patient, and is typically determined based on age, surface area, weight and condition of the patient. The interrelationship of dosages for animals and humans (based on milligrams per meter squared of body surface) is described by Freireich et al., Cancer Chemother. Rep., 50: 219 (1966). Body surface area can be approximately determined from height and weight of the patient. See, e.g., Scientific Tables, Geigy Pharmaceuticals, Ardsley, New York, 537 (1970).

As used herein, the term “wherein said chemotherapy agent is present at less than one half the standard dose” refers to a dosage that is less than one half (e.g., less than 50%, less than 40%, less than 10% or less than 1%) of the minimum value of the standard dosage range used for dosing humans. In some embodiments, the standard dosage range is the dosage range recommended by the manufacturer. In other embodiments, the standard dosage range is the range utilized by a medical doctor in the field. In still other embodiments, the standard dosage range is the range considered the normal standard of care in the field. The particular dosage within the dosage range is determined, for example by the age, weight, and health of the subject as well as the type of cancer being treated.

As used herein, a “response profile” is a subject that is likely to respond to a test compound.

As used herein, “biologically relevant expression range or level” is used to define protein levels, expression, translations in a patient prior to treatment of a test compound.

As used herein, the term “under conditions such that expression of said gene is inhibited” refers to conditions in which an oligonucleotide of the present invention hybridizes to a gene (e.g., a regulatory region of the gene) and inhibits transcription of the gene by at least 10%, at least 25%, at least 50%, or at least 90% relative to the level of transcription in the absence of the oligonucleotide. Exemplary genes include BCL2; additional genes that may be inhibited along with BCL2 include, without limitation, c-ki-ras, c-Ha-ras, c-myc, her-2, and TGF-α.

As used herein, the term “modulation” refers to a regulating according to measure or proportion. Modulation also includes the process of varying one or more properties of gene transcription or protein translation or expression.

As used herein, the term “under conditions such that growth of said cell is reduced” refers to conditions where an oligonucleotide of the present invention, when administered to a cell (e.g., a cancer) reduces the rate of growth of the cell by at least 10%, at least 25%, at least 50% or at least 90% relative to the rate of growth of the cell in the absence of the oligonucleotide.

As used herein, the term Ki-67 is a nuclear protein that is associated with and is a cellular marker for proliferation. Furthermore it is associated with ribosomal RNA transcription. Reducing antigen Ki-67 is indicative of an inhibition gene transcription. Generally, Ki-67 protein is present during all active phases of the cell cycle (G1, S, G2, and mitosis), but is absent from resting cells (G0). Ki-67 is an excellent marker to determine the growth fraction of a given cell population. The fraction of Ki-67-positive tumor cells (the Ki-67 labeling index) is often correlated with the clinical course of cancer.

As used herein, the term immunohistochemistry or flow cytometry panel, refers to immunohistochemical panels to test for leukemia, lymphoma or carcinomas.

As used herein, the term cytogenetics refers to tests to identify gene/chromosomal rearrangements and chromosomal abberations including deletions associated with samples of leukemias, lymphomas, and carcinomas. The primary function of lymphocytes is the formation of antibodies, a common set of genes affected in blood cancer involve the formation of the heavy and light chains of the antibody. These genes are found on the following chromosomes: Heavy chain: chromosome 14, Light chain kappa: chromosome 2, Light chain lambda: chromosome. Besides these genes key genes specific to subsets of leukemia, lymphomas and carcinomas are known. Examples, include, but not meant to be limiting include the (1) c-myc gene located on chromosome 8 and may include t(8;14), t(2;8), t(8;22), (2) bcl-6 located on chromosome 3 and may include: t(3;14), t(2;3) and t(3;22), (3) bcl-3 located on chromosome 19 and may include t(14;19), (4) bcl-1 (Cyclin located on chromosome 11 and may include t(11;14), (5) chromosomal deletions and markers common in chronic lymphocytic leukemia (CLL) and may include 17p, 13p, ZAP70, etc. As used herein, standard uptake values (SUV) determined by FDG-PET refers to the currently accepted method to measure the metabolic uptake of glucose or metabolically active tumors.

As used herein, the term “nucleic acid molecule” refers to any nucleic acid containing molecule, including but not limited to, DNA or RNA. The term encompasses sequences that include any of the known base analogs of DNA and RNA.

The term “gene” refers to a nucleic acid (e.g., DNA) sequence that comprises coding sequences necessary for the production of a polypeptide, precursor or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ or upstream of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the “regulatory region” of a gene is any part of a gene that regulates the expression of a gene, including, without limitation, transcriptional and translational regulation. The regions include without limitation the 5′ and 3′ regions of genes, binding sites for regulatory factors, including without limitation transcription factor binding sites. The regions also include regions that are as long as 20,000 or more base pairs upstream or downstream of translational start sites, so long as the region is involved in any way in the regulation of the expression of the gene. The region may be as short as 20 base pairs or as long as thousands of base pairs.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, micro RNA (miRNA), rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 8 and 100), however, as used herein, the term is also intended to encompass longer polynucleotide chains (e.g., as large as 5000 residues). Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

In some embodiments, oligonucleotides are “antigenes.” As used herein, the term “antigene” refers to an oligonucleotide that hybridizes to the promoter region of a gene. In some embodiments, the hybridization of the antigene to the promoter inhibits expression of the gene.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “completely complementary,” for example when used in reference to an oligonucleotide of the present invention refers to an oligonucleotide where all of the nucleotides are complementary to a target sequence (e.g., a gene).

As used herein, the term “partially complementary,” for example when used in reference to an oligonucleotide of the present invention, refers to an oligonucleotide where at least one nucleotide is not complementary to the target sequence. Exemplary partially complementary oligonucleotides are those that can still hybridize to the target sequence under physiological conditions. The term “partially complementary” refers to oligonucleotides that have regions of one or more non-complementary nucleotides both internal to the oligonucleotide or at either end. Oligonucleotides with mismatches at the ends may still hybridize to the target sequence.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the teem “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions,” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH, PO₄.H₂O and 1.85 g/1 EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/1 NaCl, 6.9 g/1 NaH₂PO₄.H₂O and 1.85 g/1 EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The present invention is not limited to the hybridization of probes of about 500 nucleotides in length. The present invention contemplates the use of probes between approximately 8 nucleotides up to several thousand (e.g., at least 5000) nucleotides in length. One skilled in the relevant understands that stringency conditions may be altered for probes of other sizes (See e.g., Anderson and Young, Quantitative Filter Hybridization, in Nucleic Acid Hybridization[1985] and Sambrook et al., Molecular Cloning—A Laboratory Manual, Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., and supplements through 2006.))

It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “physiological conditions” refers to specific stringency conditions that approximate or are conditions inside an animal (e.g., a human). Exemplary physiological conditions for use in vitro include, but are not limited to, 37° C., 95% air, 5% CO₂, commercial medium for culture of mammalian cells (e.g., DMEM media available from Gibco, Md.), 5-10% serum (e.g., calf serum or horse serum), additional buffers, and optionally hormone (e.g., insulin and epidermal growth factor).

As used herein, the term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “measurable” refers to obtaining and measuring a sample from an animal (e.g., a human) or assessing parameters such as, but not limited to images by CT scan, PET, MRI, X-ray, prognostic score/index (e.g. R-IPI, revised International Prognostic Index) or combinations thereof which may serve to identify or predict outcomes of a disease.

As used herein, the term “purified” or “to purify” refers to the removal of components (e.g., contaminants) from a sample. For example, recombinant polypeptides are expressed in bacterial host cells and the polypeptides are purified by the removal of host cell proteins; the percent of recombinant polypeptides is thereby increased in the sample.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants.” An antigenic determinant may compete with the intact antigen (i.e., the “immunogen” used to elicit the immune response) for binding to an antibody.

As used herein, the term “western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “chemotherapeutic agents” refers to compounds that are useful in the treatment of disease (e.g., cancer). Exemplary chemotherapeutic agents affective against cancer include, but are not limited to, daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES), fluradabine, bendamustine, alkylating agents (e.g., nitrogen mustards, nitrosoureas, tetrazines, aziridines, cisplatins), anti-metabolites (e.g., anti-folates), anti-microtubule agents (e.g., paclitaxel, vinca alkaloids), topoisomerase inhibitors (e.g., irinotecan, topotecan), cytotoxic antibiotics (e.g., doxorubicin, daunorubicin), PARP agents, other targeted agents, such as antibodies, or antibody-like agents. Included within the definition of chemotherapeutic agents are compounds useful in augmenting or the effect of a first chemotherapeutic agent or agents or oligonucleotides of the present invention, or mitigating side effects of a first chemotherapeutic agent or agents or oligonucleotide of the present invention. Examplary targeted agents may also include, for example, inhibitors of kinases, cell surface receptors and proteins/enzymes involved in intracellular and extracellular cell signaling pathways.

Included within the definition of immunotherapy are immunomodulating agents that induce, enhance or suppress the immune response.

Included within the definition of radiotherapy are radiological interventions using X-rays, ultrasound, radiowaves, heat or magnetic fields useful in augmenting the effect of a first chemotherapeutic agent or agents or oligonucleotide of the present invention, or mitigating side effects of a first chemotherapeutic agent or agents or oligonucleotide of the present invention.

Included within the definition of surgical therapy are surgical or invasive interventions (e.g., tumor resection, central catheter placement) useful in augmenting the effect of a first chemotherapeutic agent or agents or oligonucleotide of the present invention, or mitigating side effects of a first chemotherapeutic agent or agents or oligonucleotide of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl or 2-ethylhexyl. An alkyl group can be substituted (i.e., optionally substituted) with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, or hydroxy. Without limitation, some examples of substituted alkyls include carboxyalkyl (such as HOOC-alkyl, alkoxycarbonylalkyl and alkylcarbonyloxyalkyl), cyanoalkyl, hydroxyalkyl, alkoxyalkyl, acylalkyl, hydroxyalkyl, aralkyl, (alkoxyaryl)alkyl, (sulfonylamino)alkyl (such as (alkylsulfonylamino)alkyl), aminoalkyl, amidoalkyl, (cycloaliphatic)alkyl, cyanoalkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl and 2-hexenyl. An alkenyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “amido” encompasses both “aminocarbonyl” and “carbonylamino”. These terms when used alone or in connection with another group refers to an amido group such as N(R^{X})₂—C(O)— or R^{Y}C(O)—N(R^{X})₂— when used terminally and —C(O)—N(R^{X})— or —N(R^{X})—C(O)— when used internally, wherein R^{X} and R^{Y} are defined below. Examples of amido groups include alkylamido (such as alkylcarbonylamino and alkylcarbonylamino), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —NR^{X}R^{Y} wherein each of R^{X} and R^{Y} is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, and arylamino.

When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl). The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl]; sulfonyl [e.g., aliphaticsulfonyl and aminosulfonyl]; sulfinyl [e.g., aliphaticsulfinyl]; sulfanyl [e.g., aliphaticsulfanyl]; nitro; cyano; halo; hydroxyl; mercapto; sulfoxy; urea; thiourea; sulfamoyl; sulfamide; and carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((arylalkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl and ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxyl)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, and bicyclo[3.3.1]nonenyl.

A cycloalkyl or cycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, and (heteroaraliphatic)carbonylamino], nitro, carboxy [e.g., HOOC—, alkoxycarbonyl, and alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl], nitro, cyano, halo, hydroxy, mercapto, sulfonyl [e.g., alkylsulfonyl and arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicyclic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydro-benzofuryl, octahydro-chromenyl, octahydro-thiochromenyl, octahydro-indolyl, octahydro-pyrindinyl, decahydro-quinolinyl, octahydro-benzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicyclic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, and (heteroaraliphatic)carbonylamino], nitro, carboxy [e.g., HOOC-, alkoxycarbonyl, and alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl], nitro, cyano, halo, hydroxy, mercapto, sulfonyl [e.g., alkylsulfonyl and arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring structure having 4 to 15 ring atoms wherein one or more of the ring atoms is a heteroatom (e.g., N, O, S, or combinations thereof) and wherein one ore more rings of the bicyclic or tricyclic ring structure is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thiazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl]; sulfonyl [e.g., aliphaticsulfonyl and aminosulfonyl]; sulfinyl [e.g., aliphaticsulfinyl]; sulfanyl [e.g., aliphaticsulfanyl]; nitro; cyano; halo; hydroxyl; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (amino sulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxyl)heteroaryl; ((carboxy)alkyl)heteroaryl; [((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl, and (haloalkyl)heteroaryl [e.g., trihaloalkylheteroaryl].

A “heteroaraliphatic” (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

As used herein, an “acyl” group refers to a foryl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group or —OC(O)— or —C(O)O—; when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)3- when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —S(O)₂—NR^{X}R^{Y} or —NR^{X}—S(O)₂—R^{Z} when used terminally or —S(O)₂—NR^{X}- or —NR^{X}—S(O)₂— when used internally, wherein R^{X}, R^{Y}, and R^{Z} are defined above.

As used herein a “sulfanyl” group refers to —S—R^{X} when used terminally and —S-when used internally, wherein R^{X} has been defined above. Examples of sulfanyls include alkylsulfanyl.

As used herein a “sulfinyl” group refers to —S(O)—R^{X} when used terminally and —S(O)— when used internally, wherein R^{X} has been defined above.

As used herein, a “sulfonyl” group refers to —S(O)₂—R^{X} when used terminally and —S(O)₂— when used internally, wherein R^{X} has been defined above.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidino” group refers to the structure —N═C(N(R^{X}R^{Y}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

The terms “terminally” and “internally” refer to the location of a group within a substituent. A group is terminal when the group is present at the end of the substituent not further bonded to the rest of the chemical structure. Carboxyalkyl, i.e., R^{X}O (O)C-alkyl is an example of a carboxy group used terminally A group is internal when the group is present in the middle of a substituent to at the end of the substituent bound to the to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables contained herein encompass specific groups, such as alkyl and aryl. Unless otherwise noted, each of the specific groups for the variables contained herein can be optionally substituted with one or more substituents described herein. Each substituent of a specific group is further optionally substituted with one to three of halo, cyano, oxoalkoxy, hydroxyl, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxyl, amino, nitro, aryl, haloalkyl, and alkyl. As an additional example, the cycloalkyl portion of a (cycloalkyl)carbonylamino can be optionally substituted with one to three of halo, cyano, alkoxy, hydroxyl, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, co-therapies include any oligonucleotide compounds that can be used alone or in combination with other cancer therapies to treat cancer.

As used herein, factors constituting Revised International Prognostic Index (R-IPI) include: age >60, performance status >2, elevated lactate dehydrogenase, >1 extranodal site, stage 3/4 disease.

### II. Cancers

Compounds and methods of the present invention may be used to treat several types of cancer. Examples of cancers that can be treated in some embodiments with compounds and methods of the present invention include solid tumor cancers, including, but not limited to melanoma, metastatic melanoma, non-small cell lung cancer (NSCLC), small cell lung cancer (SCLC), multiple myeloma, chronic lymphocytic leukemia (CLL), small lymphocytic lymphoma (SLL), acute myeloid leukemia (AML), metastatic hormone refractory prostate cancer, breast cancer, ovarian cancer, thyroid cancer, pancreatic cancer, head and neck cancer, and hematological cancers including, but not limited to, all leukemias and lymphomas.

Compounds and methods of the present invention may be used to treat several types of lymphoma subtypes selected from Hodgkin lymphoma, classical Hodgkin lymphoma, lymphocyte-rich/mixed cellularity/lymphocyte depleted, lymphocyte-rich, mixed cellularity, lymphocyte-depleted, nodular sclerosis, classical Hodgkin lymphoma NOS, nodular lymphocyte predominant Hodgkin lymphoma, non-Hodgkin lymphoma, non-Hodgkin lymphoma B-cell, precursor non-Hodgkin lymphoma B-cell, mature non-Hodgkin lymphoma B-cell, chronic/small/prolymphocytic/mantle B-cell NHL, chronic/small lymphocytic leuk/lymph, prolymphocytic leukemia B-cell, mantle-cell lymphoma, lymphoplasmacytic lymphoma/Waldenstrom, lymphoplasmacytic lymphoma, waldenstrom macroglubulinemia, diffuse large B-cell lymphoma (DLBCL), DLBCL NOS, intravascular large B-cell lymphoma, primary effusion lymphoma, mediastinal large B-cell lymphoma, Burkitt lymphoma/leukemia, marginal-zone lymphoma (MZL), splenic MZL, extranodal MZL MALT type, nodal MZL, follicular lymphoma, hairy-cell leukemia, plasma cell neoplasms, plasmacytoma, multiple myeloma/plasma-cell leuk, heavy chain disease, non-Hodgkin lymphoma B-cell NOS, non-Hodgkin lymphoma T-cell, precursor non-Hodgkin lymphoma T-cell, mature Non-Hodgkin lymphoma T-cell, mycosis fungoides/Sezary syndrome, mycosis fungoides, Sezary syndrome, peripheral T-cell lymphoma, peripheral T-cell lymphom NOS, angioimmunoblastic T-cell lymphoma, subcutaneous panniculitis-like T-cell lymphoma, anaplastic large cell lymphoma T- or Null-cell, hepatosplenic T-cell lymphoma, enteropathy-type T-cell lymphoma, cutaneous T-cell lymphoma NOS, primary cutaneous anaplastic large cell lymphoma, adult T-cell leukemia/lymphoma, NK/T-cell lymph., nasal-type/aggressive NK leuk, T-cell large granular lymphocytic leukemia, prolymphocytic leukemia T-cell, non-Hodgkin lymphoma NOS T-cell, non-Hodgkin lymphoma—unknown lineage, precursor lymphoblastic leuk/lymph—unknown lineage, prolymphocytic leukemia—unknown lineage, non-Hodgkin lymphoma NOS—unknown lineage, composite Hodgkin lymphoma and NHL, lymphoid neoplasm NOS, and unclassified subtypes.

Melanoma, or cancer of the skin, is a very common form of cancer, and if diagnosed and treated early can generally be managed. However, if untreated, melanoma can lead to metastatic melanoma and is difficult to treat. Development of stage III or IV melanoma is a serious medical condition and can lead to death usually in 8 to 18 months from the time of diagnosis.

Dacarbazine is the only chemotherapeutic agent approved by the FDA to treat metastatic melanoma, and is associated with a response rate of 7-12% and a median survival of 5.6-7.8 months after the initiation of treatment. Combinations with other chemotherapeutic agents have not shown improvement in response rate. Recently, other agents including ipilimumab, a monoclonal antibody that blocks cytotoxic T-lymphocyte associated antigen 4 (CTLA-4) in combination with dacarbazine, have been shown to have better survival rates than dacarbazine alone. More recently, vemurafenib (PLX4032), a potent inhibitor of mutated BRAF kinase inhibitor showed improved survival in metastatic melanoma patients with the BRAF V600E mutation when compared to dacarbazine.

Approximately 40-60% of cutaneous melanoma carry mutations in the BRAF kinase inhibitor, which leads to the constitutive activation of downstream signaling through the MAPK pathway. Although most (approximately 90%) of the mutations consist of glutamic acid for valine at codon 600 (BRAF V600E), other activating mutations are known, such as BRAF V600K, and BRAF V600R. Targeting the BRAF V600E mutation has lead the discovery and development of vemurafenib and to an improved overall and progression-free survival in patients selected for the BRAF V600E mutation.

However, patients without the BRAF V600E mutation would appear to have no other treatment alternative other than dacarbazine, the only chemotherapeutic agent approved by the FDA to treat metastatic melanoma. For either treatment choice, the overall survival for any metastatic melanoma patients is generally less than two years.

In other embodiments, the compositions or oligomers of the present invention can be used for treating inflammation disorders such as rheumatoid arthritis, lupis, and inflammatory bowel disease, with or without additional therapeutic agents including TNF-alpha inhibitors such as etanercept, nonsteriodal anti-inflammatory drugs (NSAIDs) such as ibuprofen, corticosteroids, disease modifying antirheumatic drugs (DMARDs) such as methotrexate, and immuno suppressants such as azathioprine, and a CD-20 inhibitor.

### III. Cancer Therapies

Cancer therapies of the present invention include oligonucleotide compounds, chemotherapy agents, radiation therapy, surgery, or combinations thereof.

A. Gene Targets of Oligonucleotide Compounds

1. BCL2

In many types of human tumors, including lymphomas and leukemias, the human BCL2 gene is overexpressed, and may be associated with tumorigenicity (Tsujimoto et al., Science 228:1440-1443 [1985]). BCL2 has been found in many forms of both hematologic and solid tumors. These include all solid tumor cancers, including, but not limited to melanoma, metastatic melanoma, non-small cell lung cancer (NSCLC), small cell lung cancer (SCLC), acute myeloid leukemia (AML), metastatic hormone refractory prostate cancer, breast cancer, ovarian cancer, thyroid cancer, pancreatic cancer, head and neck cancer, and hematological cancers including, but not limited to, all leukemias and lymphomas.

High levels of expression of the human BCL2 gene have been found in all lymphomas with t (14; 18) chromosomal translocations including most follicular B cell lymphomas and many large cell non-Hodgkin's lymphomas. High levels of expression of the BCL2 gene have also been found in certain leukemias that do not have a t(14; 18) chromosomal translation, including most cases of chronic lymphocytic leukemia acute, many lymphocytic leukemias of the pre-B cell type, neuroblastomas, nasopharyngeal carcinomas, and many adenocarcinomas of the prostate, breast and colon. (Reed et al., Cancer Res. 51:6529 [1991]; Yunis et al., New England J. Med. 320:1047; Campos et al., Blood 81:3091-3096 [1993]; McDonnell et al., Cancer Res. 52:6940-6944 [1992); Lu et al., Int. J Cancer 53:29-35 [1993]; Bonner et al., Lab Invest. 68:43A [1993]).

The current model proposes that BCL2 proteins work in a hierarchical network of inhibitory interactions to regulate apoptosis. BCL2 family proteins are essential regulators of apoptosis that contribute to the deregulation of survival pathways in cancer cells. Pro-survival members of the family, such as BCL2, BCL-XL and MCL-1, possess four BCL2 homology (BH) domains. Pro-apoptotic BCL2 proteins are divided into two sub-families. Proteins such as BAX or BAK contain BH1-BH3 domains but lack the N-terminal BH4 domain. Proteins such as BAD, BID, BIM or PUMA lack all but the BH3 domain and are known as the ‘BH3-only’ proteins. In healthy cells, the pro-apoptotic effects of BAX and BAK are restrained by the pro-survival proteins BCL2, BCL-XL and MCL-1.

However, in response to pro-apoptotic stresses, members of the BH3-only proteins are expressed or activated. BH3-only proteins inhibit the pro-survival effects of BCL2, BCL-XL and MCL-1 thereby liberating the pro-apoptotic effects of BAX and BAK leading to cell death.

The deregulation of apoptosis is a defining characteristic of malignant cells and it is a process in which the overexpression of the BCL2 protein plays a key role. The elevated BCL2/anti-apoptotic phenotype contributes to the chemo-resistance of a broad variety of tumors including diffuse large B-cell lymphoma and many solid tumors. Given this biological importance, BCL2 is a prime target for drug discovery. Previous approaches to modulating BCL2 have included RNA-targeted antisense oligonucleotides, small molecule protein inhibitors and others

2. Other Oncogene Targets

The present invention may include the co-administration of oligonucleotides designed for other oncogene targets, such as c-erb-2 (her-2), c-myc, TGF-α, c-Ha-ras, and c-ki-Ras. Other exemplary oncogenes include, but are not limited to, BCR/ABL, ABL1/BCR, ABL, BCL1, BCL-2, BRAF, CD24, CDK4, EGFR/ERBB-1, HSTF1, INT1/WNT1, INT2, MDM2, MET, MYB, MYC, MYCN, MYCL1, RAF1, NRAS, REL, AKT2, APC, BCL2-ALPHA, BCL2-BETA, BCL3, BCR, BRCA1, BRCA2, CBL, CCND1, CDKN1A, CDKN1C, CDKN2A, CDKN2B, CRK, CRK-II, CSF1R/FMS, DBL, DDOST, DCC, DPC4/SMAD4, E-CAD, E2F1/RBAP, ELK1, ELK3, EPH, EPHA1, E2F1, EPHA3, ERG, ETS1, ETS2, FER, FGR, FLI1/ERGB2, FOS, FPS/FES, FRA1, FRA2, FYN, HCK, HEK, HER3/ERBB-2, ERBB-3, HER4/ERBB-4, HST2, INK4A, INK4B, JUN, JUNB, JUND, KIP2, KIT, KRAS2A, KRAS2B, LCK, LYN, MAS, MAX, MCC, MLH1, MOS, MSH2, MYBA, MYBB, NF1, NF2, P53, PDGFB, PIM1, PTC, RB1, RET, ROS1, SKI, SRC1, TAL1, TGFBR2, THRA1, THRB, TIAM1, TRK, VAV, VHL, WAF1, WNT2, WT1, YES1, ALK/NPM1, AMI1, AXL, FMS, GIP, GLI, GSP, HOX11, HST, IL3, INT2, KS3, K-SAM, LBC, LMO-1, LMO-2, L-MYC, LYL1, LYT-10, MDM-2, MLH1, MLL, MLM, N-MYC, OST, PAX-5, PMS-1, PMS-2, PRAD-1, RAF, RHOM-1, RHOM-2, SIS, TAL2, TAN1, TIAM1, TSC2, TRK, TSC1, STK11, PTCH, MEN1, MEN2, P57/KIP2, PTEN, HPC1, ATM, XPA/XPG, BCL6, DEK, AKAP13, CDH1, BLM, EWSR1/FLI1, FES, FGF3, FGF4, FGF6, FANCA, FLI1/ERGB2, FOSL1, FOSL2, GL1, HRAS1, HRX/MLLT1, HRX/MLLT2, KRAS2, MADH4, MAS1, MCF2, MLLT1/MLL, MLLT2/HRX, MTG8/RUNX1, MYCLK1, MYH11/CBFB, NFKB2, NOTCH1, NPM1/ALK, NRG/REL, NTRK1, PBX1/TCF3, PML/RARA, PRCA1, RUNX1, RUNX1/CBFA2T1, SET, TCF3/PBX1, TGFB1, TLX1, P53, WNT1, WNT2, WT1, αv-β3, PKCα, TNFα, Clusterin, Survivin, TGFβ, c-fos, c-SRC, and INT-1.

3. Non-Oncogene Targets

The present invention is not limited to co-administration of oligonucleotides effective against other oncogenes. For example, in some embodiments, the genes to be targeted include, but are not limited to, an immunoglobulin or antibody gene, a clotting factor gene, a protease, a pituitary hormone, a protease inhibitor, a growth factor, a somatomedian, a gonadotrophin, a chemotactin, a chemokine, a plasma protein, a plasma protease inhibitor, an interleukin, an interferon, a cytokine, a transcription factor, or a pathogen target (e.g., a viral gene, a bacterial gene, a microbial gene, a fungal gene).

Examples of specific genes include, but are not limited to, ADAMTS4, ADAMTS5, APOA1, APOE, APP, B2M, COX2, CRP, DDX25, DMC1, FKBP8, GH1, GHR, IAPP, IFNA1, IFNG, IL1, Il10, IL12, IL13, IL2, IL4, IL7, IL8, IPW, MAPK14, Meil, MMP13, MYD88, NDN, PACE4, PRNP, PSEN1, PSEN2, RAD51, RAD51C, SAP, SNRPN, TLR4, TLR9, TTR, UBE3A, VLA-4, and PTP-1B, c-RAF, m-TOR, LDL, VLDL, ApoB-100, VEGF, rhPDGF-BB, NADs, ICAM-1, MUC1, 2-dG, CTL, PSGL-1, E2F, NF-kB, HIF, and GCPRs.

In other embodiments a gene from a pathogen is targeted. Exemplary pathogens include, but are not limited to, Human Immunodeficiency virus, Hepatitis B virus, hepatitis C virus, hepatitis A virus, respiratory syncytial virus, pathogens involved in severe acute respiratory syndrome, West Nile virus and foodborne pathogens (e.g., E. coli).

B. Oligonucleotide Design

In some embodiments, the present invention provides antigene oligonucleotides for modulating the expression of oncogenes, such as BCL2. Exemplary design and production strategies for antigenes are described below. The description below is not intended to limit the scope of antigene compounds suitable for use in the present invention and that other antigenes are within the scope of the present invention.

a. Regulatory Regions of the Oncogenes

The BCL2 gene has two promoters designated P1 and P2. P1 from which most BCL2 mRNA is transcribed is located approximately 1.4 kb upstream of the translation initiation site and P2 is 1.3 kb downstream of P1. (See Seto, M. et al. EMBO J. 7, 123-131 (1988).) P1 is GC-rich, lacks a TATA box, has many transcription start sites and includes seven consensus binding sites for the SP1 transcription factor. P2 includes a CCAAT box and a TATA box and has two different transcription initiation sites. There are multiple NF-κB recognition sites and an SV40 enhancer-like octamer motif within P2. (See Heckman, C. A., et al. Oncogene 21, 3898-3908 (2002).) (See SEQ ID NO:1254.) Most human follicular lymphomas contain t(14;18) chromosomal translocations that result from 3′-BCL2 gene region breakpoints. (See Tsujimoto, Y. et al. Proc. Natl. Acad. Sci. U. S. A 84, 1329-1331 (1987).) These translocations place BCL2 expression under control of the immunoglobulin heavy chain (IgH) locus enhancer resulting in upregulation of BCL2 expression. Alternatively, there are 5′-BCL2 breakpoint regions that result from fusions with either the IgH locus or two different immunoglobulin light chain (IgL) loci that are found in some DLCL lymphoma patient isolates. (See Yonetani, N. et al. Jpn. J. Cancer Res. 92, 933-940 (2004) These 5′-BCL2 breakpoints have been mapped in separate heterogeneous patient isolates to a region spanning 378 to 2312 bp upstream of the translation initiation site. (See SEQ ID NOs:1255-1266.) The importance of regulatory regions surrounding bcl-2 have been recognized by others. For example, researchers have demonstrated that a series of 20 base deletions between the P1 and P2 promoter of BCL-2 decreased transcription (Young and Korsmeyer Mol. Cell Biol 13: p 3686-3697 (1993) and Chen H M, Boxer L M. Mol Cell Biol. 15: p. 3840-3847 (11995)); Miyashita et. al. reported that p53 dependent regions upstream of the BCL-2 gene act as negative regulatory elements (Cancer Res. 54: p. 3131-3135(1994)); and Duan et. al. showed long range regulatory effects on BCL-2 transcription by enhancers in the IgH 3′ region (Oncogene 27: p. 6720-6728 (2008)). Regions around the breakpoints may be sequences that can be used for BCL2 oligonucleotide design.

b. Oligonucleotide Design

The oligonucleotides can include any oligomer that hybridizes to the upstream regions of the BCL2 gene, defined as SEQ ID NOs:1249 and 1254.

In some embodiments, oligonucleotides are designed based on preferred design criteria. Such oligonucleotides can then be tested for efficacy using the methods disclosed herein. For example, in some embodiments, the oligonucleotides are methylated on at least one, two or all of the CpG islands. In other embodiments, the oligonucleotides contain no methylation. The present invention is not limited to a particular mechanism. Indeed, an understanding of the mechanism is not necessary to practice the present invention. Nonetheless, it is contemplated that oligonucleotides in some embodiments are those that have at least a 50% GC content and at least two GC dinucleotides. Also, in some embodiments, the oligonucleotides do not self hybridize. In further embodiments, oligonucleotides are designed with at least 1 A or T to minimize self hybridization. In yet further embodiments, commercially available computer programs are used to survey oligonucleotides for the ability to self hybridize. In still other embodiments, oligonucleotides are at least 10, or 15 nucleotides and no more than 100 nucleotides in length. In further embodiments, oligonucleotides are 18-26 nucleotides in length. In additional embodiments, oligonucleotides comprise the universal protein binding sequences CGCCC and CGCG or the complements thereof.

In some embodiments, oligonucleotides hybridize to a promoter region of a gene upstream from the TATA box of the promoter. In further embodiments, oligonucleotides are designed to hybridize to regions of the promoter region of an oncogene known to be bound by proteins (e.g., transcription factors). In some embodiments, oligonucleotide compounds are not completely homologous to other regions of the human genome. The homology of the oligonucleotide compounds of the present invention to other regions of the genome can be determined using available search tools (e.g., BLAST, available at the Internet site of NCBI).

The present invention is not limited to the oligonucleotides described herein. Other suitable oligonucleotides may be identified (e.g., using the criteria described above or other criteria). Candidate oligonucleotides may be tested for efficacy using any suitable method. For example, candidate oligonucleotides can be evaluated for their ability to prevent cell proliferation at a variety of concentrations. In some embodiments, oligonucleotides inhibit gene expression or cell proliferation at a low concentration (e.g., less than 20 μM, or 10 μM in in vitro assays.).

c. Oligonucleotide Zones

In some embodiments, regions within the promoter region of an oncogene are further defined as regions for hybridization of oligonucleotides. In some embodiments, these regions are referred to as “hot zones.”

In some embodiments, hot zones are defined based on oligonucleotide compounds that are demonstrated to be effective (see above section on oligonucleotides) and those that are contemplated to be effective based on the criteria for oligonucleotides described above. In some embodiments, hot zones encompass 10 bp upstream and downstream of each compound included in each hot zone and have at least one CG or more within an increment of 40 bp further upstream or downstream of each compound. In further embodiments, hot zones encompass a maximum of 100 bp upstream and downstream of each oligonucleotide compound included in the hot zone. In additional embodiments, hot zones are defined at beginning regions of each promoter. These hot zones are defined either based on effective sequence(s) or contemplated sequences and have a preferred maximum length of 200 bp. Based on the above described criteria, exemplary hot zones were designed. The hot zones for BCL2 are located at bases 679-720, 930-1050, 1070-1280, and 1420-1760 of SEQ ID NO:1249.

d. Description

In one aspect, the oligonucleotides can be any oligomer that hybridizes under physiological conditions to the following sequences: SEQ ID NO:1249 or SEQ ID NO:1254. In another aspect, the oligomer can be any oligomer that hybridizes to nucleotides 500-2026, nucleotides 500-1525, nucleotides 800-1225, nucleotides 900-1125, nucleotides 950-1075 or nucleotides 970-1045 of SEQ ID NO:1249 or the complement thereof. In another aspect, the oligonucleotides can be any oligomer that hybridizes under physiological conditions to exemplary hot zones in SEQ ID NO:1249. Examples of oligomers include, without limitation, those oligomers listed in SEQ ID NOS:1250-1253 and 1267-1477 and the complements thereof. In another aspect, the oligonucleotides are SEQ ID NOs 2-22, 283-301, 463-503, 937-958, 1082-1109, 1250-1254 and 1270-1477 and the complements thereof. In an embodiment of these aspects, the oligonucleotides are from 15-35 base pairs in length.

In one embodiment, the oligomer can be SEQ ID NO:1250, 1251, 1252, 1253, 1267-1477 or the complement thereof. In another embodiment, the oligomer can be SEQ ID NO: 1250, 1251, 1267, 1268, 1276, 1277, 1285, 1286 or the complement thereof. In yet another embodiment, the oligomer can be SEQ ID NOs 1250, 1251, 1289-1358 or the complements thereof. In still another embodiment the oligomer can be SEQ ID NO:1250 or 1251.

In a further embodiment of these aspects, the oligomer has the sequence of the positive strand of the BCL2 sequence, and thus, binds to the negative strand of the sequence.

In other aspects, the oligomers can include mixtures of anti-BCL2 oligonucleotides. For instance, the oligomer can include multiple oligonucleotides each of which hybridizes to different parts of SEQ ID NOs:1249 and 1254. Oligomers can hybridize to overlapping regions on those sequences or the oligomers may hybridize to non-overlapping regions. In other embodiments, oligomers can be SEQ ID NOs:1250, 1251, 1252, 1253, 1267-1477 or the complement thereof, wherein the mixture of anti-BCL2 oligomers comprises oligomers of at least 2 different sequences.

In other embodiments, the oligomer can include a mixture of oligomers, each of which hybridizes to a regulatory region of different genes. For instance, the oligomer can include a first oligomer that hybridizes to SEQ ID NO:1249 or 1254 and second oligomer that hybridizes to a regulatory region of a second gene. In some embodiments, the oligomer includes an oligomer of SEQ ID NOs 1250-1254 and 1267-1477 or the complements thereof, In other embodiments, the oligomer includes SEQ ID NO 1250 or 1251 or the complement thereof and an oligomer that hybridizes to the promoter region of another oncogene, such as c-erb-2 (her-2), c-myc, TGF-α, c-Ha-ras, and c-ki-Ras. Examples of such oligomers may be found in, for example, U.S. Pat. Nos. 7,524,827; 7,807,647; and 7,498,315.

In some embodiments, the present invention provides oligonucleotide therapeutics that are methylated at specific sites. The present invention is not limited to a particular mechanism. Indeed, an understanding of the mechanism is not necessary to practice the present invention. Nonetheless, it is contemplated that one mechanism for the regulation of gene activity is methylation of cytosine residues in DNA. 5-methylcytosine (5-MeC) is the only naturally occurring modified base detected in DNA (Ehrlick et al., Science 212:1350-1357 (1981)). Although not all genes are regulated by methylation, hypomethylation at specific sites or in specific regions in a number of genes is correlated with active transcription (Doerfler, Annu. Rev. Biochem. 52:93-124 [1984]; Christman, Curr. Top. Microbiol. Immunol. 108:49-78 [1988]; Cedar, Cell 34:5503-5513 [1988]). DNA methylation in vitro can prevent efficient transcription of genes in a cell-free system or transient expression of transfected genes. Methylation of C residues in some specific cis-regulatory regions can also block or enhance binding of transcriptional factors or repressors (Doerfler, supra; Christman, supra; Cedar, Cell 34:5503-5513 (1988); Tate et al., Curr. Opin. Genet. Dev. 3:225-231 [1993]; Christman et al., Virus Strategies, eds. Doerfler, W. & Bohm, P. (VCH, Weinheim, N.Y.) pp. 319-333 [1993]).

Disruption of normal patterns of DNA methylation has been linked to the development of cancer (Christman et al., Proc. Natl. Acad. Sci. USA 92:7347-7351 [1995]). The 5-MeC content of DNA from tumors and tumor derived cell lines is generally lower than normal tissues (Jones et al., Adv. Cancer Res 40:1-30 [1983]). Hypomethylation of specific oncogenes such as c-myc, c-Ki-ras and c-Ha-ras has been detected in a variety of human and animal tumors (Nambu et al., Jpn. J. Cancer (Gann) 78:696-704 [1987]; Feinberg et al., Biochem. Biophys. Res. Commun 111:47-54 [1983]; Cheah et al., JNCI73:1057-1063 [1984]; Bhave et al., Carcinogenesis (Lond) 9:343-348 [1988]. In one of the best studied examples of human tumor progression, it has been shown that hypomethylation of DNA is an early event in development of colon cancer (Goetz et al., Science 228:187-290 [1985]). Interference with methylation in vivo can lead to tumor formation. Feeding of methylation inhibitors such as L-methionine or 5-azacytodine or severe deficiency of 5-adenosine methionine through feeding of a diet depleted of lipotropes has been reported to induce formation of liver tumors in rats (Wainfan et al., Cancer Res. 52:2071s-2077s [1992]). Studies show that extreme lipotrope deficient diets can cause loss of methyl groups at specific sites in genes such as c-myc, ras and c-fos (Dizik et al., Carcinogenesis 12:1307-1312 [1991]). Hypomethylation occurs despite the presence of elevated levels of DNA MTase activity (Wainfan et al., Cancer Res. 49:4094-4097 [1989]). Genes required for sustained active proliferation become inactive as methylated during differentiation and tissue specific genes become hypomethylated and are active. Hypomethylation can then shift the balance between the two states. In some embodiments, the present invention thus takes advantage of this naturally occurring phenomena, to provide compositions and methods for site specific methylation of specific gene promoters, thereby preventing transcription and hence translation of certain genes. In other embodiments, the present invention provides methods and compositions for upregulating the expression of a gene of interest (e.g., a tumor suppressor gene) by altering the gene's methylation patterns.

An understanding that mammalian cell promoter regions are surrounded by CpG islands and that these non-methylated regions contribute to gene regulation is emerging (Blackledge N P, Klose R J (2011) Epigenetics 6: p. 147-152 and Deaton A M, Bird A (2011) Genes Dev. 25: p. 1010-1022). These genomic regions surrounding promoters are DNAse I-hypersensitive have also enabled the discovery of cis-regulatory elements that act as transcription factors, enhancers, silencers, repressors, or control regions, which regulate gene expression (Thurman R E, Rynes E, Humbert R, Vierstra H, Maurano M T (2012) Nature 489: 75-82; Maston et al. Annu. Rev. Genomics Hum. Genet. 2006. 7:29-59; Sabo P J, Kuehn M S, Thurman R, Johnson, B E, Johnson, B E et al (2006) Nat Methods 3: p. 511-8). Additionally, higher-order secondary structures (quadruplexes, cruciforms or I-motifs), which surround the promoter regions of oncogenes, may also serve as cis-regulatory domains to modulate transcription (Brazda V, Laister R C, Jagelska E B, Arrowsmith, C (2011) BMC Mol Biol 12: p. 33-48 and Kendrick, S. and L. H. Hurley, Pure Appl Chem, 2010. 82(8): p. 1609-1621. In other embodiments, the present invention provides methods and compositions that can hybridize or bind the hypomethylated or unmethylated CG-rich areas (CpG islands).

The present invention is not limited to the use of methylated oligonucleotides. Indeed, the use of non-methylated oligonucleotides for the modulation of gene expression is specifically contemplated by the present invention. Experiments conducted during the course of development of the present invention demonstrated that an unmethylated oligonucleotide targeted toward BCL2 inhibited the growth of lymphoma cells to a level that was comparable to that of a methylated oligonucleotide.

Both SEQ ID NOs:1250 and 1251 are included within the scope of the term PNT-100 as used below. PNT 100 is a 24-base DNA oligonucleotide sequence designed to target a region found within the t(14,18) translocation known to drive certain lymphomas. Subsequent examples use the unmethylated form, but the term PNT-100 is inclusive of the methylated form.

C. Preparation and Formulation of Oligonucleotides

Any of the known methods of oligonucleotide synthesis can be used to prepare the modified oligonucleotides of the present invention. In some embodiments utilizing methylated oligonucleotides the nucleotide, dC is replaced by 5-methyl-dC where appropriate, as taught by the present invention. The modified or unmodified oligonucleotides of the present invention are most conveniently prepared by using any of the commercially available automated nucleic acid synthesizers. They can also be obtained from commercial sources that synthesize custom oligonucleotides pursuant to customer specifications.

While oligonucleotides are one form of compound, the present invention comprehends other oligomeric oligonucleotide compounds, including but not limited to oligonucleotide mimetics such as are described below. The oligonucleotide compounds in accordance with this invention typically comprise from about 18 to about 30 nucleobases (i.e., from about 18 to about 30 linked bases), although both longer and shorter sequences may find use with the present invention.

Specific examples of compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their intemucleoside backbone can also be considered to be oligonucleosides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In some embodiments the oligonucleotides have a phosphorothioate backbone having the following general structure.

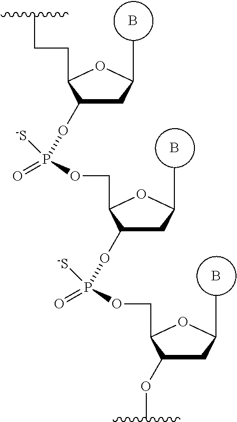

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts.

In other oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991) and Neilsen, Methods in Enzymology, 313, 156-164 (1999). PNA compounds can be obtained commercially, for example, from Applied Biosystems (Foster City, Calif., USA).

In some embodiments, oligonucleotides of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂, and —O—N(CH₃)—CH₂—CH₂— [wherein the native phosphodiester backbone is represented as —O—P—O—CH₂-] of the above referenced U.S. Pat. No. 5,489,677, and the amide backbones of the above referenced U.S. Pat. No. 5,602,240. Also exemplary are oligonucleotides having morpholino backbone structures of the above-referenced U.S. Pat. No. 5,034,506.

Oligonucleotides can also have sugars other than ribose and deoxyribose, including arabinofuranose (described in International Publication number WO 99/67378, which is herein incorporated by reference), xyloarabinofuranose (described in U.S. Pat. Nos. 6,316,612 and 6,489,465, which are herein incorporated by reference), α-threofuranose (Schoning, et al. (2000) Science, 290, 1347-51, which is herein incorporated by reference) and L-ribofuranose. Sugar mimetics can replace the sugar in the nucleotides. They include cyclohexene (Wang et al.(2000) J. Am. Chem. Soc. 122, 8595-8602; Vebeure et al. Nucl. Acids Res. (2001) 29, 4941-4947, which are herein incorporated by reference), a tricyclo group (Steffens, et al. J. Am. Chem. Soc. (1997) 119, 11548-11549, which is herein incorporated by reference), a cyclobutyl group, a hexitol group (Maurinsh, et al. (1997) J. Org. Chem, 62, 2861-71; J. Am. Chem. Soc. (1998) 120, 5381-94, which are herein incorporated by reference), an altritol group (Allart, et al., Tetrahedron (1999) 6527-46, which is herein incorporated by reference), a pyrrolidine group (Scharer, et al., J. Am. Chem. Soc., 117, 6623-24, which is herein incorporated by reference), carbocyclic groups obtained by replacing the oxygen of the furnaose ring with a methylene group (Froehler and Ricca, J. Am. Chem. Soc. 114, 8230-32, which is herein incorporated by reference) or with an S to obtain 4′-thiofuranose (Hancock, et al., Nucl. Acids Res. 21, 3485-91, which is herein incorporated by reference), and/or morpholino group (Heasman, (2002) Dev. Biol., 243, 209-214, which is herein incorporated by reference) in place of the pentofuranosyl sugar. Morpholino oligonucleotides are commercially available from Gene Tools, LLC (Corvallis Oreg., USA).

The oligonucleotides can also include “locked nucleic acids” or LNAs. The LNAs can be bicyclic, tricyclic or polycyclic. LNAs include a number of different monomers, one of which is depicted in Formula I.

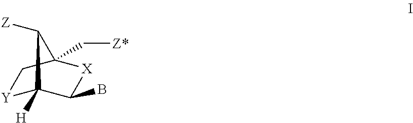

wherein
B constitutes a nucleobase;
Z* is selected from an internucleoside linkage and a terminal group;
Z is selected from a bond to the intemucleoside linkage of a preceding nucleotide/nucleoside and a terminal group, provided that only one of Z and Z* can be a terminal group;
X and Y are independently selected from —O—, —S—, —N(H)—, —N(R)—, —CH₂— or —C(H)=, CH₂—O—, —CH₂—S—, —CH₂—N(H)—, —CH₂—N(R)—, —CH₂—CH₂— or —CH₂—C(H)═, —CH═CH—; provided that X and Y are not both O.

In addition to the LNA [2′-Y,4′-C-methylene-β-D-ribofuranosyl]monomers depicted in formula I (a [2,2,1]bicyclo nucleoside), an LNA nucleotide can also include “locked nucleic acids” with other furanose or other 5 or 6-membered rings and/or with a different monomer formulation, including 2′-Y,3′ linked and 3′-Y,4′ linked, 1′-Y,3 linked, 1′-Y,4′ linked, 3′-Y,5′ linked, 2′-Y, 5′linked, 1′-Y,2′ linked bicyclonucleosides and others. All the above mentioned LNAs can be obtained with different chiral centers, resulting, for example, in LNA [3′-Y-4′-C-methylene (or ethylene)-β (or α)-arabino-, xylo- or L-ribo-furanosyl]monomers. LNA oligonucleotides and LNA nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, Colo. 80301 USA.

Oligonucleotides can also contain one or more substituted sugar moieties. Oligonucleotides can comprise one of the following at the 2′ sugar position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Yet other oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide or a group improving pharmacodynamic properties of an oligonucleotide and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further modification includes 2′-dimethylaminooxyethoxy (i.e., an O(CH₂)₂ON(CH₃), group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, isocytosine, pseudoisocytosine, 5-bromouracil, 5-propynyluracil, 5-propynylcytosine, 5-propyny-6-fluoroluracil, 5-methylthiazoleuracil, 6-aminopurine, 2-aminopurine, inosine, diaminopurine, 7-deazaguanine, 7-deazaadenine, 3-deazaguanine, 3-deazaadenine, 8-azaguanine, 8-azaadenine, 7-propyne-7-deazaadenine, 7-propyne-7-deazaguanine, 2-chloro-6-aminopurine, 4-acetylcytosine, 5-hydroxymethylcytosine, 8-hydroxy-N6-methyladenosine, aziridinylcytosine, 5-(carboxyhydroxyl-methyl) uracil, 5-fluorouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, N6-methyladenine, 7-methylguanine and other alkyl derivatives of adenine and guanine, 2-propyl adenine and other alkyl derivatives of adenine and guanine, 2-aminoadenine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 2-thiothymine, 5-halouracil, 5-halocytosine, 6-azo uracil, cytosine and thymine, 5-methyl-2-thiouracil, 2-thiouracil, 4-thiouracil, 5-methyluracil, 8-halo, 8-amino, 8-thiol, 8-hydroxyl and other 8-substituted adenines and guanines, 5-trifluoromethyl uracil and cytosine, N-uracil-5-oxyacetic acid methylester, uracil-5-oxyacetic acid, queosine, xanthine, hypoxanthine, 2-thiocytosine and 2,6-diaminopurine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by −0.6-1.2° C. These are particularly effective when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes pharmaceutical compositions and formulations that include the oligomeric compounds of the present invention as described below.

D. Oligonucleotide Cocktails

In some embodiments, the present invention provides cocktails comprising two or more oligonucleotides directed toward regulatory regions of genes (e.g., oncogenes). In some embodiments, two or more oligonucleotides hybridize to different regions of a regulatory region of the same gene. In other embodiments, the two or more oligonucleotides hybridize to regulatory regions of two different genes. The present invention is not limited to a particular mechanism. Indeed, an understanding of the mechanism is not necessary to practice the present invention. Nonetheless, it is contemplated that the combination of two or more compounds of the present invention provides an inhibition of cancer cell growth that is greater than the additive inhibition of each of the compounds administered separately.

E. Index of SEQ IDs

| | |
|---|---|
| SEQ ID NO: 1249 | BCL2 upstream region |
| SEQ ID NO: 1250 | PNT-100 oligonucleotide methylated |
| SEQ ID NO: 1251 | PNT-100 oligonucleotide not methylated |
| SEQ ID NO: 1252 | anti-BCL2 oligonucleotide methylated |
| SEQ ID NO: 1253 | anti-BCL2 oligonucleotide not methylated |
| SEQ ID NO: 1254 | BCL2 secondary promoter sequence |
| SEQ ID NOs: 1255-1266 | BCL2 sequences |
| SEQ ID NOs: 1250-1254 | anti-BCL2 oligonucleotides |
| and 1267-1477 | |
| and 1289-1358 | |
| SEQ ID NOs: 1448-1461 | BCL2 control oligonucleotides |

G. Other Cancer Therapies

The present invention may be used to test the effectiveness of test compounds as chemotherapy agents to down-regulate BCL2 levels in subjects having BCL2 mediated cancers.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds include both known and potential therapeutic compounds. A test compound can be determined to be therapeutic by screening using the screening methods of the present invention. In some embodiments of the present invention, test compounds include antisense compounds.

a. Chemotherapy Agents

Chemotherapy agents of the present invention can include any suitable chemotherapy drug or combinations of chemotherapy drugs (e.g., a cocktail). Exemplary chemotherapy agents include, without limitation, alkylating agents, platinums, anti-metabolites, anthracyclines, taxanes, camptothecins, nitrosoureas, EGFR inhibitors, antibiotics, HER2/neu inhibitors, BRAF inhibitors, NRAS or RAS inhibitors, angiogenesis inhibitors, kinase inhibitors, proteaosome inhibitors, immunotherapies, hormone therapies, photodynamic therapies, cancer vaccines, histone deacetylase inhibitors, sphingolipid modulators, oligomers, other unclassified chemotherapy drugs and combinations thereof.

Chemotherapy agents can include cocktails of two or more chemotherapy drugs mentioned above. In several embodiments, a chemotherapy agent is a cocktail that includes two or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, oligomers or combinations thereof.

1. Alkylating Agents

Alkylating agents are chemotherapy agents that are thought to attack the negatively charged sites on the DNA (e.g., the oxygen, nitrogen, phosphorous and sulfur atoms) and bind to the DNA thus altering replication, transcription and even base pairing. It is also believed that alkylation of the DNA also leads to DNA strand breaks and DNA strand cross-linking. By altering DNA in this manner, cellular activity is effectively stopped and the cancer cell will die. Common alkylating agents include, without limitation, procarbazine, ifosphamide, cyclophosphamide, bendamustine, melphalan, chlorambucil, dacarbazine, busulfan, thiotepa, and the like. Dacarbazine for Injection is indicated in the treatment of metastatic malignant melanoma. In addition, injections of dacarbazine are also indicated for Hodgkin's disease as a second-line therapy when used in combination with other effective agents. Alkylating agents such as those mentioned above can be used in combination with one or more other alkylating agents and/or with one or more chemotherapy agents of a different class(es).

2. Platinums

Platinum chemotherapy agents are believed to inhibit DNA synthesis, transcription and function by cross-linking DNA subunits. (The cross-linking can happen either between two strands or within one strand of DNA.) Common platinum chemotherapy agents include, without limitation, cisplatin, carboplatin, oxaliplatin, Eloxatin™, and the like. Platinum chemotherapy agents such as those mentioned above can be used in combination with one or more other platinums and/or with one or more chemotherapy agents of a different class(es).

3. Anti-Metabolites

Anti-metabolite chemotherapy agents are believed to interfere with normal metabolic pathways, including those necessary for making new DNA. Common anti-metabolites include, without limitation, Methotrexate, 5-fluorouracil (e.g., capecitabine), gemcitabine (2′-deoxy-2′,2′-difluorocytidine monohydrochloride ((3-isomer), Eli Lilly), 6-mercaptopurine, 6-thioguanine, fludarabine, cladribine, cytarabine, tegafur, raltitrexed, cytosine arabinoside, and the like. Gallium nitrate is another anti-metabolite that inhibits ribonucleotides reductase. Anti-metabolites such as those mentioned above can be used in combination with one or more other anti-metabolites and/or with one or more chemotherapy agents of a different class(es).

4. Anthracyclines

Anthracyclines are believed to promote the formation of free oxygen radicals. These radicals result in DNA strand breaks and subsequent inhibition of DNA synthesis and function. Anthracyclines are also thought to inhibit the enzyme topoisomerase by forming a complex with the enzyme and DNA. Common anthracyclines include, without limitation, daunorubicin, doxorubicin, idarubicin, epirubicin, mitoxantrone, adriamycin, bleomycin, mitomycin-C, dactinomycin, mithramycin and the like. Anthracyclines such as those mentioned above can be used in combination with one or more other anthracyclines and/or with one or more chemotherapy agents of a different class(es).

5. Taxanes

Taxanes are believed to bind with high affinity to the microtubules during the M phase of the cell cycle and inhibit their nomial function. Common taxanes include, without limitation, paclitaxel, docetaxel (Taxotere™), Taxol™, taxasm, 7-epipaclitaxel, t-acetyl paclitaxel, 10-desacetyl-paclitaxel, 10-desacetyl-7-epipaclitaxel, 7-xylosylpaclitaxel, 10-desacetyl-7-epipaclitaxel, 7-N-N-dimethylglycylpaclitaxel, 7-L-alanylpaclitaxel and the like. Taxanes such as those mentioned above can be used in combination with one or more other taxanes and/or with one or more chemotherapy agents of a different class(es).

For instance, Taxotere™ is indicated for the treatment of patients with locally advanced or metastatic breast cancer after failure of prior chemotherapy; in combination with doxorubicin and cyclophosphamide is indicated for the adjuvant treatment of patients with operable node-positive breast cancer; as a single agent, is indicated for the treatment of patients with locally advanced or metastatic non-small cell lung cancer (NSCLC) after failure of prior platinum-based chemotherapy; in combination with cisplatin is indicated for the treatment of patients with unresectable, locally advanced or metastatic NSCLC who have not previously received chemotherapy for this condition; in combination with prednisone is indicated for the treatment of patients with androgen-independent (hormone-refractory) metastatic prostate cancer; in combination with cisplatin and fluorouracil is indicated for the treatment of patients with advanced gastric adenocarcinoma, including adenocarcinoma of the gastroesophageal junction, who have not received prior chemotherapy for advanced disease; and in combination with cisplatin and fluorouracil is indicated for the induction treatment of patients with locally advanced squamous cell carcinoma of the head and neck (SCCHN).

6. Camptothecins

Camptothecins are thought to complex with topoisomerase and DNA resulting in the inhibition and function of this enzyme. It is further believed that the presence of topoisomerase is required for on-going DNA synthesis. Common camptothecins include, without limitation, irinotecan, topotecan, etoposide, vinca alkaloids (e.g., vincristine, vinblastine or vinorelbine), amsacrine, teniposide and the like. Camptothecins such as those mentioned above can be used in combination with one or more other camptothecins and/or with one or more chemotherapy agents of a different class(es).

7. Nitrosoureas

Nitrosoureas are believed to inhibit changes necessary for DNA repair. Common nitrosoureas include, without limitation, carmustine (BCNU), lomustine (CCNU), semustine and the like. Nitrosoureas such as those mentioned above can be used in combination with one or more other nitrosoureas and/or with one or more chemotherapy agents of a different class(es).

8. EGFR Inhibitors

EGFR (i.e., epidermal growth factor receptor) inhibitors are thought to inhibit EGFR and interfere with cellular responses including cell proliferation and differentiation. EGFR inhibitors include molecules that inhibit the function or production of one or more EGFRs. They include small molecule inhibitors of EGFRs, antibodies to EGFRs, antisense oligomers, RNAi inhibitors and other oligomers that reduce the expression of EGFRs. Common EGFR inhibitors include, without limitation, gefitinib, erlotinib (Tarceva®), cetuximab (Erbitux™), panitumumab (Vectibix®, Amgen) lapatinib (GlaxoSmithKline), CI1033 or PD183805 or canternib (6-acrylamide-N-(3-chloro-4-flurorphenyl)-7-(3-morpholinopropoxy)quinazolin-4-amine, Pfizer), and the like. Other inhibitors include PKI-166 (4-[(1R)-1-phenylethylamino]-6-(4-hydroxyphenyl)-7H-pyrrolo[2,3-d]pyrimidine, Novartis), CL-387785 (N-[4-(3-bromoanilino)quinazolin-6-yl]but-2-ynamide), EKB-569 (4-(3-chloro-4-fluroranilino)-3-cyano-6-(4-dimethylaminobut2(E)-enamido)-7-ethoxyquinoline, Wyeth), lapatinib (GW2016, GlaxoSmithKline), EKB509 (Wyeth), panitumumab (ABX-EGF, Abgenix), matuzumab (EMD 72000, Merck), and the monoclonal antibody RH3 (New York Medical). EGFR inhibitors such as those mentioned above can be used in combination with one or more other EGFR inhibitors and/or with one or more chemotherapy agents of a different class(es).

9. Antibiotics

Antibiotics are thought to promote the formation of free oxygen radicals that result in DNA breaks leading to cancer cell death. Common antibiotics include, without limitation, bleomycin and rapamycin and the like. The macrolide fungicide rapamycin (also called RAP, rapamune and sirolimus) binds intracellularly to the to the immunophilin FK506 binding protein 12 (FKBP12) and the resultant complex inhibits the serine protein kinase activity of mammalian target of rapamycin (mTOR). Rapamycin macrolides include naturally occurring forms of rapamycin as well as rapamycin analogs and derivatives that target and inhibit mTOR. Other rapamycin macrolides include, without limitation, temsirolimus (CCI-779, Wyeth), everolimus and ABT-578. Antibiotics such as those mentioned above can be used in combination with one or more other antibiotics and/or with one or more chemotherapy agents of a different class(es).

10. HER2/neu Inhibitors

HER2/neu Inhibitors are believed to block the HER2 receptor and prevent the cascade of reactions necessary for tumor survival. Her2 inhibitors include molecules that inhibit the function or production of Her2. They include small molecule inhibitors of Her2, antibodies to Her2, antisense oligomers, RNAi inhibitors and other oligomers that reduce the expression of tyrosine kinases. Common HER2/neu inhibitors include, without limitation, trastuzumab (Herceptin®, Genentech) and the like. Other Her2/neu inhibitors include bispecific antibodies MDX-210(FCγR1-Her2/neu) and MDX-447 (Medarex), pertuzumab (rhuMAb 2C4, Genentech), HER2/neu inhibitors such as those mentioned above can be used in combination with one or more other HER2/neu inhibitors and/or with one or more chemotherapy agents of a different class(es).

11. Angiogenesis Inhibitors

Angiogenesis inhibitors are believed to inhibit vascular endothelial growth factor, i.e., VEGF, thereby inhibiting the founation of new blood vessels necessary for tumor life. VEGF inhibitors include molecules that inhibit the function or production of one or more VEGFs. They include small molecule inhibitors of VEGF, antibodies to VEGF, antisense oligomers, RNAi inhibitors and other oligomers that reduce the expression of tyrosine kinases. Common angiogenesis inhibitors include, without limitation, bevacizumab (Avastin®, Genentech). Other angiogenesis inhibitors include, without limitation, ZD6474 (AstraZeneca), BAY-43-9006, sorafenib (Nexavar®, Bayer), semaxanib (SU5416, Pharmacia), SU6668 (Pharmacia), ZD4190 (N-(4-bromo-2-fluorophenyl)-6-methoxy-7-[2-(1H-1,2,3-triazol-1-yl)ethoxy]quinazolin-4-amine, Astra Zeneca), Zactima™ (ZD6474, N-(4-bromo-2-fluorophenyl)-6-methoxy-7-[2-(1H-1,2,3-triazol-1-yl)ethoxy]quinazolin-4-amine, Astra Zeneca), vatalanib, (PTK787, Novartis), the monoclonal antibody IMC-1C11 (Imclone) and the like. Angiogenesis inhibitors such as those mentioned above can be used in combination with one or more other angiogenesis inhibitors and/or with one or more chemotherapy agents of a different class(es).

12. BRAF Inhibitors

The B-Raf (BRAF) variant, BRAF V600E, is the most frequent oncogenic protein kinase mutation known. The selection of potent and selective inhibitory agents to active BRAF V600E has led to a number of agents that show BRAF kinase specificity and cytotoxic effects to cells bearing the BRAF V600E mutation. In particular, the Plexxikon agent, PLX4720, was reported as demonstrating specific ERK phosphorylation in BRAF V600E but not BRAF wild-type tumor cells. In melanoma models, PLX4720 induced cell cycle arrest and apoptosis in B-Raf V600E positive cells. The Plexxikon agent, vemurafenib (PLX4032), another B-Raf V600E specific agent, was tested in humans with metastatic melanoma with the BRAF V600E. A significant treatment effect was observed for improved overall survival and progression free survival.

As noted above, although most (approximately 90%) of the mutations consist of glutamic acid for valine at codon 600 (BRAF V600E), other activating mutations are known, such as BRAF V600K, and BRAF V600R.

BRAF V600E and “wild-type” BRAF has been associated many cancers, including for example, Non-Hodgkin's lymphoma, leukemia, malignant melanoma, thyroid, colorectal, and adenocarcinoma and NSCLC.

Other BRAF inhibitors that may be used in embodiments of the present invention include, but are not limited to, GDC-0879, BAY 7304506 (regorafenib), RAF265 (CHIR-265), SB590885, Sorafenib.

13. Other Kinase Inhibitors

In addition to EGFR, HER2, BRAF and VEGF inhibitors, other kinase inhibitors are used as chemotherapeutic agents. Aurora kinase inhibitors include, without limitation, compounds such as 4-(4-N benzoylamino)aniline)-6-methyxy-7-(3-(1-morpholino)propoxy)quinazoline (ZM447439, Ditchfield et al., J. Cell. Biol., 161:267-80 (2003)) and hesperadin (Haaf et al., J. Cell Biol., 161: 281-94 (2003)). Other compounds suitable for use as Aurora kinase inhibitors are described in Vankayalapati H, et al., Mol. Cancer Ther. 2:283-9 (2003). SRC/Abl kinase inhibitors include without limitation, AZD0530 (4-(6-chloro-2,3-methylenedioxyanilino)-7-[2-(4-methylpiperazin-1-yl)ethoxy]-5-tetrahycropyran-4-yloxyquinazoline). Tyrosine kinase inhibitors include molecules that inhibit the function or production of one or more tyrosine kinases. They include small molecule inhibitors of tyrosine kinases, antibodies to tyrosine kinases and antisense oligomers, RNAi inhibitors and other oligomers that reduce the expression of tyrosine kinases. CEP-701 and CEP-751 (Cephalon) act as tyrosine kinase inhibitors. Imatinib mesylate is a tyrosine kinase inhibitor that inhibits bcr-abl by binding to the ATP binding site of bcr-abl and competitively inhibiting the enzyme activity of the protein. Although imatinib is quite selective for bcr-abl, it does also inhibit other targets such as c-kit and PDGF-R. FLT-3 inhibitors include, without limitation, tandutinib (MLN518, Millenium), sutent (SU11248, 5-[5-fluoro-2-oxo-1,2-dihydroindol-(3Z)-ylidenemethyl]-2,4-dimethyl-1H-pyrrole-3-carboxylic acid [2-diethylaminoethyl]amide, Pfizer), midostaurin (4′-N-benzoyl staurosporine, Novartis), lefunomide (SU101) and the like. MEK inhibitors include, without limitation, 2-(2-Chloro-4-iodo-phenylamino)-N-cyclopropylmethoxy-3,4-difluoro-benzamide (PD184352/CI-1044, Pfizer), PD198306 (Pfizer), PD98059 (2′-amino-3′-methoxyflavone), UO126 (Promega), Ro092-210 from fermented microbial extracts (Roche), the resorcyclic acid lactone, L783277, also isolated from microbial extracts (Merck) and the like. Tyrosine kinase inhibitors such as those mentioned above can be used in combination with one or more other tyrosine kinase inhibitors and/or with one or more chemotherapy agents of a different class(es) including phosphatidylinositide 3-kinase inhibitors, Bruton's tyrosine kinase inhibitors and spleen tyrosine kinase (also known as Syk protein (encoded by the SYK gene)) inhibitors without limitation.

14. Proteosome Inhibitors

Proteaosome inhibitors are believed to inhibit the breakdown of some of these proteins that have been marked for destruction. This results in growth arrest or death of the cell. Common proteosome inhibitors include, without limitation, bortezomib, ortezomib, carfilzomib and the like. Proteosome inhibitors such as those mentioned above can be used in combination with one or more other proteaosome inhibitors and/or with one or more chemotherapy agents of a different class(es).

15. Immunotherapies

Immunotherapies are thought to bind to and block specific targets, thereby disrupting the chain of events needed for tumor cell proliferation. Common immunotherapies include, without limitation, rituximab and other antibodies directed against CD19, CD20, CD38, Campath-1H™ and other antibodies directed against CD-50, epratuzmab and other antibodies directed against CD-22, galiximab and other antibodies directed atainst CD-80, apolizumab HU1D10 and other antibodies directed against HLA-DR, and the like. Radioisotopes can be conjugated to the antibody, resulting in radioimmunotherapy. Two such anti-CD20 products are tositumomab (Bexxar™) and ibritumomab (Zevalin™). Immunotherapies such as those mentioned above can be used in combination with one or more other immunotherapies and/or with one or more chemotherapy agents of a different class(es). Antibodies or compositions that bind or block CD38, CD19 and CD20 and antibodies that stimulate T-cell mediated killing such as PD-1.

Rituximab (Rituxan™), among other indications, is indicated for the treatment of patients with previously untreated follicular, CD20-positive, B-cell non-Hodgkin's lymphoma; and previously untreated and previously treated CD20-positive chronic lymphocytic leukemia in combination with fludarabine and cyclophosphamide (FC).

Yervoy™ (ipilimumab) is a monoclonal antibody that blocks a molecule known as cytotoxic T-lymphocyte antigen or CTLA-4. CTLA-4 may play a role in slowing down or turning off the body's immune system, affecting its ability to fight off cancerous cells. Yervoy may work by allowing the body's immune system to recognize, target, and attack cells in melanoma tumors. The drug is administered intravenously. Yervoy is indicated for the treatment of unresectable or metastatic melanoma. Yervoy (3 mg/kg) is administered intravenously over 90 minutes every 3 weeks for a total of four doses. Two key clinical trials have been conducted with Yervoy. The first which resulted in FDA approval based on Yervoy's safety and effectiveness in a single international study of 676 patients with melanoma. All patients in the study had stopped responding to other FDA-approved or commonly used treatments for melanoma. In addition, participants had disease that had spread or that could not be surgically removed.

Other CTLA-4 antibodies, which may be used in embodiments of the present invention include, but are not limited to tremelimumab.

16. Hormone Therapies

Hormone therapies are thought to block cellular receptors, inhibit the in vivo production of hormones, and/or eliminate or modify hormone receptors on cells, all with the end result of slowing or stopping tumor proliferation. Common hormone therapies include, without limitation, antiestrogens (e.g., tamoxifen, toremifene, fulvestrant, raloxifene, droloxifene, idoxifene and the like), progestogens e.g., megestrol acetate and the like) aromatase inhibitors (e.g., anastrozole, letrozole, exemestane, vorozole, exemestane, fadrozole, aminoglutethimide, exemestane, 1-methyl-1,4-androstadiene-3,17-dione and the like), anti-androgens (e.g., bicalutimide, nilutamide, flutamide, cyproterone acetate, and the like), luteinizing hormone releasing hormone agonist (LHRH Agonist) (e.g., goserelin, leuprolide, buserelin and the like); 5-α-reductase inhibitors such as finasteride, and the like.

Abiraterone (Zytiga™) is another useful hormone therapy, which inhibits the enzyme 17 α-hydroxylase/C17,20 lyase in testicular, prostate, and adrenal cancer tissue, blocking the synthesis of precursors of testosterone. Hormone therapies such as those mentioned above can be used in combination with one or more other hormone therapies and/or with one or more chemotherapy agents of a different class(es).

17. Photodynamic Therapies

Photodynamic therapies expose a photosensitizing drug to specific wavelengths of light to kill cancer cells. Common photodynamic therapies include, for example, porfimer sodium (e.g., Photofrin®) and the like. Photodynamic therapies such as those mentioned above can be used in combination with one or more other photodynamic therapies and/or with one or more chemotherapy agents of a different class(es).

18. Cancer Vaccines

Cancer vaccines are thought to utilize whole, inactivated tumor cells, whole proteins, peptide fragments, viral vectors and the like to generate an immune response that targets cancer cells. Common cancer vaccines include, without limitation, modified tumor cells, peptide vaccine, dendritic vaccines, viral vector vaccines, heat shock protein vaccines and the like.

19. Histone Deacetylase Inhibitors

Histone deacetylase inhibitors are able to modulate transcriptional activity and consequently, can block angiogenesis and cell cycling, and promote apoptosis and differentiation. Histone deacetylase inhibitors include, without limitation, SAHA (suberoylanilide hydroxamic acid), depsipeptide (FK288) and analogs, Pivanex™ (Titan), CI994 (Pfizer), MS275 PXD101 (CuraGen, TopoTarget) MGCD0103 (MethylGene), LBH589, NVP-LAQ824 (Novartis) and the like and have been used as chemotherapy agents. Histone deacetylase inhibitors such as those mentioned above can be used in combination with one or more other histone deacetylase inhibitors and/or with one or more chemotherapy agents of a different class(es).

20. Sphingolipid Modulators

Modulators of Sphingolipid metabolism have been shown to induce apoptosis. For reviews see N. S. Radin, Biochem J, 371:243-56 (2003); D. E. Modrak, et al., Mol. Cancer Ther, 5:200-208 (2006), K. Desai, et al., Biochim Biophys Acta, 1585:188-92 (2002) and C. P. Reynolds, et al. and Cancer Lett, 206, 169-80 (2004), all of which are incorporated herein by reference. Modulators and inhibitors of various enzymes involved in sphingolipid metabolism can be used as chemotherapeutic agents.

(a) Ceramide has been shown to induce apoptosis, consequently, exogenous ceramide or a short-chain ceramide analog such as N-acetylsphingosine (C₂-Cer), C₆-Cer or C₈-Cer has been used. Other analogs include, without limitation, Cer 1-glucuronide, poly(ethylene glycol)-derivatized ceramides and pegylated ceramides.

(b) Modulators that stimulate ceramide synthesis have been used to increase ceramide levels. Compounds that stimulate serine palmitoyltransferase, an enzyme involved in ceramide synthesis, include, without limitation, tetrahydrocannabinol (THC) and synthetic analogs and anandamide, a naturally occurring mammalian cannabinoid. Gemcitabine, retinoic acid and a derivative, fenretinide [N-(4-hydroxyphenyl)retinamide, (4-HPR)], camptothecin, homocamptothecin, etoposide, paclitaxel, daunorubicin and fludarabine have also been shown to increase ceramide levels. In addition, valspodar (PSC833, Novartis), a non-immunosuppressive non-ephrotoxic analog of cyclosporin and an inhibitor of p-glycoprotein, increases ceramide levels.

(c) Modulators of sphingomyelinases can increase ceramide levels. They include compounds that lower GSH levels, as GSH inhibits sphingomyelinases. For example, betathine (β-alanyl cysteamine disulfide), oxidizes GSH, and has produced good effects in patients with myeloma, melanoma and breast cancer. COX-2 inhibitors, such as celecoxib, ketoconazole, an antifungal agent, doxorubicin, mitoxantrone, D609 (tricyclodecan-9-yl-xanthogenate), dexamethasone, and Ara-C(1-β-D-arabinofuranosylcytosine) also stimulate sphingomyelinases.

(d) Molecules that stimulate the hydrolysis of glucosylceramide also raise ceramide levels. The enzyme, GlcCer glucosidase, which is available for use in Gaucher's disease, particularly with retinol or pentanol as glucose acceptors and/or an activator of the enzyme can be used as therapeutic agents. Saposin C and analogs thereof, as well as analogs of the anti-psychotic drug, chloropromazine, may also be useful.

(e) Inhibitors of glucosylceramide synthesis include, without limitation, PDMP (N-[2-hydroxy-1-(4-morpholinylmethyl)-2-phenylethyldecanamide]), PMPP (D,L-threo-(1-phenyl-2-hexadecanoylamino-3-morpholino-1-propanol), P4 or PPPP (D-threo-1-phenyl-2-palmitoylamino-3-pyrrolidino-1-propanol), ethylenedioxy-P4, 2-decanoylamine-3-morpholinoprophenone, tamixofen, raloxifene, mifepristone (RU486), N-butyl deoxynojirimycin and anti-androgen chemotherapy (bicalutamide+leuprolide acetate)). Zavesca®, (1,5-(butylimino)-1,5-dideoxy-D-glucitol) usually used to treat Gaucher's disease, is another inhibitor of glucosylceramide synthesis.

(f) Inhibitors of ceramidase include, without limitation, N-oleoylethanolamine, a truncated form of ceramide, D-MAPP (D-erythro-2-tetradecanoylamino-1-phenyl-1-propanol) and the related inhibitor B13 (p-nitro-D-MAPP).

(g) Inhibitors of sphingosine kinase also result in increased levels of ceramide. Inhibitors include, without limitation, safingol (L-threo-dihydrosphingosine), N,N-dimethyl sphingosine, trimethyl sphingosine and analogs and derivatives of sphingosine such as dihydrosphingosine, and myriocin.

(h) Fumonisins and fumonisin analogs, although they inhibit ceramide synthase, also increase levels of sphinganine due to the inhibition of de novo sphingolipid biosynthesis, resulting in apoptosis.

(i) Other molecules that increase ceramide levels include, without limitation, miltefosine (hexadecylphosphocholine). Sphingolipid modulators, such as those mentioned above, can be used in combination with one or more other sphingolipid modulators and/or with one or more chemotherapy agents of a different class(es).

21. Other Oligomers

In addition to the oligonucleotides presented above, other oligonucleotides have been used as cancer therapies. They include Genasense® (oblimersen, G3139, from Genta), an antisense oligonucleotide that targets BCL2 and G4460 (LR3001, from Genta) another antisense oligonucleotide that targets cancer pathways including, but not limited to STAT-3, survivin, c-myb, and others. Other oligomers include, without limitation, siRNAs, decoys, RNAi oligonucleotides and the like. Oligonucleotides, such as those mentioned above, can be used in combination with one or more other oligonucleotide inhibitors and/or with one or more chemotherapy agents of a different class(es).

22. Other Chemotherapy Drugs

Additional unclassified chemotherapy agents are described in Table 1 below.

TABLE 1
Additional unclassified chemotherapy agents.

| Generic Name | Brand Name | Manufacturer |
|---|---|---|
| aldesleukin | Proleukin ™ | Chiron Corp., |
| (des-alanyl-1, serine-125 human interleukin-2) | | Emeryville, CA |
| alemtuzumab | Campath ™ | Millennium and |
| (IgG1κ anti CD52 antibody) | | ILEX Partners, LP, |
| | | Cambridge, MA |
| alitretinoin | Panretin ™ | Ligand |
| (9-cis-retinoic acid) | | Pharmaceuticals, |
| | | Inc., San Diego CA |
| allopurinol | Zyloprim ™ | GlaxoSmithKline, |
| (l,5-dihydro-4 H-pyrazolo[3,4-d]pyrimidin-4- | | Research Triangle |
| one monosodium salt) | | Park, NC |
| altretamine | Hexalen ™ | US Bioscience, |
| (N,N,N′,N′,N″,N″,-hexamethyl-1,3,5-triazine- | | West |
| 2,4,6-triamine) | | Conshohocken, PA |
| amifostine | Ethyol ™ | US Bioscience |
| (ethanethiol,2-[(3-aminopropyl)amino]-, | | |
| dihydrogen phosphate (ester)) | | |
| anastrozole | Arimidex ™ | AstraZeneca |
| (1,3-Benzenediacetonitrile,a,a,a′,a′- | | Pharmaceuticals, |
| tetramethyl-5-(1H-1,2,4-triazol-1-ylmethyl)) | | LP, Wilmington, |
| | | DE |
| arsenic trioxide | Trisenox ™ | Cell Therapeutic, |
| | | Inc., Seattle, WA |
| asparaginase | Elspar ™ | Merck & Co., Inc., |
| (L-asparagine amidohydrolase, type EC-2) | | Whitehouse Station, NJ |
| BCG Live | TICE BCG ™ | Organon Teknika, |
| (lyophilized preparation of an attenuated strain | | Corp., Durham, NC |
| of Mycobacterium bovis (Bacillus Calmette- | | |
| Gukin [BCG], substrain Montreal) | | |
| bexarotene capsules | Targretin ™ | Ligand |
| (4-[1-(5,6,7,8-tetrahydro-3,5,5,8,8- | | Pharmaceuticals |
| pentamethyl-2-napthalenyl) ethenyl] benzoic | | |
| acid) | | |
| bexarotene gel | Targretin ™ | Ligand |
| | | Pharmaceuticals |
| carmustine with polifeprosan 20 implant | Gliadel Wafer ™ | Guilford |
| | | Pharmaceuticals, |
| | | Inc., Baltimore, MD |
| celecoxib | Celebrex ™ | Searle |
| (as 4-[5-(4-methylphenyl)-3-(trifluoromethyl)- | | Pharmaceuticals, |
| 1H-pyrazol-1-yl] benzenesulfonamide) | | England |
| chlorambucil | Leukeran ™ | GlaxoSmithKline |
| (4-[bis(2chlorethyl)amino]benzenebutanoic acid) | | |
| cladribine | Leustatin, 2- | R. W. Johnson |
| (2-chloro-2′-deoxy-b-D-adenosine) | CdA ™ | Pharmaceutical |
| | | Research Institute, |
| | | Raritan, NJ |
| dacarbazine | DTIC-Dome ™ | Bayer AG, |
| (5-(3,3-dimethyl-1-triazeno)-imidazole-4- | | Leverkusen, |
| carboxamide (DTIC)) | | Germany |
| dactinomycin, actinomycin D | Cosmegen ™ | Merck |
| (actinomycin produced by Streptomyces | | |
| parvullus, C₆₂H₈₆N₁₂O₁₆) | | |
| darbepoetin alfa | Aranesp ™ | Amgen, Inc., |
| (recombinant peptide) | | Thousand Oaks, CA |
| denileukin diftitox | Ontak ™ | Seragen, Inc., |
| (recombinant peptide) | | Hopkinton, MA |
| dexrazoxane | Zinecard ™ | Pharmacia & |
| ((S)-4,4′-(1-methyl-1,2-ethanediyl)bis-2,6- | | Upjohn Company |
| piperazinedione) | | |
| dromostanolone propionate | Dromostanolone ™ | Eli Lilly & |
| (17b-Hydroxy-2a-methyl-5a-androstan-3-one | | Company, |
| propionate) | | Indianapolis, IN |
| dromostanolone propionate | Masterone | Syntex, Corp., Palo |
| | injection ™ | Alto, CA |
| Elliott′s B Solution | Elliott's B | Orphan Medical, |
| | Solution ™ | Inc |
| epoetin alfa | Epogen ™ | Amgen, Inc |
| (recombinant peptide) | | |
| estramustine | Emcyt ™ | Pharmacia & |
| (estra-1,3,5(10)-triene-3,17-diol(17(beta))-, 3- | | Upjohn Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| exemestane | Aromasin ™ | Pharmacia & |
| (6-methylenandrosta-1,4-diene-3,17-dione) | | Upjohn Company |
| filgrastim | Neupogen ™ | Amgen, Inc |
| (r-metHuG-CSF) | | |
| floxuridine (intraarterial) | FUDR ™ | Roche |
| (2′-deoxy-5-fluorouridine) | | |
| fulvestrant | Faslodex ™ | IPR |
| (7-alpha-[9-(4,4,5,5,5-penta | | Pharmaceuticals, |
| fluoropentylsulphinyl) nonyl]estra-1,3,5-(10)- | | Guayama, Puerto |
| triene-3,17-beta-diol) | | Rico |
| gemtuzumab ozogamicin | Mylotarg ™ | Wyeth Ayerst |
| (anti-CD33 hP67.6) | | |
| hydroxyurea | Hydrea ™ | Bristol-Myers |
| | | Squibb |
| ifosfamide | IFEX ™ | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| imatinib mesilate | Gleevec ™ | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| interferon alpha-2a | Roferon-A ™ | Hoffmann-La |
| (recombinant peptide) | | Roche, Inc., Nutley, |
| | | NJ |
| interferon alpha-2b | Intron A ™ | Schering AG, |
| (recombinant peptide) | (Lyophilized | Berlin, Germany |
| | Betaseron) | |
| irinotecan HCl | Camptosar ™ | Pharmacia & |
| ((4S)-4,11-diethyl-4-hydroxy-9-[(4-piperi- | | Upjohn Company |
| dinopiperidino)carbonyloxy]-1H-pyrano[3′,4′: | | |
| 6,7] indolizino[1,2-b] quinoline-3,14(4H,12H) | | |
| dione hydrochloride trihydrate) | | |
| letrozole | Femara ™ | Novartis |
| (4,4′-(1H-1,2,4-Triazol-1-ylmethylene) | | |
| dibenzonitrile) | | |
| leucovorin | Wellcovorin ™, | Immunex, Corp., |
| (L-Glutamic acid, N[4[[(2-amino-5-formyl- | Leucovorin ™ | Seattle, WA |
| 1,4,5,6,7,8-hexahydro-4oxo-6- | | |
| pteridinyl)methyl]amino]benzoyl], calcium salt (1:1)) | | |
| levamisole HCl | Ergamisol ™ | Janssen Research |
| ((—)-(S)-2,3,5,6-tetrahydro-6-phenylimidazo | | Foundation, |
| [2,1-b] thiazole monohydrochloride | | Titusville, NJ |
| C₁₁H₁₂N₂S•HCl) | | |
| lomustine | CeeNU ™ | Bristol-Myers |
| (1-(2-chloro-ethyl)-3-cyclohexyl-1-nitrosourea) | | Squibb |
| meclorethamine, nitrogen mustard | Mustargen ™ | Merck |
| (2-chloro-N-(2-chloroethyl)-N- | | |
| methylethanamine hydrochloride) | | |
| megestrol acetate | Megace ™ | Bristol-Myers |
| 17α(acetyloxy)-6-methylpregna-4,6-diene- | | Squibb |
| 3,20-dione | | |
| melphalan, L-PAM | Alkeran ™ | GlaxoSmithKline |
| (4-[bis(2-chloroethyl) amino]-L-phenylalanine) | | |
| mercaptopurine, 6-MP | Purinethol ™ | GlaxoSmithKline |
| (1,7-dihydro-6 H-purine-6-thione | | |
| monohydrate) | | |
| mesna | Mesnex ™ | Asta Medica |
| (sodium 2-mercaptoethane sulfonate) | | |
| methotrexate | Methotrexate ™ | Lederle |
| (N-[4-[[(2,4-diamino-6- | | Laboratories |
| pteridinyl)methyl]methylamino]benzoyl]-L- | | |
| glutamic acid) | | |
| methoxsalen | Uvadex ™ | Therakos, Inc., Way |
| (9-methoxy-7H-furo[3,2-g][1]-benzopyran-7- | | Exton, Pa |
| one) | | |
| mitomycin C | Mutamycin ™ | Bristol-Myers |
| | | Squibb |
| mitomycin C | Mitozytrex ™ | SuperGen, Inc., |
| | | Dublin, CA |
| mitotane | Lysodren ™ | Bristol-Myers |
| (1,1-dichloro-2-(o-chlorophenyl)-2-(p- | | Squibb |
| chlorophenyl) ethane) | | |
| mitoxantrone | Novantrone ™ | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| nandrolone phenpropionate | Durabolin-50 ™ | Organon, Inc., West |
| | | Orange, NJ |
| nofetumomab | Verluma ™ | Boehringer |
| | | Ingelheim Pharma |
| | | KG, Germany |
| oprelvekin | Neumega ™ | Genetics Institute, |
| (IL-11) | | Inc., Alexandria, |
| | | VA |
| pamidronate | Aredia ™ | Novartis |
| (phosphonic acid (3-amino-1- | | |
| hydroxypropylidene) bis-, disodium salt, | | |
| pentahydrate, (APD)) | | |
| pegademase | Adagen ™ | Enzon |
| ((monomethoxypolyethylene glycol | (Pegademase | Pharmaceuticals, |
| succinimidyl) 11-17-adenosine deaminase) | Bovine) | Inc., Bridgewater, |
| | | NJ |
| pegaspargase | Oncaspar ™ | Enzon |
| (monomethoxypolyethylene glycol | | |
| succinimidyl L-asparaginase) | | |
| pegfilgrastim | Neulasta ™ | Amgen, Inc |
| (covalent conjugate of recombinant methionyl | | |
| human G-CSF (Filgrastim) and | | |
| monomethoxypolyethylene glycol) | | |
| pentostatin | Nipent ™ | Parke-Davis |
| | | Pharmaceutical Co., |
| | | Rockville, MD |
| pipobroman | Vercyte ™ | Abbott |
| | | Laboratories, |
| | | Abbott Park, IL |
| plicamycin, mithramycin | Mithracin ™ | Pfizer, Inc., NY, NY |
| (antibiotic produced by Streptomyces plicatus) | | |
| quinacrine | Atabrine ™ | Abbott Labs |
| (6-chloro-9-(1-methyl-4-diethyl-amine) | | |
| butylamino-2-methoxyacridine) | | |
| rasburicase | Elitek ™ | Sanofi-Synthelabo, |
| (recombinant peptide) | | Inc., |
| sargramostim | Prokine ™ | Immunex Corp |
| (recombinant peptide) | | |
| streptozocin | Zanosar ™ | Pharmacia & |
| (streptozocin 2-deoxy-2- | | Upjohn Company |
| [[(methylnitrosoamino)carbonyl]amino]- | | |
| a(and b)-D-glucopyranose and 220 mg citric | | |
| acid anhydrous) | | |
| talc | Sclerosol ™ | Bryan, Corp., |
| (Mg₃Si₄O₁₀ (OH)₂) | | Woburn, MA |
| temozolomide | Temodar ™ | Schering |
| (3,4-dihydro-3-methyl-4-oxoimidazo[5,1-d]- | | |
| as-tetrazine-8-carboxamide) | | |
| teniposide, VM-26 | Vumon ™ | Bristol-Myers |
| (4′-demethylepipodophyllotoxin9-[4,6-0-(R)- | | Squibb |
| 2-thenylidene-(beta)-D-glucopyranoside]) | | |
| testolactone | Teslac ™ | Bristol-Myers |
| (13-hydroxy-3-oxo-13,17-secoandrosta-1,4- | | Squibb |
| dien-17-oic acid [dgr ]-lactone) | | |
| thioguanine, 6-TG | Thioguanine ™ | GlaxoSmithKline |
| (2-amino-1,7-dihydro-6 H-purine-6-thione) | | |
| thiotepa | Thioplex ™ | Immunex |
| (Aziridine,1,1′,1″-phosphinothioylidynetris-, | | Corporation |
| or Tris (1-aziridinyl) phosphine sulfide) | | |
| topotecan HCl | Hycamtin ™ | GlaxoSmithKline |
| ((S)-10-[(dimethylamino) methyl]-4-ethyl-4,9- | | |
| dihydroxy-1H-pyrano[3′,4′:6,7] indolizino | | |
| [1,2-b] quinoline-3,14-(4H,12H)-dione | | |
| monohydrochloride) | | |
| toremifene | Fareston ™ | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, NJ |
| tositumomab, I 131 tositumomab | Bexxar ™ | Corixa Corp., |
| (recombinant murine immunotherapeutic | | Seattle, WA |
| monoclonal IgG₂ₐ lambda anti-CD20 antibody | | |
| (I 131 is a radioimmunotherapeutic antibody)) | | |
| tretinoin, ATRA | Vesanoid ™ | Roche |
| (all-trans retinoic acid) | | |
| uracil mustard | Uracil Mustard | Roberts Labs |
| | Capsules ™ | |
| valrubicin, N-trifluoroacetyladriamycin-14-valerate | Valstar ™ | Anthra --> Medeva |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| zoledronate, zoledronic acid | Zometa ™ | Novartis |
| ((1-Hydroxy-2-imidazol-1-yl-phosphonoethyl) | | |
| phosphonic acid monohydrate) | | |

23. Other Chemotherapeutic Agents

Additional drugs that may be administered or co-administered with compounds of the present invention include metformin, insulin, 2-deoxyglucose, sulfonylureas, anti-diabetic agents generally, mitochondrial oxidative-phoshorylation uncoupling agents, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), mTOR inhibitors, or agents that influence cancer metabolism.

24. Drug Cocktails

Chemotherapy agents can include cocktails of two or more chemotherapy drugs mentioned above. In several embodiments, a chemotherapy agent is a cocktail that includes two or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, oligomers or combinations thereof.

In one embodiment, the chemotherapy agent is a cocktail that includes an immunotherapy, an alkylating agent, an anthracycline, a camptothecin and prednisone. In other embodiments, the chemotherapy agent is a cocktail that includes rituximab, an alkylating agent, an anthracycline, a camptothecin and prednisone. In other embodiments, the chemotherapy agent is a cocktail that includes rituximab, cyclophosphamide, an anthracycline, a camptothecin and prednisone. In still other embodiments, the chemotherapy agent is a cocktail that includes rituximab, cyclophosphamide, doxorubicin, vincristine and prednisone (e.g., R-CHOP).

In another embodiment, the chemotherapy agent is a cocktail that includes doxorubicin, ifosfamide and mesna.

In other embodiments, the chemotherapy agent is a cocktail that includes an anti-metabolite and a taxane. For example, the chemotherapy agent includes gemcitabine and taxotere.

In other embodiments, the chemotherapy agent is a cocktail that includes dacarbazine, mitomycin, doxorubicin and cisplatin.

In other embodiments, the chemotherapy agent is a cocktail that includes doxorubicin and dacarbazine.

In alternative embodiments, the chemotherapy agent is a cocktail that includes an alkylating agent, a camptothecins, an anthracycline and dacarbazine. In other examples, the chemotherapy agent includes cyclophosphamide, vincristine, doxorubicin and dacarbazine.

In still other embodiments, the chemotherapy agent is a cocktail that includes an alkylating agent, methotrexate, an anti-metabolite and one or more anthracyclines. For example, the chemotherapy agent includes 5-fluorouracil, methotrexate, cyclophosphamide, doxorubicin and epirubicin.

In yet other embodiments, the chemotherapy agent is a cocktail that includes a taxane and prednisone or estramustine. For example, the chemotherapy agent can include docetaxel combined with prednisone or estramustine.

In still yet another embodiment, the chemotherapy agent includes an anthracycline and prednisone. For example, the chemotherapy agent can include mitoxantrone and prednisone.

In other embodiments, the chemotherapy agent includes a rapamycin macrolide and a kinase inhibitor. The kinase inhibitors can be EGFR, Her2/neu, VEGF, Aurora kinase, SRC/Abl kinase, tyrosine kinase, MET, and/or MEK inhibitors.

In another embodiment the chemotherapy agent includes two or more sphingolipid modulators.

In still another embodiment the chemotherapy agent includes an oligomer, such as Genasense® and one or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, PARP inhibitors or combinations thereof.

Moreover, the chemotherapy drug or drugs composing the chemotherapy agent can be administered in combination therapies with other agents, or they may be administered sequentially or concurrently to the patient.

b. Radiation Therapy

In several embodiments of the present invention, radiation therapy is administered in addition to the administration of an oligonucleotide compound. Radiation therapy includes both external and internal radiation therapies.

1. External Radiation Therapy

External radiation therapies include directing high-energy rays (e.g., x-rays, gamma rays, and the like) or particles (alpha particles, beta particles, protons, neutrons and the like) at the cancer and the normal tissue surrounding it. The radiation is produced outside the patient's body in a machine called a linear accelerator. External radiation therapies can be combined with chemotherapies, surgery or oligonucleotide compounds.

2. Internal Radiation Therapy

Internal radiation therapies include placing the source of the high-energy rays inside the body, as close as possible to the cancer cells. Internal radiation therapies can be combined with external radiation therapies, chemotherapies or surgery.

Radiation therapy can be administered with chemotherapy simultaneously, concurrently, or separately. Moreover radiation therapy can be administered with surgery simultaneously, concurrently, or separately.

c. Surgery

In alternative embodiments, of the present invention, surgery is used to remove cancerous tissue from a patient. Cancerous tissue can be excised from a patient using any suitable surgical procedure including, for example, laparoscopy, scalpel, laser, scissors and the like. In several embodiments, surgery is combined with chemotherapy. In other embodiments, surgery is combined with radiation therapy. In still other embodiments, surgery is combined with both chemotherapy and radiation therapy.

### IV. Pharmaceutical Compositions

In one aspect of the present invention, a pharmaceutical composition comprises one or more oligonucleotide compounds and a chemotherapy agent. For example, a pharmaceutical composition comprises an oligonucleotide compound having SEQ. ID NO. 1250, 1251, 1252, or 1253; and one or more of an alkylating agent, a platinum, an anti-metabolite, an anthracycline, a taxane, a camptothecins, a nitrosourea, an EGFR inhibitor, an antibiotic, a HER2/neu inhibitor, an angiogenesis inhibitor, a proteosome inhibitor, an immunotherapy, a hormone therapy, a photodynamic therapy, a cancer vaccine, a PARP inhibitor, a cell proliferation inhibitor, other chemotherapy agents such as those illustrated in Table 1, or combinations thereof.

In one embodiment, the pharmaceutical composition comprises an oligonucleotide compound and a chemotherapy agent including a dacarbazine, a B-RAF V600E inhibitor, or an antibody that binds to the cytotoxic T lymphocyte-associated antigen 4 (CTLA-4) or combinations thereof. The B-raf inhibitor may be vemurafenib. The CTLA-4 antibody may be ipilimumab.

The pharmaceutical composition may further comprise an immunotherapy, an alkylating agent, an anthracycline, a camptothecin and prednisone. For example, the pharmaceutical composition comprises one or more oligonucleotide compounds comprising SEQ ID NOs 2-281, 283-461, 463-935, 937-1080, 1082-1248, 1250-1254 and 1267-1477, and complements thereof; and a chemotherapy agent including an immunotherapy, an alkylating agent, an anthracycline, a camptothecin, and prednisone. In other embodiments, the pharmaceutical composition comprises an oligonucleotide compound and a chemotherapy agent that includes rituximab, cyclophosphamide, an anthracycline, a camptothecin and prednisone. In still other embodiments, the pharmaceutical composition comprises an oligonucleotide and a chemotherapy agent including rituximab, cyclophosphamide, doxorubicin, vincristine and prednisone (e.g., R-CHOP). In some embodiments, the pharmaceutical composition may comprise, for example, an oligonucleotide compound and bendamustine. In other embodiments, the pharmaceutical composition may comprise an oligonucleotide compound and fludarabine, cyclophosphamine, and, optionally, rituximab (FCR)

Pharmaceutical compositions of the present invention can optionally include medicaments such as anesthesia, nutritional supplements (e.g., vitamins, minerals, protein and the like), chromophores, combinations thereof, and the like.

A. Oligonucleotide Delivery

The oligonucleotide compounds of the present invention may be delivered using any suitable method. In some embodiments, naked DNA is administered. In other embodiments, lipofection is utilized for the delivery of nucleic acids to a subject. In still further embodiments, oligonucleotides are modified with phosphothiolates for delivery (See e.g., U.S. Pat. No. 6,169,177, herein incorporated by reference).

In some embodiments, oligonucleotides are sequestered in lipids (e.g., liposomes or micelles) to aid in delivery (See e.g., U.S. Pat. Nos. 6,458,382, 6,429,200; U.S. Patent Publications 2003/0099697, 2004/0120997, 2004/0131666, 2005/0164963, and International Publication WO 06/048329, each of which is herein incorporated by reference).

As used herein, “liposome” refers to one or more lipids forming a complex, usually surrounded by an aqueous solution. Liposomes are generally spherical structures comprising lipids, such as phospholipids, steroids, fatty acids, and are lipid bilayer type structures, and can include unilamellar vesicles, multilamellar structures, and amorphous lipid vesicles. Generally, liposomes are completely closed lipid bilayer membranes containing an entrapped aqueous volume. The liposomes may be unilamellar vesicles (possessing a single bilayer membrane) or multilamellar (onion-like structures characterized by multiple membrane bilayers, each separated from the next by an aqueous layer). Liposomes of the present invention may also include a DNAi oligonucleotide as defined below, either bound to the liposomes or sequestered in or on the liposomes. The molecules include, but are not limited to, DNAi oligonucleotides and/or other agents used to treat diseases such as cancer.

As used herein, “sequestered”, “sequestering”, or “sequester” refers to encapsulation, incorporation, or association of a drug, molecule, compound, including a DNAi oligonucleotide, with the lipids of a liposome. The molecule may be associated with the lipid bilayer or present in the aqueous interior of the liposome or both. “Sequestered” includes encapsulation in the aqueous core of the liposome. It also encompasses situations in which part or all of the molecule is located in the aqueous core of the liposome and part outside of the liposome in the aqueous phase of the liposomal suspension, where part of the molecule is located in the aqueous core of the liposome and part in the lipid portion of the liposome, or part sticking out of the liposomal exterior, where molecules are partially or totally embedded in the lipid portion of the liposome, and includes molecules associated with the liposomes, with all or part of the molecule associated with the exterior of the liposome.

Particularly, after a systemic application, the oligonucleotide and/or other agents must be stably sequestered in the liposomes until eventual uptake in the target tissue or cells. Accordingly, the guidelines for liposomal formulations of the FDA regulate specific preclinical tests for liposomal drugs (http://www.fda.gov/cder/guidance/2191dft.pdf). After injection of liposomes into the blood stream, serum components interact with the liposomes, which can lead to permeabilization of the liposomes. However, release of a drug or molecule that is encapsulated in a liposome depends on molecular dimensions of the drug or molecule. Consequently, a plasmid of thousands of base pairs is released much more slowly than smaller oligonucleotides or other small molecules. For liposomal delivery of drugs or molecules, it is ideal that the release of the drug during circulation of the liposomes in the bloodstream be as low as possible.

1. Amphoteric Liposomes

In some embodiments, liposomes used for delivery may be amphoteric liposomes, such as those described in US 2009/0220584, incorporated herein by reference. Amphoteric liposomes are a class of liposomes having anionic or neutral charge at about pH 7.5 and cationic charge at pH 4. Lipid components of amphoteric liposomes may be themselves amphoteric, and/or may consist of a mixture of anionic, cationic, and in some cases, neutral species, such that the liposome is amphoteric.

As used herein, an “amphoteric liposome” is a liposome with an amphoteric character, as defined below.

As used herein, sequestered, sequestering, or sequester refers to encapsulation, incorporation, or association of a drug, molecule, compound, including a DNAi oligonucleotide, with the lipids of a liposome. The molecule may be associated with the lipid bilayer or present in the aqueous interior of the liposome or both. “Sequestered” includes encapsulation in the aqueous core of the liposome. It also encompasses situations in which part or all of the molecule is located in the aqueous core of the liposome and part outside of the liposome in the aqueous phase of the liposomal suspension, where part of the molecule is located in the aqueous core of the liposome and part in the lipid portion of the liposome, or part sticking out of the liposomal exterior, where molecules are partially or totally embedded in the lipid portion of the liposome, and includes molecules associated with the liposomes, with all or part of the molecule associated with the exterior of the liposome.

As used herein, “polydispersity index” is a measure of the heterogeneity of the particle dispersion (heterogeneity of the diameter of liposomes in a mixture) of the liposomes. A polydispersity index can range from 0.0 (homogeneous) to 1.0 (heterogeneous) for the size distribution of liposomal formulations.

The amphoteric liposomes include one or more amphoteric lipids or alternatively a mix of lipid components with amphoteric properties. Suitable amphoteric lipids are disclosed in PCT International Publication Number WO02/066489 as well as in PCT International Publication Number WO03/070735, the contents of both of which are incorporated herein by reference. Alternatively, the lipid phase may be formulated using pH-responsive anionic and/or cationic components, as disclosed in PCT International Publication Number WO02/066012, the contents of which are incorporated by reference herein. Cationic lipids sensitive to pH are disclosed in PCT International Publication Numbers WO02/066489 and WO03/070220, in Budker, et al. 1996, Nat. Biotechnol., 14(6):760-4, and in U.S. Pat. No. 6,258,792 the contents of which are incorporated by reference herein, and can be used in combination with constitutively charged anionic lipids or with anionic lipids that are sensitive to pH. Conversely, the cationic charge may also be introduced from constitutively charged lipids that are known to those skilled in the art in combination with a pH sensitive anionic lipid. (See also PCT International Publication Numbers WO05/094783, WO03/070735, WO04/00928, WO06/48329, WO06/053646, WO06/002991 and U.S. Patent publications 2003/0099697, 2005/0164963, 2004/0120997, 2006/159737, 2006/0216343, each of which is also incorporated in its entirety by reference.)

Amphoteric liposomes of the present invention include 1) amphoteric lipids or a mixture of lipid components with amphoteric properties, (2) neutral lipids, (3) one or more DNAi oligonucleotides, (4) a cryoprotectant and/or lyoprotectant, and (5) a spray-drying cryoprotectant. In addition, the DNAi-liposomes have a defined size distribution and polydispersity index.

As used herein, “amphoter” or “amphoteric” character refers to a structure, being a single substance (e.g., a compound) or a mixture of substances (e.g., a mixture of two or more compounds) or a supramolecular complex (e.g., a liposome) comprising charged groups of both anionic and cationic character wherein
(i) at least one of the charged groups has a pK between 4 and 8,
(ii) the cationic charge prevails at pH 4 and
(iii) the anionic charge prevails at pH 8,
   resulting in an isoelectric point of neutral net charge between pH 4 and pH 8. Amphoteric character by that definition is different from zwitterionic character, as zwitterions do not have a pK in the range mentioned above. Consequently, zwitterions are essentially neutrally charged over a range of pH values. Phosphatidylcholine or phosphatidylethanolamines are neutral lipids with zwitterionic character.

As used herein, “Amphoter I Lipid Pairs” refers to lipid pairs containing a stable cation and a chargeable anion. Examples include without limitation DDAB/CHEMS, DOTAP/CHEMS and DOTAP/DOPS. In some aspects, the ratio of the percent of cationic lipids to anionic lipids is lower than 1.

As used herein, “Amphoter II Lipid Pairs” refers to lipid pairs containing a chargeable cation and a chargeable anion. Examples include without limitation Mo-Chol/CHEMS, DPIM/CHEMS or DPIM/DG-Succ. In some aspects, the ratio of the percent of cationic lipids to anionic lipids is between about 5 and 0.2.

As used herein, “Amphoter III Lipid Pairs” refers to lipid pairs containing a chargeable cation and stable anion. Examples include without limitation Mo-Chol/DOPG or Mo-Chol/Chol-SO₄. In one embodiment, the ratio of the percent of cationic lipids to anionic lipids is higher than 1.

Abbreviations for lipids refer primarily to standard use in the literature and are included here as a helpful reference:
DMPC Dimyristoylphosphatidylcholine
DPPC Dipalmitoylphosphatidylcholine
DSPC Distearoylphosphatidylcholine
POPC Palmitoyl-oleoylphosphatidylcholine
OPPC 1-oleoyl-2-palmitoyl-sn-glycero-3-phosphocholine
DOPC Dioleoylphosphatidylcholine
DOPE Dioleoylphosphatidylethanolamine
DMPE Dimyristoylphosphatidylethanolamine
DPPE Dipalmitoylphosphatidylethanolamine
DOPG Dioleoylphosphatidylglycerol
POPG Palmitoyl-oleoylphosphatidylglycerol
DMPG Dimyristoylphosphatidylglycerol
DPPG Dipalmitoylphosphatidylglycerol
DLPG Dilaurylphosphatidylglycerol
DSPG Distearoylphosphatidylglycerol
DMPS Dimyristoylphosphatidylserine
DPPS Dipalmitoylphosphatidylserine
DOPS Dioleoylphosphatidylserine
POPS Palmitoyl-oleoylphosphatidylserine
DMPA Dimyristoylphosphatidic acid
DPPA Dipalmitoylphosphatidic acid
DSPA Distearoylphosphatidic acid
DLPA Dilaurylphosphatidic acid
DOPA Dioleoylphosphatidic acid
POPA Palmitoyl-oleoylphosphatidic acid
CHEMS Cholesterolhemisuccinate
DC-Chol 3-β-[N—(N′,N′-dimethylethane) carbamoyl]cholesterol
Cet-P Cetylphosphate
DODAP (1,2)-dioleoyloxypropyl)-N,N-dimethylammonium chloride
DOEPC 1,2-dioleoyl-sn-glycero-3-ethylphosphocholine
DAC-Chol 3-β-[N—(N,N′-dimethylethane) carbamoyl]cholesterol
TC-Chol 3-β-[N—(N′,N′,N′-trimethylaminoethane) carbamoyl]cholesterol
DOTMA (1,2-dioleyloxypropyl)-N,N,N-trimethylammoniumchloride) (Lipofectin®)
DOGS ((C18)2GlySper3+) N,N-dioctadecylamido-glycyl-spermine (Transfectam®)
CTAB Cetyl-trimethylammoniumbromide
CPyC Cetyl-pyridiniumchloride
DOTAP (1,2-dioleoyloxypropyl)-N,N,N-trimethylammonium salt
DMTAP (1,2-dimyristoyloxypropyl)-N,N,N-trimethylammonium salt
DPTAP (1,2-dipalmitoyloxypropyl)-N,N,N-trimethylammonium salt
DOTMA (1,2-dioleyloxypropyl)-N,N,N-trimethylammonium chloride)
DORIE (1,2-dioleyloxypropyl)-3 dimethylhydroxyethyl ammoniumbromide)
DDAB Dimethyldioctadecylammonium bromide
DPIM 44-(2,3-bis-palmitoyloxy-propyl)-1-methyl-1H-imidazole
CHIM Histaminyl-Cholesterolcarbamate
MoChol 4-(2-Aminoethyl)-Morpholino-Cholesterolhemisuccinate
HisChol Histaminyl-Cholesterolhemisuccinate
HCChoI Nα-Histidinyl-Cholesterolcarbamate
HistChol Nα-Histidinyl-Cholesterol-hemisuccinate
AC Acylcarnosine, Stearyl- & Palmitoylcarnosine
HistDG 1,2-Dipalmitoylglycerol-hemisuccinat-N-Histidinyl-hemisuccinate; and Distearoyl-, Dimyristoyl-, Dioleoyl- or palmitoyl-oleoyl derivatives
IsoHistSuccDG 1,2-ipalmitoylglycerol-O-Histidinyl-Nα-hemisuccinate, and Distearoyl-, Dimyristoyl, Dioleoyl or palmitoyl-oleoyl derivatives
DGSucc 1,2-Dipalmitoyglycerol-3-hemisuccinate & Distearoyl-, dimyristoyl-Dioleoyl or palmitoyl-oleoylderivatives
EDTA-Chol cholesterol ester of ethylenediaminetetraacetic acid
Hist-PS Nα-histidinyl-phosphatidylserine
BGSC bisguanidinium-spermidine-cholesterol
BGTC bisguanidinium-tren-cholesterol
DOSPER (1,3-dioleoyloxy-2-(6-carboxy-spermyl)-propylarnide
DOSC (1,2-dioleoyl-3-succinyl-sn-glyceryl choline ester)
DOGSDO (1,2-dioleoyl-sn-glycero-3-succinyl-2-hydroxyethyl disulfide ornithine)
DOGSucc 1,2-Dioleoylglycerol-3-hemisucinate
POGSucc Palimtolyl-oleoylglycerol-oleoyl-3-hemisuccinate
DMGSucc 1,2-Dimyristoylglycerol-3-hemisuccinate
DPGSucc 1,2-Dipalmitoylglycerol-3-hemisuccinate

The following structures provide non-limiting examples of lipids that are suitable for use in the compositions in accordance with the present invention. The membrane anchors of the lipids are shown exemplarily and serve only to illustrate the lipids of the invention and are not intended to limit the same.

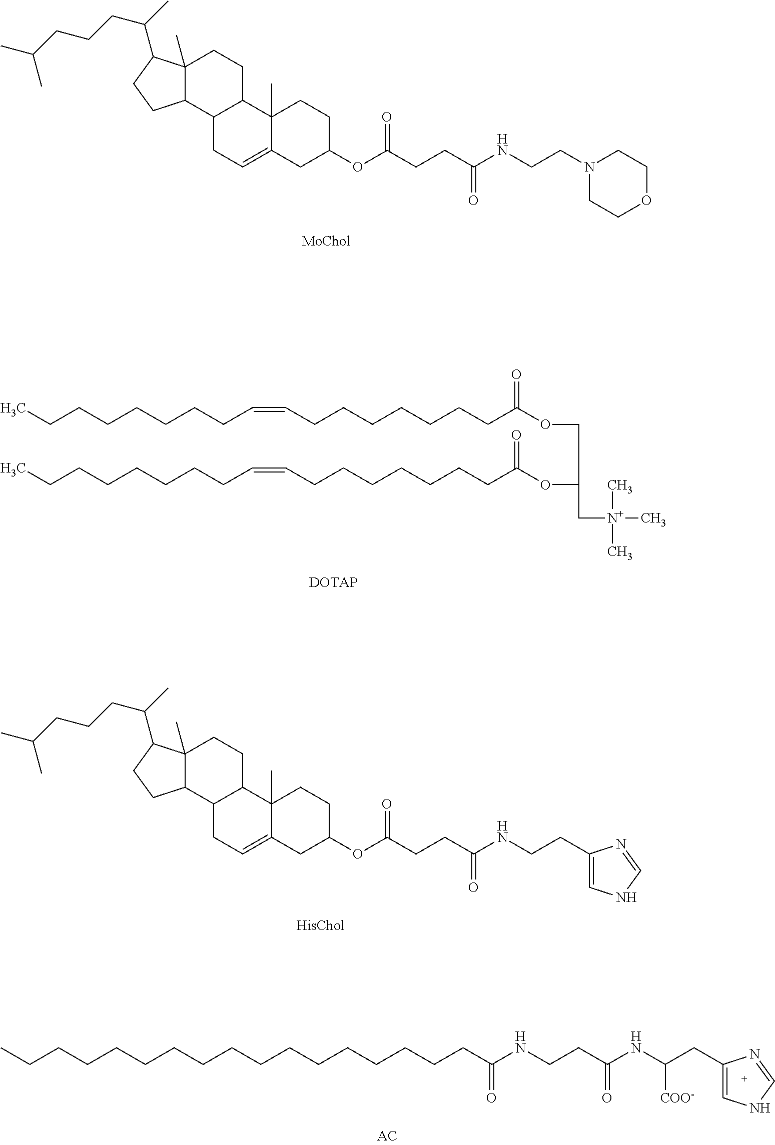

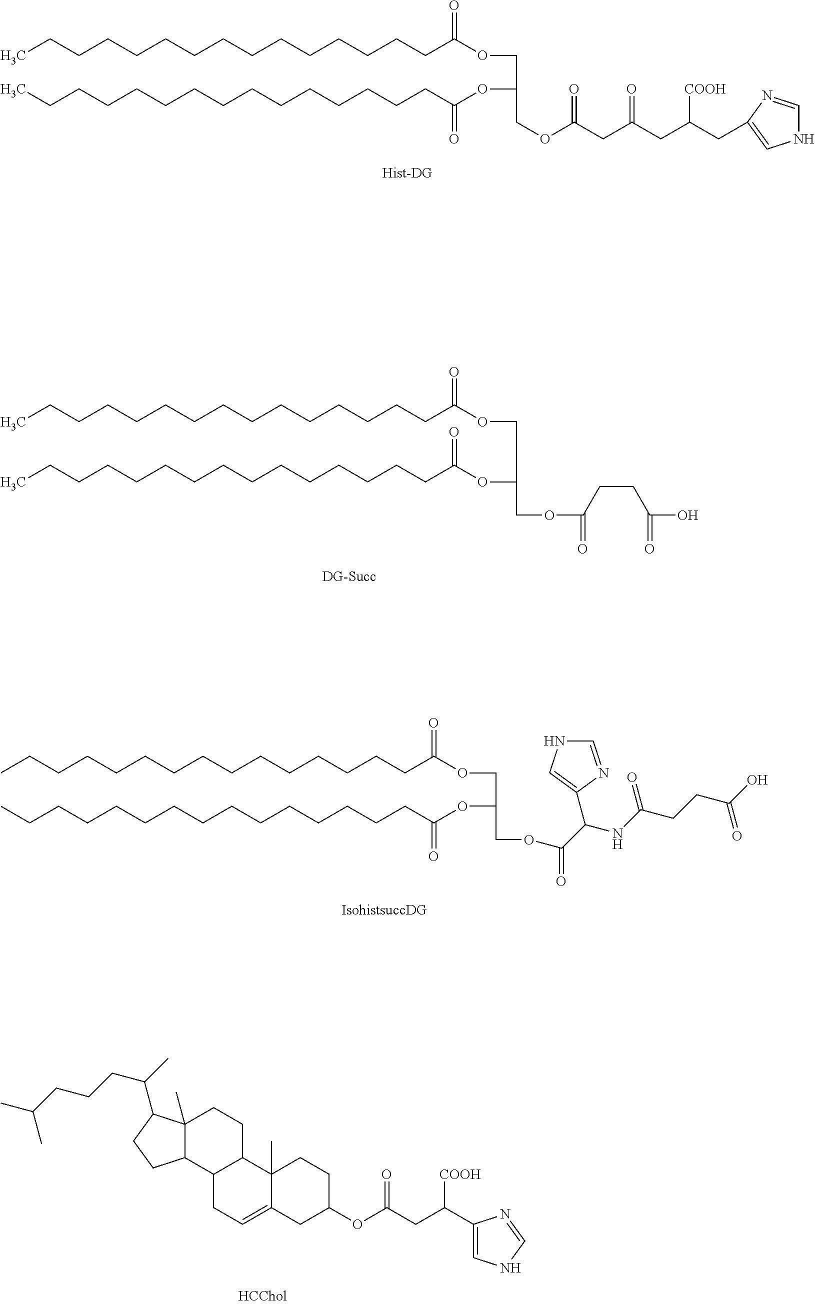 [SMILES: CCCCCCCCCCCCCCCC(=O)OCC(COC(=O)CC(=O)CC(Cc1c[nH]cn1)C(=O)O)OC(=O)CCCCCCCCCCCCCCC ; CCCCCCCCCCCCCCCC(=O)OCC(COC(=O)CCC(=O)O)OC(=O)CCCCCCCCCCCCCCC ; CCCCCCCCCCCCCCCC(=O)OCC(COC(=O)C(NC(=O)CCC(=O)O)c1c[nH]cn1)OC(=O)CCCCCCCCCCCCCCC ; CC(C)CCCC(C)C1CCC2C3CC=C4CC(OC(=O)CC(C(=O)O)c5c[nH]cn5)CCC4(C)C3CCC12C]

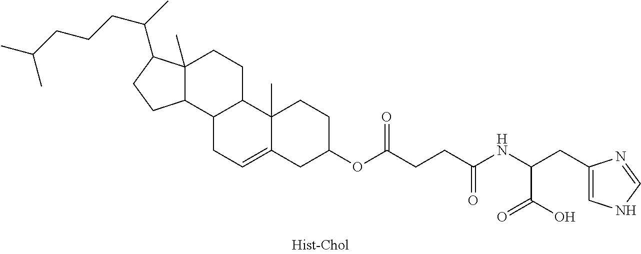 [SMILES: CC(C)CCCC(C)C1CCC2C3CC=C4CC(OC(=O)CCC(=O)NC(Cc5c[nH]cn5)C(=O)O)CCC4(C)C3CCC12C]

Amphoteric lipids are disclosed in PCT International Publication Numbers WO02/066489 and WO03/070735, the contents of both of which are incorporated herein by reference. The overall molecule assumes its pH-dependent charge characteristics by the simultaneous presence of cationic and anionic groups in the “amphoteric substance” molecule portion. More specifically, an amphoteric substance is characterized by the fact that the sum of its charge components will be precisely zero at a particular pH value. This point is referred to as isoelectric point (IP). Above the IP the compound has a negative charge, and below the IP it is to be regarded as a positive cation, the IP of the amphoteric lipids according to the invention ranging between 4.5 and 8.5.

The overall charge of the molecule at a particular pH value of the medium can be calculated as follows:

z=Σni×((qi−1)+(10^{(pK-pH)}/(1+10^{(pK-pH)}))
qi: absolute charge of the ionic group below the pK thereof (e.g. carboxyl=0, single-nitrogen base=1, di-esterified phosphate group=−1)
ni: number of such groups in the molecule.

For example, a compound is formed by coupling the amino group of histidine to cholesterol hemisuccinate. At a neutral pH value of 7, the product has a negative charge because the carboxyl function which is present therein is in its fully dissociated form, and the imidazole function only has low charge. At an acid pH value of about 4, the situation is reversed: the carboxyl function now is largely discharged, while the imidazole group is essentially fully protonated, and the overall charge of the molecule therefore is positive.

In one embodiment, the amphoteric lipid is selected from the group consisting of HistChol, HistDG, isoHistSuccDG, Acylcarnosine and HCChol. In another embodiment, the amphoteric lipid is HistChol.

Amphoteric lipids can include, without limitation, derivatives of cationic lipids which include an anionic substituent Amphoteric lipids include, without limitation, the compounds having the structure of the formula:

Z-X-W1-Y-W2-HET

wherein:

Z is a sterol or an aliphatic;

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5a-cholest-7-en-3β-ol, 7-hydroxycholesterol, epocholesterol, ergosterol dehydroergosterol, and derivatives thereof;

Each W1 is independently an unsubstituted aliphatic;

Each W2 is independently an aliphatic optionally substituted with HO(O)C-aliphatic-amino or carboxy;

Each X and Y is independently absent, —(C═O)—O—, —(C═O)—NH—, —(C═O)—S—, —O—, —NH—, —S—, —CH═N—, —O—(O═C)—, —S—(O═C)—, —NH—(O═C)—, —N═CH—, and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl, or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

In other embodiments, amphoteric lipids include, without limitation, the compounds having the structure of the formula:

Z—X—W1-Y—W2-HET

wherein:

Z is a structure according to the general formula

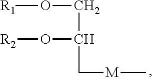

wherein R1 and R2 are independently C₅-C₃₀ alkyl or acyl chains with 0, 1 or 2 ethylenically unsaturated bonds and M is selected from the group consisting of —O—(C═O); —NH—(C═O)—; —S—(C═O)—; —O—; —NH—; —S—; —N═CH—; —(O═C)—O—; —S—(O═C)—; —NH—(O═C)—, —N═CH—, —S—S—; and

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5αcholest-7-en-3β-ol, 7-hydroxycholesterol, epicholesterol, ergosterol dehydroergosterol, and derivatives thereof;

Each W1 is independently an unsubstituted aliphatic with up to 8 carbon atoms;

Each W2 is independently an aliphatic, carboxylic acid with up to 8 carbon atoms and 0, 1, or 2 ethyleneically unsaturated bonds;

X is absent and Y is —(C═O)—O—; —(C═O)—NH—; —NH—(C═O)—O—; —O—; —NH—; —CH═N—; —O—(O═C)—; —S—; —(O═C)—; —NH—(O═C)—; —O—(O═C)—NH—, —N═CH— and/or —S—S—; and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl, or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

Alternatively, the lipid phase can be formulated using pH-responsive anionic and/or cationic components, as disclosed in PCT International Publication Number WO02/066012, the contents of which are incorporated by reference herein. Cationic lipids sensitive to pH are disclosed in PCT International Publication Numbers WO02/066489 and WO03/070220, in Budker, et al. (1996), Nat Biotechnol. 14(6):760-4, and in U.S. Pat. No. 6,258,792, the contents of all of which are incorporated by reference herein. Alternatively, the cationic charge may be introduced from constitutively charged lipids known to those skilled in the art in combination with a pH sensitive anionic lipid. Combinations of constitutively (e.g., stable charge over a specific pH range such as a pH between about 4 and 9) charged anionic and cationic lipids, e.g. DOTAP and DPPG are not preferred. Thus, in some embodiments of the invention, the mixture of lipid components may comprise (i) a stable cationic lipid and a chargeable anionic lipid, (ii) a chargeable cationic lipid and chargeable anionic lipid or (iii) a stable anionic lipid and a chargeable cationic lipid.

The charged groups can be divided into the following 4 groups.

(1) Strongly (e.g., constitutively charged) cationic, pKa>9, net positive charge: on the basis of their chemical nature, these are, for example, ammonium, amidinium, guanidium or pyridinium groups or timely, secondary or tertiary amino functions.

(2) Weakly cationic, pKa<9, net positive charge: on the basis of their chemical nature, these are, in particular, nitrogen bases such as piperazines, imidazoles and morpholines, purines or pyrimidines. Such molecular fragments, which occur in biological systems, are, for example, 4-imidazoles (histamine), 2-, 6-, or 9-purines (adenines, guanines, adenosines or guanosines), 1-, 2- or 4-pyrimidines (uracils, thymines, cytosines, uridines, thymidines, cytidines) or also pyridine-3-carboxylic acids (nicotinic esters or amides). Nitrogen bases with preferred pKa values are also formed by substituting nitrogen atoms one or more times with low molecular weight alkene hydroxyls, such as hydroxymethyl or hydroxyethyl groups. For example, aminodihydroxypropanes, triethanolamines, tris-(hydroxymethyl)methylamines, bis-(hydroxymethyl)methylamines, tris-(hydroxyethyl)methylamines, bis-(hydroxyethyl)methylamines or the corresponding substituted ethylamines.

(3) Weakly anionic, pKa>4, net negative charge: on the basis of their chemical nature, these are, in particular, the carboxylic acids. These include the aliphatic, linear or branched mono-, di- or tricarboxylic acids with up to 12 carbon atoms and 0, 1 or 2 ethylenically unsaturated bonds. Carboxylic acids of suitable behavior are also found as substitutes of aromatic systems. Other weakly anionic groups are hydroxyls or thiols, which can dissociate and occur in ascorbic acid, N-substituted alloxane, N-substituted barbituric acid, veronal, phenol or as a thiol group.

(4) Strongly (e.g., constitutively charged) anionic, pKa<4, net negative charge: on the basis of their chemical nature, these are functional groups such as sulfonate or phosphate esters.

The amphoteric liposomes contain variable amounts of such membrane-forming or membrane-based amphiphilic materials, so that they have an amphoteric character. This means that the liposomes can change the sign of the charge completely. The amount of charge carrier of a liposome, present at a given pH of the medium, can be calculated using the following formula:

z=Σni((qi−1)+10^{(pK-pH)}/(1+10^{(pK-pH)})

in which
qi is the absolute charge of the individual ionic groups below their pK (for example, carboxyl=0, simple nitrogen base=1, phosphate group of the second dissociation step=−1, etc.)
ni is the number of these groups in the liposome.

At the isoelectric point, the net charge of the liposome is 0. Structures with a largely selectable isoelectric point can be produced by mixing anionic and cationic portions.

In one embodiment, cationic components include DPIM, CHIM, DORIE, DDAB, DAC-Chol, TC-Chol, DOTMA, DOGS, (C18)₂Gly⁺N,N-dioctadecylamido-glycine, CTAB, CPyC, DODAP DMTAP, DPTAP, DOTAP, DC-Chol, MoChol, HisChol and DOEPC. In another embodiment, cationic lipids include DMTAP, DPTAP, DOTAP, DC-Chol, MoChol and HisChol.

The cationic lipids can be compounds having the structure of the formula

L-X-spacer1-Y-spacer2-HET

wherein:

L is a sterol or [aliphatic(C(O)O)-]₂-alkyl-; Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5acholest-7-en-30-ol, 7-hydroxycholesterol, epocholesterol, ergosterol dehydroergosterol, and derivatives thereof;

Each spacer 1 and spacer 2 is independently an unsubstituted aliphatic;

Each X and Y is independently absent, —(C═O)—O—, —(C═O)—NH—, —(C═O)—S—, —O—, —NH—, —S—, —O—(O═C)—, —S—(O═C)—, —NH—(O═C)—, —N═CH—, and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

In another embodiment, pH sensitive cationic lipids can be compounds having the structure of the formula

L-X-spacer1-Y-spacer2-HET

wherein:

L is a structure according to the general formula

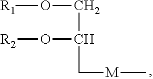

wherein R1 and R2 are independently C₈-C₃₀ alkyl or acyl chains with 0, 1 or 2 ethylenically unsaturated bonds and M is absent, —O—(C═O); —NH—(C═O)—; —S—(C═O)—; —O—; —NH—; —S—; —N═CH—; —(O═C)—O—; —S—(O═C)—; —NH—(O═C)—; —S—S—; and

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesterol, dihydrocholesterol, 19-hydroxycholesterol, 5α-cholest-7-en-3β-ol, 7-hydroxycholesterol, epicholesterol, ergosterol dehydroergosterol, and derivatives thereof;

Each spacer 1 and spacer 2 is independently an unsubstituted aliphatic with 1-8 carbon atoms;

X is absent and Y is absent, —(C═O)—O—; —(C═O)—NH—; —NH—(C═O)—O—; —O—; —NH—; —CH═N—; —O—(O═C)—; —S—; —(O═C)—; —NH—(O═C)—; —O—(O═C)—NH—, —N═CH— and/or —S—S—; and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

The above compounds can be synthesized using syntheses of 1 or more steps, and can be prepared by one skilled in the art.

The amphoteric mixtures further comprise anionic lipids, either constitutively or conditionally charged in response to pH, and such lipids are also known to those skilled in the art. In one embodiment, lipids for use with the invention include DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and CetylP. In another embodiment, anionic lipids include DOGSucc, DMGSucc, DMPG, DPPG, DOPG, POPG, DMPA, DPPA, DOPA, POPA, CHEMS and CetylP.

Neutral lipids include any lipid that remains neutrally charged at a pH between about 4 and 9. Neutral lipids include, without limitation, cholesterol, other sterols and derivatives thereof, phospholipids, and combinations thereof. The phospholipids include any one phospholipid or combination of phospholipids capable of forming liposomes. They include phosphatidylcholines, phosphatidylethanolamines, lecithin and fractions thereof, phosphatidic acids, phosphatidylglycerols, phosphatidylinolitols, phosphatidylserines, plasmalogens and sphingomyelins. The phosphatidylcholines include, without limitation, those obtained from egg, soy beans or other plant sources or those that are partially or wholly synthetic or of variable lipid chain length and unsaturation, POPC, OPPC, natural or hydrogenated soy bean PC, natural or hydrogenated egg PC, DMPC, DPPC, DSPC, DOPC and derivatives thereof. In one embodiment, phosphatidylcholines are POPC, non-hydrogenated soy bean PC and non-hydrogenated egg PC. Phosphatidylethanolamines include, without limitation, DOPE, DMPE and DPPE and derivatives thereof. Phosphatidylglycerols include, without limitation, DMPG, DLPG, DPPG, and DSPG. Phosphatidic acids include, without limitation, DSPA, DMPA, DLPA and DPPA.

Sterols include cholesterol derivatives such as 3-hydroxy-5,6-cholestene and related analogs, such as 3-amino-5,6-cholestene and 5,6-cholestene, cholestane, cholestanol and related analogs, such as 3-hydroxy-cholestane; and charged cholesterol derivatives such as cholesteryl-beta-alanine and cholesterol hemisuccinate. Sterols further include MoChol and analogues of MoChol.

In one embodiment neutral lipids include but are not limited to DOPE, POPC, soy bean PC or egg PC and cholesterol.

In some aspects, the invention provides a mixture comprising amphoteric liposomes and a DNAi oligonucleotide. In an embodiment of the first aspect, the amphoteric liposomes have an isoelectric point of between 4 and 8. In a further embodiment, the amphoteric liposomes are negatively charged or neutral at pH 7.4 and positively charged at pH 4.

In some embodiments, the amphoteric liposomes include amphoteric lipids. In a further embodiment, the amphoteric lipids can be HistChol, HistDG, isoHistSucc DG, Acylcamosine, HCChol or combinations thereof. In another embodiment, the amphoteric liposomes include a mixture of one or more cationic lipids and one or more anionic lipids. In yet another embodiment, the cationic lipids can be DMTAP, DPTAP, DOTAP, DC-Chol, MoChol or HisChol, or combinations thereof, and the anionic lipids can be CHEMS, DGSucc, Cet-P, DMGSucc, DOGSucc, POGSucc, DPGSucc, DG Succ, DMPS, DPPS, DOPS, POPS, DMPG, DPPG, DOPG, POPG, DMPA, DPPA, DOPA, POPA or combinations thereof.

In yet another embodiment, the liposomes also include neutral lipids. In a further embodiment, the neutral lipids include sterols and derivatives thereof. In an even further embodiment, the sterols comprise cholesterol and derivatives thereof. The neutral lipids may also include neutral phospholipids. In one embodiment, the phospholipids include phosphatidylcholines or phosphatidylcholines and phosphoethanolamines. In another embodiment, the phosphatidylcholines are POPC, OPPC, natural or hydrogenated soy bean PC, natural or hydrogenated egg PC, DMPC, DPPC or DOPC and derivatives thereof and the phosphatidylethanolamines are DOPE, DMPE, DPPE or derivatives and combinations thereof. In a further embodiment, the phosphatidylcholine is POPC, OPPC, soy bean PC or egg PC and the phosphatidylethanolamines is DOPE.

In an even further embodiment, the lipids of the amphoteric liposomes include DOPE, POPC, CHEMS and MoChol; POPC, Chol, CHEMS and DOTAP; POPC, Chol, Cet-P and MoChol, or POPC, DOPE, MoChol and DMGSucc.

In another aspect, the amphoteric liposomes of the mixture of the invention can be formed from a lipid phase comprising a mixture of lipid components with amphoteric properties, wherein the total amount of charged lipids in the liposome can vary from 5 mole % to 70 mole %, the total amount of neutral lipids may vary from 20 mole % to 70 mole %, and a DNAi oligonucleotide. In an embodiment of the first aspect, the amphoteric liposomes include 3 to 20 mole % of POPC, 10 to 60 mole % of DOPE, 10 to 60 mole % of MoChol and 10 to 50 mole % of CHEMS. Ina further embodiment, the liposomes include POPC, DOPE, MoChol and CHEMS in the molar ratios of POPC/DOPE/MoChol/CHEMS of about 6/24/47/23 or 15/45/20/20. In yet another embodiment, the liposomes include 3 to 20 mole % of POPC, 10 to 40 mole % of DOPE, 15 to 60 mole % of MoChol and 15 to 60 mole % of DMGSucc. In a further embodiment, the liposomes include POPC, DOPE, DMGSucc and MoChol in the molar ratios of POPC/DOPE/DMGSucc/MoChol of about 6/24/47/23 or 6/24/23/47. In still another embodiment, the liposomes include 10 to 50 mole % of POPC, 20 to 60 mole % of Chol, 10 to 40 mole % of CHEMS and 5 to 20 mole % of DOTAP. In a further embodiment, the liposomes include POPC, Chol, CHEMS and DOTAP in the molar ratio of POPC/Chol/CHEMS/DOTAP of about 30/40/20/10. In yet another embodiment the liposomes include 10 to 40 mole % of POPC, 20 to 50 mole % of Chol, 5 to 30 mole % of Cet-P and 10 to 40 mole % of MoChol. In a further embodiment, the molar ratio of POPC/Chol/Cet-P/MoChol is about 35/35/10/20.

In a third aspect, the DNAi oligonucleotide contained in the amphoteric liposomal mixture comprises a DNAi oligonucleotide that hybridizes to SEQ ID NO:1249 or portions thereof. In another embodiment, the DNAi oligonucleotide can be SEQ ID NO:1250, 1251, 1252, 1253, 1267-1447 or the complement thereof. In yet another embodiment the DNAi oligonucleotide can be SEQ ID NO:1250 or 1251 or the complement thereof.

The amphoteric liposomal mixture of this invention may further include an additional DNAi oligonucleotide, e.g., comprising one of SEQ ID NOs: 1250-1253 and 1270-1477, or selected from the group consisting of SEQ ID NOs: 2-281, 283-461, 463-935, 937-1080, 1082-1248 and the complements thereof.

In another aspect, the DNAi oligonucleotides contained in the liposomal mixture are between 15 and 35 base pairs in length.

In another aspect, the amphoteric liposome-DNAi oligonucleotide mixture includes the DNAi oligonucleotides SEQ ID NO:1250 or 1251 and amphoteric liposomes comprising POPC, DOPE, MoChol and CHEMS in the molar ratio of POPC/DOPE/MoChol/CHEMS of about 6/24/47/23.

In another aspect, the amphoteric liposome-DNAi oligonucleotide mixture includes the DNAi oligonucleotide, PNT-100 (SEQ ID NO:1250 or 1251), and amphoteric liposomes comprising POPC, DOPE, MoChol and CHEMS in the molar ratio of POPC/DOPE/MoChol/CHEMS of about 15/45/20/20.

In another aspect, the amphoteric liposomes of the mixture can include a size between 50 and 500 ηm. In one embodiment, the size is between 80 and 300 ηm and in another embodiment the size is between 90 and 200 ηM.

In another aspect, the amphoteric liposomes may have an isoelectric point between 4 and 8. In an embodiment of the sixth aspect, the amphoteric liposomes may be negatively charged or neutral at pH 7.4 and positively charged at pH 4.

In another aspect, the amphoteric liposomes have a DNAi oligonucleotide concentration of at least about 2 mg/ml at a lipid concentration of 10 to 100 mM or less.

In another aspect, the invention provides a method of preparing amphoteric liposomes containing a DNAi oligonucleotide. In one embodiment, the method includes using an active loading procedure and in another, a passive loading procedure. In a further embodiment, the method produces liposomes using manual extrusion, machine extrusion, homogenization, microfluidization or ethanol injection. In yet another embodiment, the method has an encapsulation efficiency of at least 35%.

In another aspect, the invention provides a method of introducing the DNAi oligonucleotide-amphoteric liposome mixture to cells or an animal. In one embodiment, the method includes administering the mixture to mammal to treat cancer. The administered mixtures can reduce or stop tumor growth in mammals. In another embodiment, the introduction of the mixture results in a reduction of cell proliferation. In another embodiment, the mixture is administered to a cancer cell, a non-human animal or a human. In a further embodiment, the mixture is introduced to an animal at a dosage of between 0.01 mg to 100 mg per kg of body weight. In yet another embodiment, the mixture is introduced to the animal one or more times per day or continuously. In still another embodiment, the mixture is introduced to the animal via topical, pulmonary or parenteral administration or via a medical device. In an even further embodiment, the mixture administered to the animal or cells further includes a chemotherapy agent, and/or a cell targeting component.

In some embodiments, amphoteric liposomes formulations may comprise POPC/DOPE/MoChol/CHEMS at molar ratios of 6/24/47/23, respectively. Such liposomes are are cholesterol-rich and negatively-charged. This is unique among lipid delivery systems and contributes to cellular uptake. In some embodiments, oligonucleotides of SEQ ID NO: 1251 (PNT-100) may be sequestered in amphoteric liposomes with this formulation (hereinafter, “PNT 2258”).

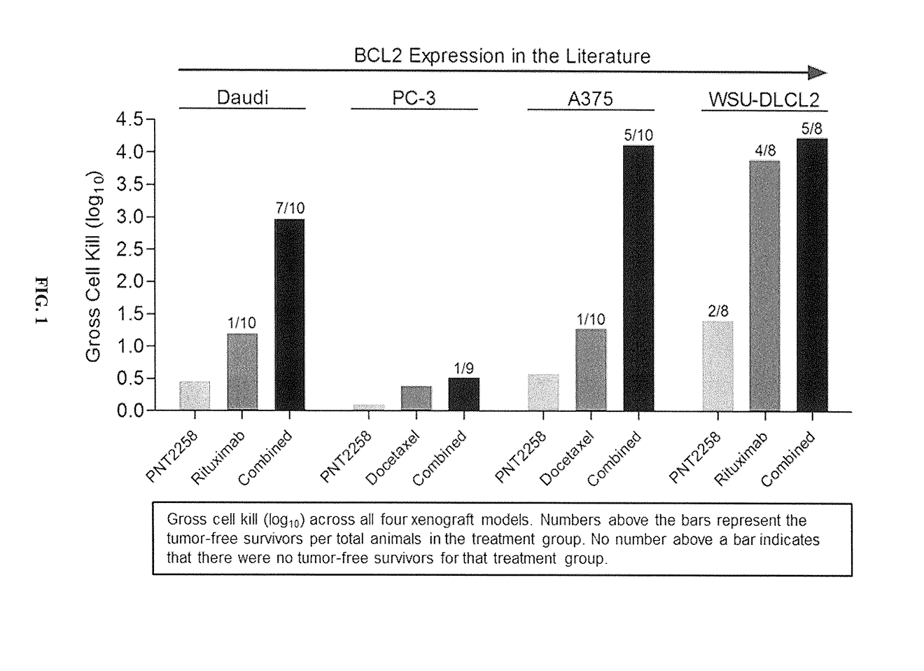

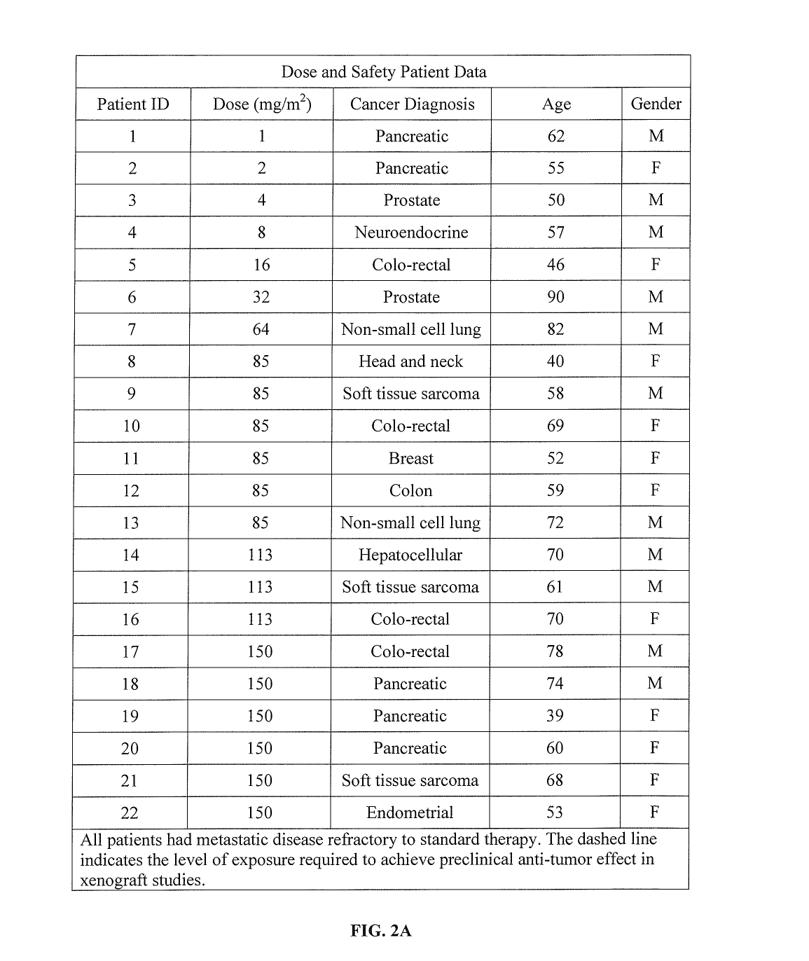

PNT2258, is an innovative therapeutic that is expected to address unmet medical needs in many cancers where the target gene BCL2 is overexpressed. It is known that BCL2 is overexpressed in lymphoma, leukemia, prostate, melanoma, sarcoma, lung, and breast cancers. PNT2258 showed anti-tumor activity against almost all of these indications in mouse models of cancer alone, as well as in combination with rituxamib or docetaxel (FIG. 1). In combination, PNT2258 demonstrated tumor-free survival in all the models. However, treatment of these and other tumors with PNT2258 in combinations with dacarbazine, Vemurafenib (PLX4032), or ipilimumab has not been tested before. Combinations of these agents are likely to have a statistically beneficial effect on tumor free progression or overall survival in humans suffering from cancer, including but not limited to metastatic melanoma.

PNT2258 is cholesterol-rich and negatively-charged. This is unique among lipid delivery systems and contributes to cellular uptake. PNT2258 has shown long circulating half-life, stability, and remarkable antitumor efficacy in animal models. It is also well established that rapidly dividing cells scavenge cholesterol from the circulation/intracellular milieu and cholesterol-rich particles are attracted to the extracellular matrix. Not to be limited by theory, it is postulated that PNT2258 is likely directed into cells through these mechanisms.

2. Other Liposomal Delivery Vehicles

Liposomes include, without limitation, cardiolipin based cationic liposomes (e.g., NeoPhectin, available from NeoPharm, Forest Lake, Ill.) and pH sensitive liposomes.

In some embodiments of the present invention, NeoPhectin is utilized as the liposomal delivery vehicle. In some embodiments, the NeoPhectin is formulated with the oligonucleotide so as to reduce free NeoPhectin. In other embodiments, NeoPhectin is present at a charge ratio 6:1 or less (e.g., 5:1, and 4:1) of NeoPhectin to oligonucleotide.

In yet other embodiments, lipids, particularly phospholipids that comprise some liposomes, are conjugated to polyethylene glycol or a derivative thereof, to increase the time that the liposomes circulate in the blood after intravenous injection. (See e.g., Moghimi, S. M. and Szebeni, J, Prog. Lipid Res., 42:463-78, 2003 and Li, W., et al., J. Gene Med., 7:67-79, 2005, which are incorporated herein by reference.) Such liposomes, termed “stealth liposomes” are able to avoid the reticuloentothelial system (RES), resulting in half lives of more than 24 hours in some cases. In one embodiment, the phospholipids in liposomes are conjugated to polyethylene glycol-diorthoester molecules, as described in Li, W., et al., J. Gene Med., 7:67-79, 2005. In other embodiments, the PEG-liposomes are targeted to specific cell receptors. For example, haloperidol conjugated at the distal end of a PEG-linked phospholipids in a cationic liposome targeted sigma receptors that are overexpressed on some cancer cells as described in Mukherjee, et al., J. Biol. Chem., 280, 15619-27, 2005, which is incorporated herein by reference. Anisamide conjugated to PEG-linked phospholipids in liposomes also targets the sigma receptor. (Banerjee, et al., Int. J. Cancer, 112, 693-700, 2004, which is incorporated herein by reference.)

Other liposomic delivery vehicles include lipid nanoparticles which are designed to encapsulate and deliver small oligonucleotides. Examples of lipid nanoparticles include, but are not limited to, for example, stable nucleic-acid-lipid particles (SNALPS; see e.g., Semple et al. Nature Biotech. Lett. (Jan. 17, 2010 doi:10.1038/nbt.1602); and lipidoids (see e.g., Love et al., P.N.A.S. (USA) 107(5) 1864-1869).

3. Polymeric Vesicles

In further embodiments, oligonucleotides are sequestered in polymer vesicles. Polymer vesicles can be made from a number of different materials, but in general are formed from block copolymers, for example, polystyrene₄₀-poly(isocyano-L-alanine-L-alanine)ₘ. (See for example, Discher, et al., Science, 297:967-73, 2002; Torchilin, Cell. Mol. Life Sci, 61:2549-59, 2004; Taubert, et al., Curr Opin Chem Biol, 8:598-603, 2004; Lee, et al., Pharm. Res., 22:1-10, 2005; and Gaucher, et al., J. Control. Rel, 109:169-88, 2005, each of which is incorporated herein by reference.) Copolymer vesicles are formed from a number of molecules, including, without limitation, polyacrylic acid-polystyrene, nonionic polyethyleneoxide-polybutadiene, the triblock (polyethyleneoxide)₅-(poly[propyleneoxide])₆₈-(polyethyleneoxide)₅, polyethyleneoxide-poly(propylenesulfide), polyethyleneoxide-polylactide, and polyethylene glycol-polylysine. Many copolymers, particularly those of either amphiphilic or oppositely charged copolymers, including polystyrene₄₀-poly(isocyano-L-alanine-L-alanine)ₘ, self assemble into vesicles in aqueous conditions.

Oligonucleotides can be loaded into the polymer vesicles using several methods. First, the block copolymer can be dissolved along with the oligonucleotides in an aqueous solvent. This method works well with moderately hydrophobic copolymers. Second, for amphiphilic copolymers that are not readily soluble in water, and where a solvent that solubilizes both the oligonucleotides and the copolymer is available, the oligonucleotide and copolymer are dissolved in the solvent and the mixture is dialyzed against water. A third method involves dissolving both the oligonucleotides and copolymer in a water/tert-butanol mixture and subsequent lyophilization of the solvents. The oligonucleotide-loaded vesicles are formed spontaneously when the lyophilized oligonucleotide-copolymer is reconstituted in an injectable vehicle. (Dufresne, et al., in Gurny, (ed.), B. T. Gattefosse, vol. 96, Gattefosse, Saint-Priest, p. 87-102, 2003, which is incorporated herein by reference.)

Polymer vesicles can be targeted to specific cells by tethering a ligand to the outer shell of vesicles by post modification of a copolymer with a bifunctional spacer molecule or by the direct synthesis of heterobifunctional block copolymers.

In yet another embodiment, oligonucleotides can be sequestered in hybrid liposome-copolymer vesicles, as described in Ruysschaert, et al., J. Am. Chem. Soc., 127, 6242-47, 2005, which is incorporated herein by reference. For example, an amphiphilic triblock copolymers, including poly(2-methyloxazoline)-block-poly(dimethylsiloxan)-block-poly(2-methyloxazoline) can interact with lipids, including phospholipids to form hybrid liposome-copolymer vesicles.

4. Oligonucleotide Modifications

In some embodiments, nucleic acids for delivery are compacted to aid in their uptake (See e.g., U.S. Pat. Nos. 6,008,366, 6,383,811 herein incorporated by reference). In some embodiments, compacted nucleic acids are targeted to a particular cell type (e.g., cancer cell) via a target cell binding moiety (See e.g., U.S. Pat. Nos. 5,844,107, 6,077,835, each of which is herein incorporated by reference).

In some embodiments, oligonucleotides are conjugated to other compounds to aid in their delivery. For example, in some embodiments, nucleic acids are conjugated to polyethylene glycol to aid in delivery (See e.g., U.S. Pat. Nos. 6,177,274, 6,287,591, 6,447,752, 6,447,753, and 6,440,743, each of which is herein incorporated by reference). In yet other embodiments, oligonucleotides are conjugated to protected graft copolymers, which are chargeable” drug nano-carriers (PharmaIn), described in U.S. Pat. No. 7,138,105, and U.S. publication numbers 2006/093660 and 2006/0239924, which are incorporated herein by reference. In still further embodiments, the transport of oligonucleotides into cells is facilitated by conjugation to vitamins (Endocyte, Inc, West Lafayette, Ind.; See e.g., U.S. Pat. Nos. 5,108,921, 5,416,016, 5,635,382, 6,291,673 and WO 02/085908; each of which is herein incorporated by reference). In other embodiments, oligonucleotides are conjugated to nanoparticles (e.g., NanoMed Pharmaceuticals; Kalamazoo, Mich.).

In still other embodiments, oligonucleotides are associated with dendrimers. Dendrimers are synthetic macromolecules with highly branched molecular structures. Representative dendrimeric structures are cationic polymers such as starburst polyamidoamine (PAMAM), one of which, SuperFect®, is available from Qiagen (Valencia, Calif.). Other dendrimers include polyester dentrimers described by Gillies, et al., Mol. Pharm., 2:129-38, 2005, which is incorporated herein by reference; phenylacetylene dendrimers, described in Janssen and Meijer, eds, Synthesis of Polymers, Materials science and technology series, Weinheim, Germany: Wiley-VCH Verlag GMBH, Chapter 12, 1999, which is incorporated herein by reference; poly(L-lysine) dendrimer-block-poly(ethylene glycol)-block-poly(L-lysine) dendrimers described by Choi, et al., J. Am. Chem. Soc. 122, 474-80, 2000, which is incorporated herein by reference; amphiphilic dendrimers, described by Joester, et al., Angew Chem Int. Ed. Engl., 42:1486-90, 2003, which is incorporated herein by reference; polyethylene glycol star like conjugates, described by Liu et al., Polym Chem, 37:3492-3503, 1999, which is incorporated herein by reference; cationic phosphorus-containing dendrimers described by Loup, et al., Chem Eur J, 5:3644-50, 1999, which is incorporated herein by reference; poly(L-lysine) dendrimers, described by Ohasaki, et al., Bioconjug Chem, 13:510-17, 2002, which is incorporated herein by reference and amphipathic asymmetric dendrimers, described by Shah, et al., Int. J. Pharm, 208:41-48, 2000, which is incorporated herein by reference. Poly propylene imine dendrimers, described in Tack, et al., J. Drug Target, 14:69-86, 2006, which is incorporated herein by reference; and other dendrimers described above, can be chemically modified to reduce toxicity, for example, as described in Tack, et al.

Dendrimers complex with nucleic acids as do other cationic polymers with high charge density. In general, the dendrimer-nucleic acid interaction is based on electrostatic interactions. Dendrimers can be conjugated with other molecules, such as cyclodextrins to increase efficiency of systemic delivery of dendrimer-nucleic acid complexes. (See Dufes, et al., Adv. Drug Del. Rev, 57, 2177-2202, 2005, and Svenson and Tomalia, Adv. Drug Del. Rev., 57, 2106-29, 2005, both of which are incorporated herein by reference.) Some dendrimers have a flexible open structure that can capture small molecules in their interior, and others have an inaccessible interior. (See Svenson and Tomalia, Adv. Drug Del. Rev., 57, 2106-29, 2005.)

In still further embodiments, oligonucleotides are complexed with additional polymers to aid in delivery (See e.g., U.S. Pat. Nos. 6,379,966, 6,339,067, 5,744,335; each of which is herein incorporated by reference. For example, polymers of N-2-hydroxypropyl methylacrylamide are described in U.S. patent publication number 2006/0014695, which is incorporated herein by reference. Similar cationic polymers are described in International Patent Publication number WO 03/066054 and U.S. patent publication number 2006/0051315, both of which are incorporated herein by reference. Other polymers are described by Intradigm Corp., Rockville, Md.).

5. Other Delivery Methods

In still further embodiments, the controlled high pressure delivery system developed by Mirus (Madison, Wis.) is utilized for delivery of oligonucleotides. The delivery system is described in U.S. Pat. No. 6,379,966, which is incorporated herein by reference.

B. Formulations, Administration and Uses

The compositions of the present invention may be administered orally, parenterally, by inhalation spray, topically, rectally, nasally, intraocularly, buccally, vaginally, or via an implanted reservoir. The term “parenteral” as used herein includes subcutaneous, intravenous, intramuscular, intra-articular, intra-synovial, intrasternal, intrathecal, intrahepatic, intraleRuysschaersional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, isotonic sodium chloride solution, and dextrose solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In embodiments where oligomers are prepared in liposomes, the oligomer/liposome formulations may lyophilized or spray-dried for storage. Suitable cryoprotectants and spray-drying protectants may include sugars, for example, but not limited to, glucose, sucrose, trehalose, isomaltose, somaltotriose, and lactose. Other cryoprotectants may include dimethylsulfoxide, sorbitol and other agents that alter the glass phase melting temperature (Tₘ). Preparations may include anti-adherents such as magnesium stearate and leucine, buffers, such as Tris or phosphate buffer, and chelating agents, such as EDTA.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In several embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration.

C. Biomarkers

Embodiments of the present invention may include detection of one or more protein biomarkers from a biological sample, after administration of oligonucleotides or pharmaceutical compositions of the present invention, to assess whether BCL2 expression is affected after administration of said oligonucleotides or pharmaceutical compositions of the present invention.

As used in the present application, a “biological sample” means any material or fluid (blood, lymph, etc.) derived from the body of a subject, that contains or may contain genomic DNA (chromosomal and mitochondrial DNA) or other oligonucleotides such as, for example, mRNA that derive from genomic DNA. Also included within the meaning of the term “biological sample” is an organ or tissue extract and culture fluid in which any cells or tissue preparation from a subject has been incubated. Methods of obtaining biological samples are well known in the art. Extraction of proteins from a biological sample may be performed using well-known methods in the art. In some embodiments, the level of protein biomarkers in a sample may be assayed directly (i.e., without a extraction step).

“Marker” in the context of the present invention refers to an organic biomolecule, particularly a polypeptide, which is differentially present in a sample taken from subjects after administration of oligonucleotides or pharmaceutical compositions of the present invention as compared to a comparable sample taken from the subject prior to that administration of oligonucleotides or pharmaceutical compositions of the present invention. For example, a marker can be a polypeptide (having a particular apparent molecular weight) which is present at an elevated or decreased level in samples of prostate cancer patients compared to samples of patients with a negative diagnosis.

“Organic biomolecule” refers to an organic molecule of biological origin, e.g., steroids, amino acids, nucleotides, sugars, polypeptides, polynucleotides, complex carbohydrates or lipids.

The phrase “differentially present” refers to differences in the quantity of a polypeptide (of a particular apparent molecular weight) present in a sample taken from patients having prostate cancer as compared to a comparable sample taken from patients who do not have prostate cancer (e.g., have benign prostate hyperplasia). A polypeptide is differentially present between the two samples if the amount of the polypeptide in one sample is significantly different from the amount of the polypeptide in the other sample.

For example, a polypeptide is differentially present between the two samples if it is present in an amount (e.g., concentration, mass, molar amount, etc.) at least about 150%, at least about 200%, at least about 500% or at least about 1000% greater than it is present in the other sample, or if it is detectable in one sample and not detectable in the other.

A “test amount” of a marker refers to an amount of a marker present in a sample being tested. A test amount can be either in absolute amount (e.g., ng/ml) or a relative amount (e.g., relative intensity of signals).

A “control amount” of a marker can be any amount or a range of amount which is to be compared against a test amount of a marker. For example, a control amount of a marker can be the amount of a marker in a subject prior to, or during administration of administration of oligonucleotides or pharmaceutical compositions of the present invention. A control amount can be either in absolute amount (e.g., ng/ml) or a relative amount (e.g., relative intensity of signals).

“Eluant” or “washing solution” refers to an agent that can be used to mediate adsorption of a marker to an adsorbent. Eluants and washing solutions also are referred to as “selectivity threshold modifiers. Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of a marker from all other markers in a mixture.

Rather, any separation that allows the distinction between at least two markers suffices.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g, by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochemical, biochemical, immunochemical, or chemical means. For example, useful labels include 32p, 35S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. The detectable moiety can be incorporated in or attached to a primer or probe either covalently, or through ionic, van der Waals or hydrogen bonds, e.g, incorporation of radioactive nucleotides, or biotinylated nucleotides that are recognized by streptavidin.

The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, or a nucleotide sequence, which is the binding partner for a complementary sequence, to which it can specifically hybridize.

The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., P. D. Fahrlander and A. Klausner, BiolTechnology 6: 1165 (1988). Quantitation of the signal is achieved by, e.g., scintillation counting, densitometry, or flow cytometry.

“Measure” in all of its grammatical forms, refers to detecting, quantifying or qualifying the amount (including molar amount), concentration or mass of a physical entity or chemical composition either in absolute terms in the case of quantifying, or in terms relative to a comparable physical entity or chemical composition.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

A. Sample Sources For Markers

The sample is preferably a biological fluid sample Examples of biological fluid samples useful in this invention include blood, serum, urine, prostatic fluid, seminal fluid, semen, seminal plasma and prostate tissue (e.g., epithelial tissue, including extracts thereof).

B. Detection of Markers

After a sample is obtained, any suitable method can be used to detect the marker in a sample from a subject being tested. For example, gas phase ion spectrometry or an immunoassay can be used.

### 1. Mass Spectrometry

In one embodiment, the markers of this invention are detected using mass spectrometry, more preferably using gas phase ion spectrometry and, still more preferably, using surface-enhanced laser desorption/ionization mass spectrometry (“SELDI”). SELDI is an improved method of gas phase ion spectrometry for biomolecules. In SELDI, the surface on which the analyte is applied plays an active role in the analyte capture, desorption and/or desorption.

One popular method of gas phase ion spectrometry for biomolecules is MALDI (matrix-assisted laser desorption/ionization) mass spectrometry. In MALDI, the analyte is typically mixed with a matrix material that, upon drying, forms crystals that capture the analyte. The matrix material absorbs energy from the energy source which otherwise would fragment the bimolecular analytes.

### a) Preparation of Sample

(i) Pre-fractionation

In one embodiment, the sample can be pre-fractionated before being subjected to gas phase ion spectrometry. Pre-fractionation has the advantage of providing a less complex sample for analysis. On the other hand, it introduces an extra step in the analytic process that could be unattractive in, for example, a clinical setting. Samples can be pre-fractionated by any means known in the art, including, without limitation, size fractionation and chromatographic fractionation.

In one embodiment, samples can be pre-fractionated before analysis by gas-phase ion spectrometry. A preferred method of fraction includes a first fractionation by gel exclusion chromatography. Sizing columns which exclude molecules whose molecular mass is greater than 30 kDa are particularly useful for this.

Fractions of various sizes then can be examined directly or subjected to a second fractionation step based on anion exchange chromatography. Using an anion exchange Q spin column, markers can be eluted using a low strength buffer (e.g., about 10 mM to 50 mM Tris, HEPES or PBS) with salt at low to medium concentration (e.g., about 0.1 M to 0.6 M) and a non-ionic detergent at low concentration (e.g., TritonX 100 at about 0.05 to 0.2%). A particularly useful buffer is 20 mM Tris, 0.5 M NaCl and 0.1% TritonX 100. In another embodiment, the markers are eluted using a pH gradient.

(ii) Retentate Chromatography

In another embodiment, the sample is fractionated on a bio-chromatographic chip by retentate chromatography before gas phase ion spectrometry. A preferred chip is the Protein Chip® array available from Ciphergen Biosystems, Inc. (Palo Alto, Calif.). As described above, the chip or probe is adapted for use in a mass spectrometer. The chip comprises an adsorbent attached to its surface. This adsorbent can function, in certain applications, as an in situ chromatography resin. In basic operation, the sample is applied to the adsorbent in an eluent solution. Molecules for which the adsorbent has affinity under the wash condition bind to the adsorbent.

Molecules that do not bind to the adsorbent are removed with the wash. The adsorbent can be further washed under various levels of stringency so that analytes are retained or eluted to an appropriate level for analysis. Then, an energy absorbing molecule can be added to the adsorbent spot to further facilitate desorption and ionization. The analyte is detected by desorption from the adsorbent, ionization and direct detection by a detector. Thus, retentate chromatography differs from traditional chromatography in that the analyte retained by the affinity material is detected, whereas in traditional chromatography, material that is eluted from the affinity material is detected.

A useful adsorbent for the markers of the Marker Set is a metal chelate adsorbent and, in particular, copper. This surface also is usefully washed with a pH neutral buffer such as PBS. A preferred surface is IMAC3 from Ciphergen Biosystems, Inc. IMAC3 comprises a copper chelate adsorbent.

Another useful adsorbent for any of the markers of this invention is an antibody that specifically binds the marker. Chips comprising antibodies that bind to one or more markers are particularly useful for removing non-markers which do not bind to the antibodies and which function as “noise” in the detection process.

As will be evident to anyone skilled in the art, different markers may be more easily resolved using different combinations of adsorbents and eluants. (iii) Mixing the sample with an energy absorbing matrix

In MALDI applications the sample to be analyzed is mixed with an energy absorbing matrix prior to ionization and mass analysis. The sample/matrix mixture is then applied to the surface of an inert mass spectrometer probe. Suitable matrix materials are well know to those of skill in the art and include 3-hydroxypicolinic acid (3-hydroxy-2-pyridinecarboxylic acid), nicotinic acid, N-oxide, 2′-6′-dihydroxyacetophenone, gentisic acid (2,5-dihydroxybenzoic acid), a-cyano-4-hydroxycinnamic acid, ferulic acid (4-hydroxy-3-methoxycinnamic acid) and sinapinic acid (3,5-dimethoxy-4-hydroxycinnamic acid).

The resolving power of MALDI is limited by the complexity of the sample being analyzed. Therefore prefractionation or otherwise purifying the sample prior to MALDI analysis is preferred.

(iii) Modification of Marker Before Analysis

In another embodiment, the markers are modified before detection in order to alter their molecular weight. These methods may decrease ambiguity of detection. For example, the markers may be subject to proteolytic digestion before analysis. Any protease can be used. Proteases such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another embodiment, the markers can be modified by the attachment of a tag of particular molecular weight that bind specifically to molecular markers, further distinguishing them. b) Performance of laser desorption/ionization mass spectrometry After the marker is detected by mass spectrometry, preferably gas phase ion spectrometry, a test amount of marker can be determined. For example, a signal is displayed at the molecular weight of the marker of interest. Based on the strength or magnitude of the displayed signal, the amount of marker in a sample being tested can be determined. It is noted that the test amount of marker in a sample need not be measured in absolute units, but can be in relative units as long as it can be compared qualitatively or quantitatively to a control amount of a marker. For example, the amount of the marker detected can be displayed in terms of relative intensity based on the background noise. Preferably, the test amount and the control amount of markers are measured under the same conditions.

If desired, the absolute amount of a marker can be determined by calibration. For example, a purified, known marker can be added in increasing amounts to different spots of adsorbents on the probe surface. Then peaks from each spot can be obtained and plotted in a graph against the concentration of known marker protein at each spot. From the peak intensity vs. concentration plot, the absolute amount of a marker in any sample being tested can be determined

### 2. Immunoassay Detection

In another embodiment of the detection method, an immunoassay can be used to qualitatively or quantitatively detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 2nd Ed. (Sambrook et al., Cold Spring Harbor Laboratory, NY 1989).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al, Science 246:1275-1281 (1989); Ward et al., Nature 341:544-546 (1989)).

After the antibody is provided, a marker can be detected and/or quantified using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Ten, eds., 7th ed. 1991).

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. (See e.g. Xiao et al, Cancer Research 62: 6029-6033 (2001)) The sample is preferably a biological fluid sample taken from a subject. Examples of biological fluid samples include blood, serum, urine, prostatic fluid, seminal fluid, semen, seminal plasma and prostate tissue (e.g., epithelial tissue, including extracts thereof). In a preferred embodiment, the biological fluid comprises seminal plasma. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay techniques are well-known in the art, and a general overview of the applicable technology can be found in Harlow & Lane, supra. The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. As noted above, the test amount of marker need not be measured in absolute units, as long as the unit of measurement can be compared to a control amount.

### C. Biomarkers

Suitable biomarkers may include, for example, one more of the following proteins: phosphpLeptin, GM-CSF, IL-20, MIP-1a (CCL3), MMP-7, SAA and sCD40L.

In some aspects, suitable biomarkers include phosphorylated BCL2, active capsase-3, PARP, leptin, IL-1RA, IL-17a, MCP-1, MIP-1f3, and IP10 or combinations thereof.

In some aspects, suitable biomarkers include lymphocyte counts and platelet counts. Methods of obtaining and counting platelets and lymphocytes from blood plasma are well-known in the art.

Other biomarkers that correlate with the expression of BCL2 in vivo may be used in this invention. Suitable biomarkers may be other genes implicated in the pro-. or anti-apoptotic pathways, or mixtures of both.

### D. Kits

Embodiments of the present invention, include kits for determining the down-regulation of the expression of BCL2 after administration of a test compound for the treatment of a BCL2 mediated cancer in a subject having cancer comprising: probes for detecting the levels of one or more of a biomarker in the biological sample, wherein the biomarker is selected from the group consisting of: phosphorylated BCL2, active capsase-3, PARP, lymphocyte counts, platelet counts, leptin, IL-1ra, IL-17a, MCP-1, MIP-1β, and IP10, or combinations thereof.

Suitable probes may include any other probes cited herein. Christine F. Garcia and Steven H. Swerdlow (2009) Best Practices in Contemporary Diagnostic Immunohistochemistry: Panel Approach to Hematolymphoid Proliferations. Archives of Pathology & Laboratory Medicine: May 2009, Vol. 133, No. 5, pp. 756-765 https://www.labcorp.com/wps/wcm/connect/IntOncologyLib/integratedoncology/resources/p dfs/test+requisition+forms/test-requisition-form-hematology-oncology

### IV. Examples of Cancer Therapies

The following examples are provided in order to demonstrate and further illustrate certain preferred embodiments and aspects of the present invention and are not to be construed as limiting the scope thereof.

### Example 1

### Efficacy of PNT-2258 by Cancer Type

In previous animal model studies on the effectiveness of PNT-2258 alone and in combination with other chemotherapeutic agents, the efficacy of PNT2258 appeared to increase with increasing BCL2 expression in a particular cancer (see FIG. 1; i.e. Daudi-Burkitts lymphoma; prostate (PC-3); melanoma (A375); diffuse large cell lymphoma (WSU-DLCL2)).

### Example 2

### Experimental Design of Dose Range Study in Human Patients with Various Cancers

An open-label, single-arm, Phase 1 dose-escalation study of PNT2258 in human patients with advanced solid tumors Patients received PNT2258 as an intravenous infusion over 2 hours once daily for 5 consecutive days (Days 1-5) of a 21-day cycle (3 weeks). The initial dose level was 1 mg/m². The dose was doubled until the 64 mg/m² dose level is completed (e.g., Cohort 1=1 mg/m²; Cohort 2=2 mg/m²; Cohort 3=4 mg/m²). Thereafter, dose escalation should proceed with increases of 30 mg/m² increments with the next dose level at 90 mg/m² and continuing to 120 mg/m² and 150 mg/m² in subsequent dose escalations. If a patient dosed at <64 mg/m² experienced a >Grade 2 toxicity during Cycle 1 (excluding alopecia, nausea or vomiting with less than maximal antiemetic treatment, and diarrhea with less than maximal antidiarrheal treatment), then doses were increased in increments of 33% using cohorts of 3-6 patients guided by the observance of DLTs (dose-limiting toxicities).

DLT on this study were defined as the following treatment-related events experienced during Cycle 1:
Grade 4 neutropenia of greater than 5 days duration, or Grade 3 or greater febrile neutropenia of any duration.
Grade 4 thrombocytopenia.
Any Grade 3 or greater non-hematologic toxicity (except alopecia, nausea/vomiting well-controlled with antiemetics, and laboratory abnormalities felt to be clinically insignificant or that were elevated at baseline).
Any toxicity resulting in a treatment delay beyond 2 weeks.
Acute infusion reaction that requires removal from the study (i.e., does not resolve to baseline or ≦Grade 1 after infusion interruption and resumption at a slower rate).
A 2-Grade increase in AST(SGOT)/ALT(SGPT) for patients with baseline Grade 1 or 2 abnormalities.

The dose at the beginning of each cycle was calculated based on the patient's computed body surface area obtained prior to dosing on Cycle 1 Day 1 unless there was >10% change since baseline. If there was a >10% change, the current weight was used to calculate the dose for that cycle.

If the patient developed an acute reaction to treatment during infusion, the infusion rate may be reduced according to the investigator's judgment or the infusion may be interrupted until the reaction resolves to baseline or <Grade 1; however, total infusion time, including interruptions, may not exceed 6 hours. If toxicities did not resolved to baseline or <Grade 1, the infusion was terminated and the patient was removed from the study. Patients experiencing clinically significant infusion reactions received premedication prior to subsequent dosing.

The majority of the patients received PNT2258 as an intravenous infusion over 2 hours once daily for 5 consecutive days (Days 1-5) of a 21-day cycle (3 weeks). However, several patients received PNT2258 at a third (six hours) or half (4 hours) the dose rate either during Cycle 1 or Cycle 2. Further, several patients received PNT2258 for 4 consecutive days rather than 5 consecutive days or several patients received PNT2258 as part of a 28-day cycle (4 weeks). Overall, the dose range of 1-150 mg/m² was well-tolerated. Dose rate and dose schedule were adjusted to patient tolerability and availability to return to the clinic for dosing, thereby providing support for PNT2258 at different dose regimens.

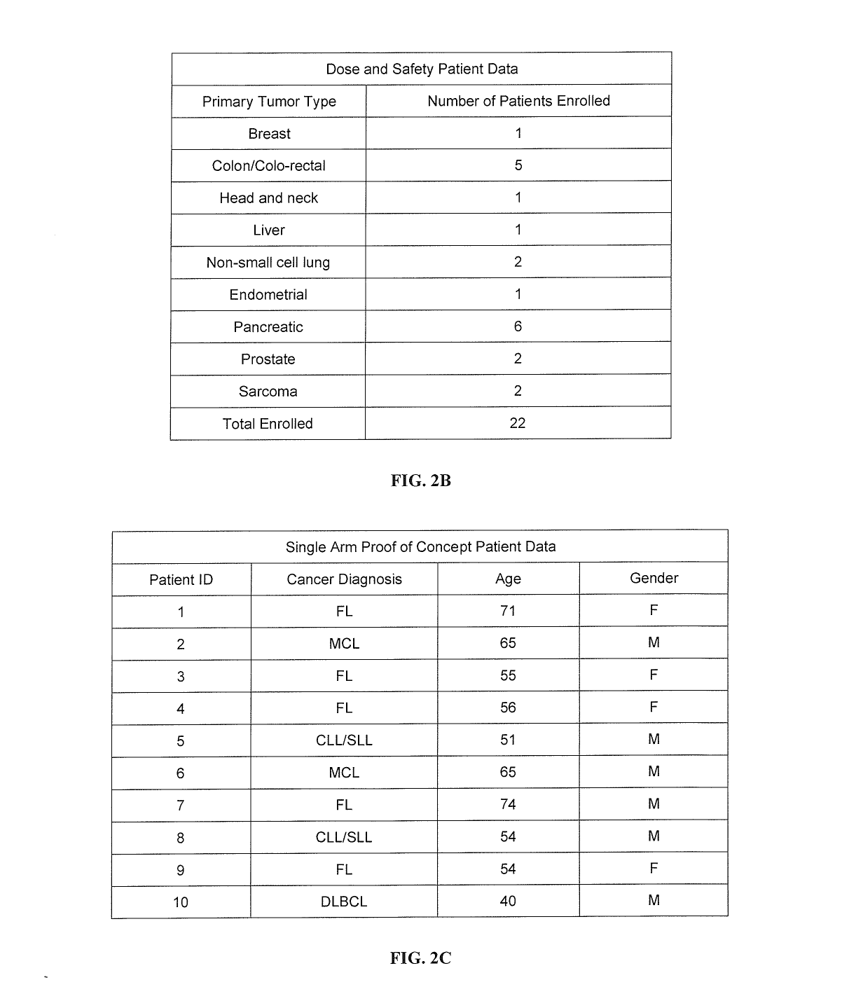

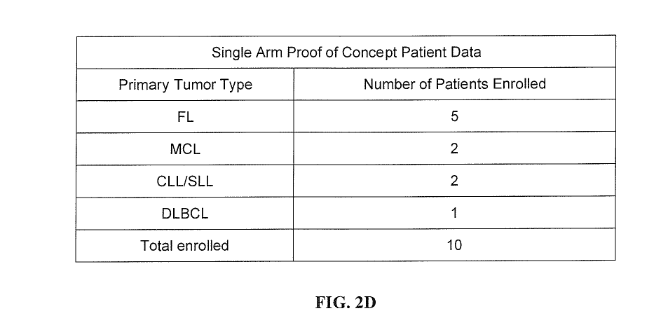

FIG. 2 provides the patient information and assignment into the dose and safety study, and also shows the number of patients having a particular cancer type by study.

### Example 3

### Tumor Response During Study

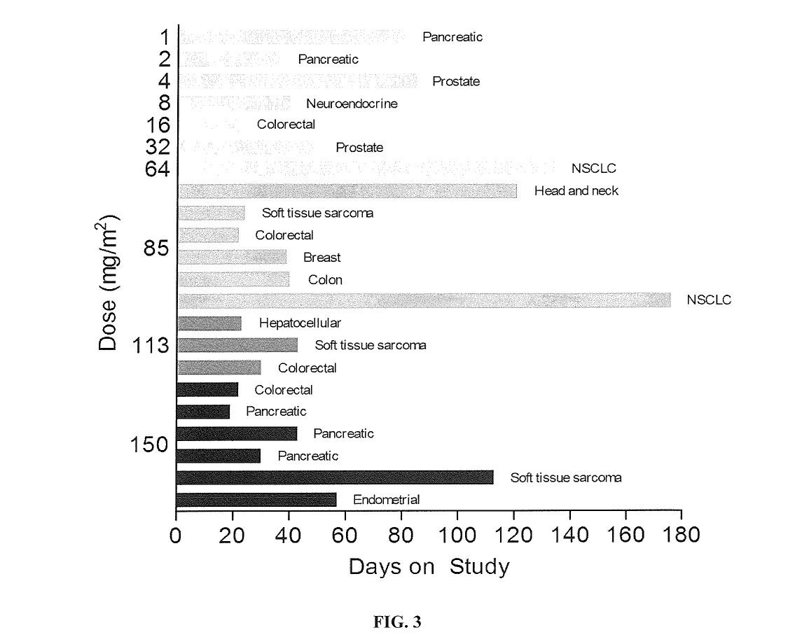

The median number of cycles the subject patients remained in the study is two cycles. The median time a patient remained in the study is 6 weeks. Note that several patients treated with PNT2258 remained in the study for 6-8 cycles (i.e., 16-24 weeks), as shown in FIG. 3. It is interesting to note that the patients who stayed on study longest due to stable disease correspond well with tumor types known to be BCL2-dependent and are in tissues of the reticuloendothelial system (RES).

An additional factor that emerged from the plasma biomarker results, (see below), is the upregulation of leptin seen in most patients. Not to be limited by theory, many of the best-responding patients had glucose and cholesterol profiles that are suggestive of metabolic syndrome or an increased inflammatory state. It is well known that low-grade chronic inflammatory states induce up-regulation of many pathways that enhance cellular survival. These inflammatory states have a strong association with many types of cancer. BCL2 is one of the pathways that is up-regulated. Leptin signaling is another pathway that is associated with cancer, in particular breast cancer.

Patients having persistent low-grade inflammation have been shown to be resistant to both insulin and leptin signaling. Therefore, although leptin up-regulation is observed which may confer resistance to BCL2 down-regulation these patients do not appear to respond to the compensatory marker to the same degree as those patients having a somewhat lower degree of systemic inflammatory marker traffic due to their diminished leptin signaling receptivity. Alternatively, since increased leptin is associated with increased BCL2 transcription (see Lam, Q. L. K. et al. (2010) Proc. Nat'l. Acad. Sci. USA 107:13812-13817), the feedback loop created by leptin may create an environment that PNT2258's mode of action of blocking BCL2 transcription is enhanced. PNT2258 results in an IL-RA (a marker correlated with an anti-inflammatory effect) increase, further supporting the beneficial and anti-inflammatory effect that may be associated with patients with concomitant inflammation (e.g. those with metabolic syndrome).

### Example 4

### Analysis of BCL2 Expression in Subject Peripheral Blood Mononuclear Cells (PBMCs) Pre- and Post-Dose of PNT2258

Peripheral blood mononuclear cells (PBMCs) are widely used as surrogates of tumor tissue/cells if the protein of interest is expressed in both the tumor cells and the PBMCs.

PBMC specimens from patients were collected at the START clinic in San Antonio, Tex. as part of the Phase I study with PNT2258. The specimens were delivered to the test site on dry ice and stored frozen at −70° C. until processing. The PBMCs, which were frozen at the clinical site as suspensions in 0.5 mL PBS, were thawed on ice in the presence of concentrated 10x lysis buffer (final concentration 0.1% Triton X-100, 20 mM EDTA, 5 mM Tris pH 8, 1 mM sodium orthovanadate, 2 mM PMSF, and 1% each protease and phosphatase inhibitor cocktails), then sonicated in a water bath for 10 minutes and frozen at −70° C. for 24 hours. The lysates were clarified by centrifugation at 7,000 RCF for 15 minutes at 4° C. and the supernatants were removed to fresh tubes. Twenty microliter aliquots were withdrawn for measurements of protein concentration using micro BCA assay (Thermo Fisher Scientific, Rockford, Ill.) and the remaining samples were frozen at −70° C. until assayed for BCL2, phosphorylated BCL2, GAPDH, active caspase-3 and active PARP.

### ELISA Assays

The following human-specific quantitative ELISA kits were used: Platinum ELISA for BCL2 (P.N. BMS244/3, eBioscience, Vienna, Austria), Quantikine human active caspase-3 ELISA (P.N. KM300, R&D Systems, Minneapolis, Minn.), active (cleaved) PARP (214/215) (P.N. KH00741, Invitrogen, Camarillo, Calif.), and human glyceraldehyde 3-phosphate dehydrogenase (GAPDH; P.N. KT-16442, Kamiya Biomedical Company, Seattle, Wash.). Protease inhibitor cocktail (P.N. P8340) and broad range phosphatase inhibitor cocktails (P.Nos. P5726 and P2850, respectively) and carbonate-bicarbonate buffer capsules (P.N. C3041-50CAP) were obtained from Sigma-Aldrich, St. Louis, Mo.

For development of phospho-BCL2 (Ser70) ELISA, Rabbit IgG anti-human phospho-BCL2 (Ser70) polyclonal antibody (Pierce/Thermo Fisher Scientific Inc, Rockford, Ill.; P.N. PA1-14063) and rabbit polyclonal antibody to human BCL2 (P.N. ab59348, Abcam, Cambridge, Mass.) were used. A synthetic peptide (R-T-phospho-S-P-L; further referred to as a ‘pentapeptide’; 96.89% purity) corresponding to the amino acid sequence around the phosphorylation site of human phospho (ser70) BCL2, also used as an immunogen in preparation of the antibody to phospho-BCL2 (Ser70) (PA1-14063), was purchased from Biomatik, Cambridge, ON, Canada.

Paclitaxel-stimulated Jurkat cell lysate (EMD Millipore Corporation, St. Charles, Mo.; P.N. 47-206) was used as a positive control and unstimulated Jurkat cell lysate (EMD Millipore Corporation, St. Charles, Mo.; P.N. 47-206) was used as a negative control in development of phospho-BCL2 (Ser70) ELISA. Cliniplate EBV 96-well (P.N. 95019330) high affinity protein binding plates, QuantaBlu fluorogenic peroxidase substrate kit (P.N. 151569), Restore Western Blot Stripping Buffer (P.N. 21059) and SuperSignal West Pico Stable Peroxide and Luminal Enhancer solutions (P.Nos. 1859674 and 1859675) were purchased from Thermo Fisher Scientific (Waltham, Mass.).

### Western Blots

Five pit aliquots of PBMC lysates were added to 15 μL of reducing SDS buffer and resolved in 4-15% Criterion TGX SDS polyacrylamide gels (Bio-Rad Laboratories, Hercules, Calif.) with biotinylated protein ladder (Cell Signaling Technology, Beverly, Mass.) and Kaleidoscope pre-stained protein standard (Bio-Rad Laboratories, Hercules, Calif.). The gels were transblotted to Hybond-C nitrocellulose (Amersham Biosciences, Piscataway, N.J.) and the membranes were blocked in 5% ECL Advance blocking solution (Amersham Biosciences, Piscataway, N.J.). The membranes were incubated overnight at 4° C. with the following primary rabbit anti-human monoclonal antibodies: phospho-BCL2 (Ser70) (clone 5H2, P.N. 2827, Cell Signaling, Danvers, Mass.) and total BCL2 (P.N. ab59348, Abcam, Cambridge, Mass.), both at 1:1,000 dilution. Phospho-BCL2 probing was done first. Then for the detection of total BCL2 the membranes were first stripped by 15 min incubation with the stripping buffer, washed and blocked as described above. Anti-rabbit IgG HRP conjugate was used as secondary antibody (P.N. SA1-5910, Thermo Fisher Scientific, Rockford, Ill.) at 1:2,000 dilution and 1 hour incubation at room temperature. All antibodies were diluted with 2.5% ECL Advance blocking solution. Target proteins were visualized by enhanced chemiluminescence with ECL Advance Western Blot detection kit and captured on Hyperfilm-ECL film (both from Amersham Biosciences). Molecular masses of target proteins were verified against standards. Images were obtained with a densitometer and quantitfied using ImageQuant software (Molecular Dynamics).

### Immunodetection of Proteins by ELISA

ELISAs for total BCL2, active caspase-3, active PARP and GAPDH were performed according to the supplier's instructions with appropriate standards included in these kits. All standards were measured in duplicate in seven serial dilutions; lysis buffer will be used as a negative control. Although it was originally proposed to test samples at a total protein at 0.1 mg/mL, because of low total protein concentration in many lysates, the samples were assayed in triplicate without additional dilutions.

### Development of ELISA to Phosphorylated BCL2 (Ser70)

To develop an in-house ELISA protocol for measuring levels of human phospho BCL2 (Ser70), three different sets of conditions based on protocols for ELISA development 3; 4 were examined in a 96-well format using the following setup in rows A-H, columns 1-12:

| | | | | |
|---|---|---|---|---|
| | A | pentapeptide | 20,000 | ng/mL |
| | B | pentapeptide | 2000 | ng/mL |
| | C | pentapeptide | 200 | ng/mL |
| | D | pentapeptide | 20 | ng/mL |
| | E | pentapeptide | 2 | ng/mL |
| Pos. Ctrl | F | Jurkat T-cells paclitaxel stimulated | 5 | μg/mL |
| Neg. Ctrl | G | Jurkat T-cells 7.6.4 | 10 | μg/mL |

| | | | |
|---|---|---|---|
| Blank | H | Antigen dilution buffer | 0 |

### Direct Capture ELISA (in PBS)

Bovine serum albumin (10 μg/mL) in PBS was used as the antigen dilution buffer. The pentapeptide in 5 serial dilutions (rows A-E), paclitaxel-stimulated Jurkat cell lysate (row F), unstimulated Jurkat cell lysate (row G), and the antigen dilution buffer alone (row H) were plated in a volume of 50 μL in columns 1-12 of the 96-well Cliniplate. The plate was sealed and stored at 4° C. overnight. The wells were washed 4 times (1 min each) with shaking on a horizontal shaker at 100 rpm) with 340 μL of a wash buffer (0.05% Tweeen-20 in PBS) and blocked in 100 μL 5% non-fat dry milk in PBS for 2 hours at room temperature. The wells were washed as above and incubated for 2 hours at room temperature with the antibody to phospho BCL2 (Ser70) (ab28819) at 1:10,000 prepared in an antibody dilution buffer (1% non-fat dry milk in PBS). This was a recommended dilution of the antibody for ELISA per supplier's protocol. After 4 washes as above, rows A-D were incubated at room temperature for 1 hour with goat-anti-rabbit HRP conjugate diluted 1:2000 in the antibody dilution buffer, while rows E-H were incubated with the same antibody at 1:5000 dilution. Following washes as above, 100 μL QuantaBlu fluorogenic peroxidase substrate solution was added to each well and incubated at room temperature for 30 min, then 90 μL aliquots were transferred to corresponding wells of a clear bottom white fluorescence plate and the fluorescence (excitation at 355 nm. emission at 460 nm) was recorded.

### Direct Capture ELISA (Carbonate/Bicarbonate Buffer pH 9.6)

The protocol was a replica of that described above except 0.2 M carbonate/bicarbonate buffer pH 9.6 was used to prepare antigens for plating in order to maximize protein binding to the plate. Half of the plate (columns 1-6) was blocked in 5% non-fat milk in PBS and the second half (columns 7-12) was blocked in 5% BSA in PBS. Subsequent antibody solutions were prepared either in 1% milk or BSA to match the composition of the blocking solution. In addition, the first antibody was examined in multiple dilutions at 1:2000 (columns 1, 4, 7, and 10), 1:4000 (columns 2, 5, 8, and 11) and 1:8000 (columns 3, 6, 9, and 12). The second antibody was tested at 1:1000 (columns 1-3 and 7-9) and 1:2000 (columns 4-6 and 10-12).

### Sandwich ELISA

All 96 wells of a Cliniplate were coated with 100 μL solution of phospho BCL2 (Ser70) antibody (capture antibody) diluted 1:1000 in 0.2 M carbonate/bicarbonate buffer pH 9.6, and the plate was sealed and incubated at 4 C overnight. Following washes as above, antigen solutions prepared in 0.2 M carbonate/bicarbonate buffer pH 9.6 were plated in rows A-H, columns 1-12, the plate was sealed and incubated at 4 C overnight. The next steps were performed following an outline for direct capture ELISA in carbonate/bicarbonate buffer pH 9.6 except a rabbit polyclonal antibody to BCL2 was used as the first antibody.

### Results

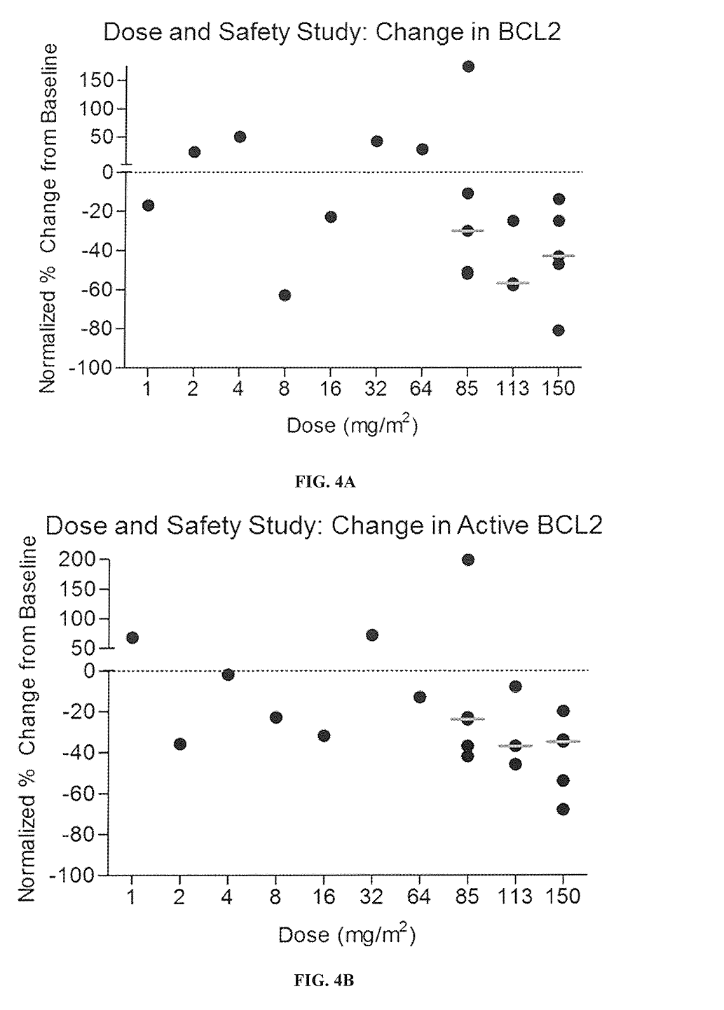

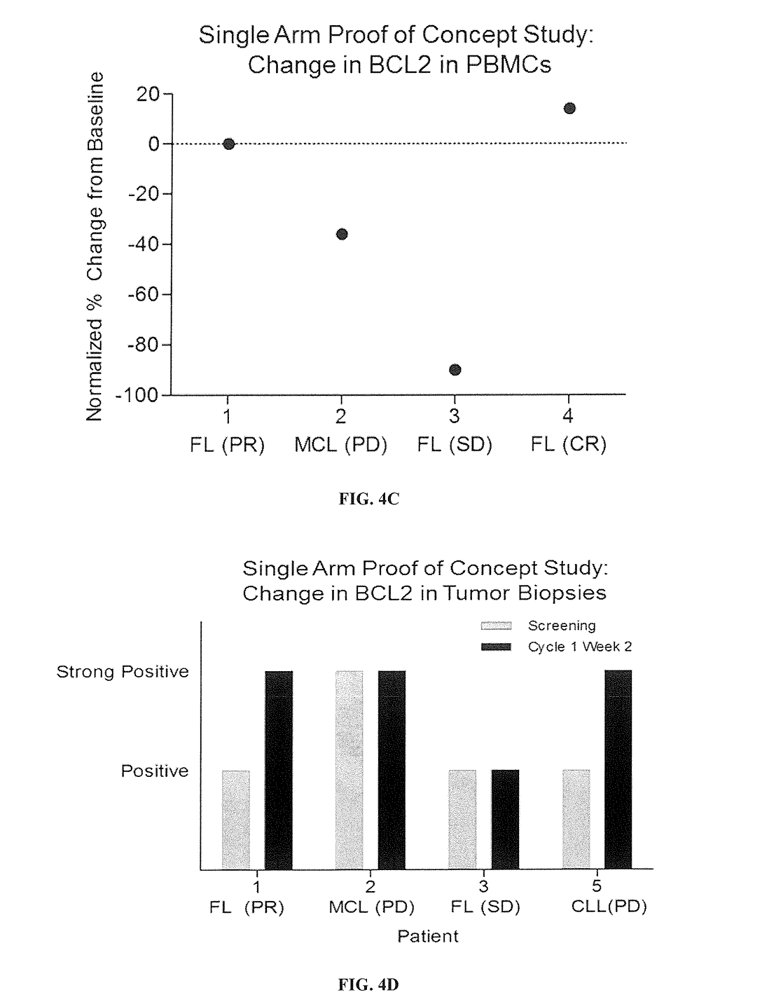

The percent change in BCL2, activated (phosphorylated) BCL2, caspase-3 and PARP cleavage from baseline (pre-dose) and post-Day 5 dosing with PNT2258 are shown in FIG. 4 (left). The majority of patients demonstrated a reduction in BCL2 following PNT2258 dosing. Further evidence is provided for a reduction of BCL2 in the observed increase in capsase-3 and PARP cleavage. A reduction in BCL2 initiates a cascade of events leading to the activation of caspase enzymes and the cleavage of PARP, which are hallmarks of apoptotic cell death.

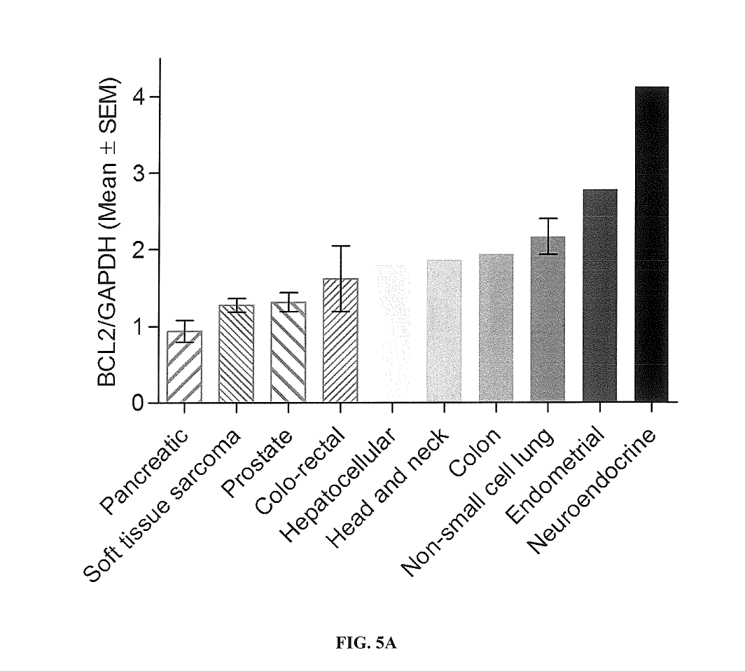

A dose-dependent decrease in BCL2 was noted following PNT2258 treatment with a dose-saturation at approximately 100 mg/m². (FIG. 4, right). Examining the data across subject patient tumor type yields interesting results, where there appears to be differences in the degree of BCL2 reduction with pancreatic, lung and sarcoma cancers showing the largest percentages. (FIG. 5). Of note, prostate and colorectal cancers appear to respond to PNT2258 by increasing BCL2, perhaps in response to treatment.

The extent of BCL2 knockdown in PBMCs is likely an underestimation of the ability of PNT2258 to modulate BCL2 levels. This is due to the fact that PBMCs consist of NK and T cells (lymphocytes, basophils, monocytes, eosinophils) and that this measurement is highly time-dependent. Reductions in lymphocytes, basophils, monocytes are noted following PNT2258 treatment. Therefore, the PBMC population being sampled may be (1) cells that are quiescent and not actively cell cycling or (2) newly released cells. It is further complicated by fact that in cells are likely cleared when BCL2 levels are highly suppressed.

### Example 5

### Analysis of Lymphocytes and Platelet Number/Counts in Patients Dosed with PNT2258

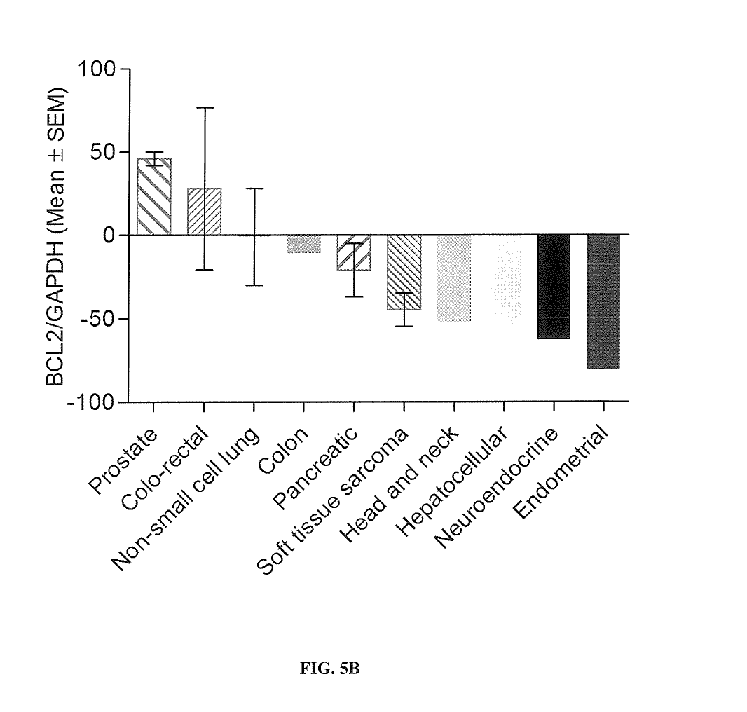

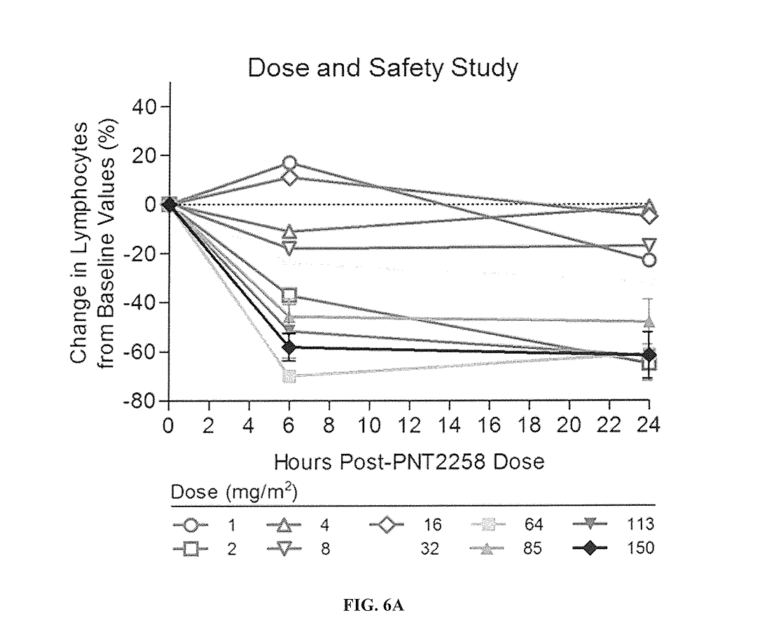

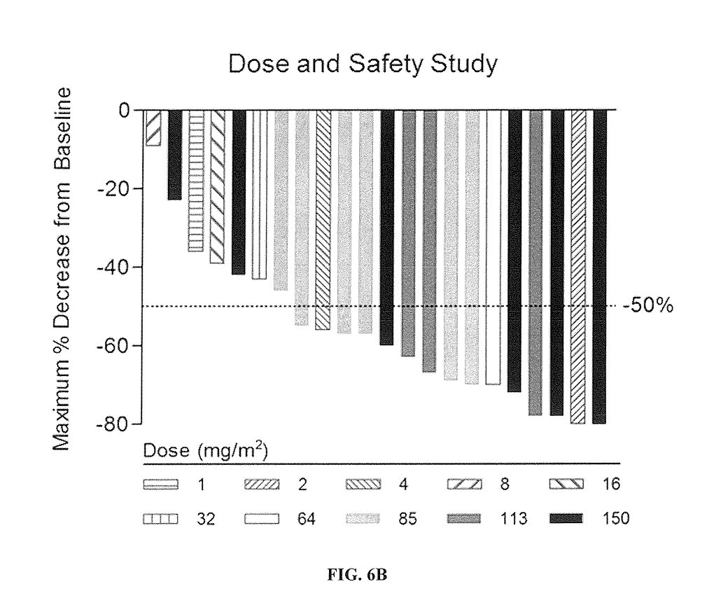

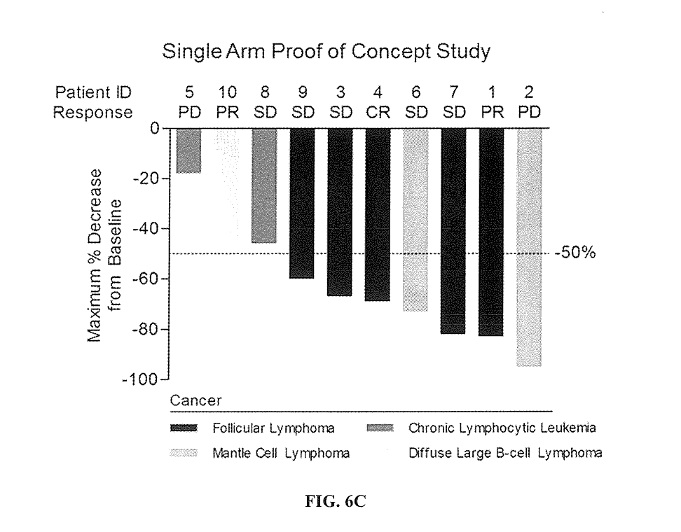

Lymphocytes are intense expressers of BCL2, and their clearance is BCL2 dependent. BCL2 sequesters Bim, a pro-apototic protein belonging to a distinct subgroup of proteins resembling other BCL2 family members within the short BH3 domain. Bim is essential for hemopoietic cell homeostasis. PNT2258 caused a transient, but clearly measurable decrease in lymphocytes due to targeting of BCL2. (FIG. 6). Lymphocytes appear to decrease during PNT2258 administration, with dose saturation around 100× administration.

Thrombocytopenia is a common side effect of chemotherapeutic agents. For BCL2-targeted agents, platelet reductions can represent a dose-limiting toxicity. This toxicity may result from an on-target effect of modulating BCL2 family members thereby causing enhanced apoptotic clearance of platelets.

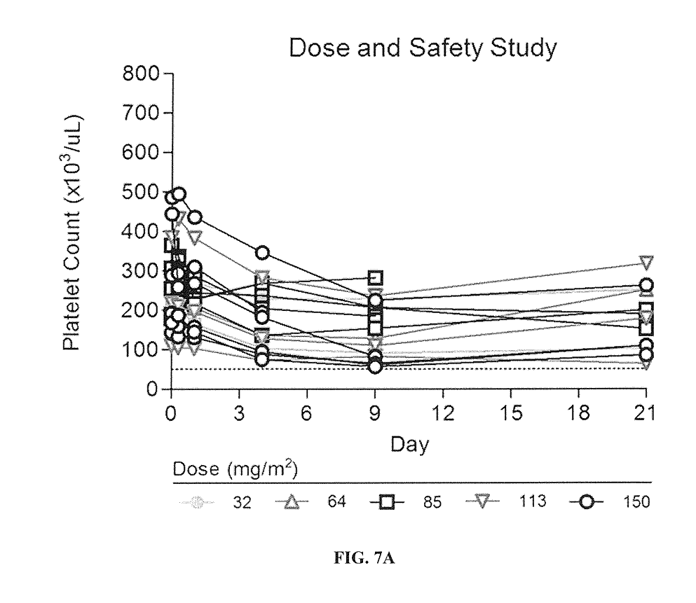

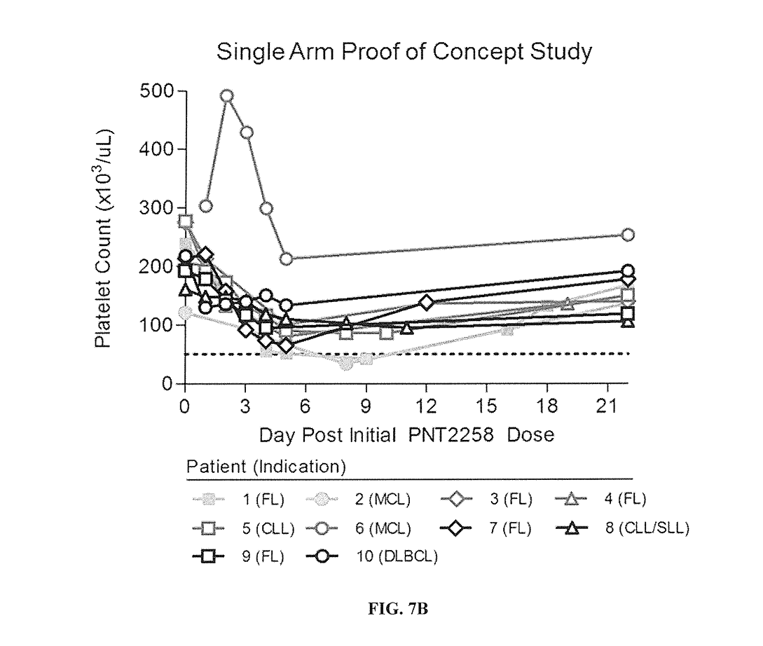

The thrombocytopenia observed with PNT2258 may be a function of BCL2 suppression and a liposome carrier effect on bone marrow and spleen (RES tissues), rather than on circulating platelets. The dose-dependent platelet nadir occurs at days 5-9, suggesting effects that are primarily due to megakaryocytes and on-target bcl-2 effect. The data suggests a downward trend in platelet counts following PNT2258 dosing that began at Cohort 7 with effects observed on Day 5 and nadir on Day 9. (FIG. 7) The timing of the decrease and the transient effect seen in this study is consistent with the idea that PNT2258 influences megakaryotes rather than circulating platelets. Platelets are anuclear and thus should not be influenced by PNT2258. On the other hand, megakaryocytes shed platelets following their maturation. Megakaryocytes are produced primarily by the bone marrow and spleen and tailor their cytoplasm and membranes to enable platelet biogenesis through an enlargement and endomitosis, a process that amplifies DNA by as much as 64-fold. Not to be limited by theory, it is at this point PNT2258 is believed to act, and therefore may influence platelet production and account for the transient and delayed downward trend of platelets noted at higher doses. In contrast, an immediate thrombocytopenia is observed with ABT-263, likely due to its targeted disruption of BCL2, Bcl-xL and Mcl-1 in circulating cells, causing their clearance.

### Example 6

### Plasma Protein Biomarkers Post-PNT2258 Administration in Mouse Models

A multiplex immunoassay was performed in two mice models post-administration with PNT-2258, for use as a comparison with an immunoassay performed on the human subject enrolled in the above study (data provided in next Example).

Immunocompetent female Balb/c mice 16-18 weeks old weighing approximately 25 g were purchased from Taconic Farms (Hudson, N.Y.). Animals were allowed to acclimatize to laboratory surroundings for at least 72 hours after delivery. The following preparations were used for injections: PNT2258, PNTE (empty liposomes), and scrambled oligonucleotide encapsulated in the same liposome composition as PNT2258 (scrambled) were used were diluted with sterile PBS to a final concentration of 2 mg/mL. Mice were injected with 120 uL preparations corresponding to the dose of 10 mg/kg. The animals were sacrificed 24 hours post-injection and immediately exsanguinated for the preparation of plasma.

Similar protocols were used for Female C.B-17 SCID mice between 4-6 weeks old were implanted with WSU-DLCL2 xenograft fragments. These mice were treated with PNT2258 or scrambled control when tumors achieved volumes of 300-400 mm³.

PNT2258 (PNT100 encapsulated in SMARTICLES) versus a scrambled sequence of PNT100 encapsulated in SMARTICLES were administered in normal mice (having adaptive and innate arms of immunity) or mice with WSU-DLCL2 tumor xenografts (having only innate immunity).

### Methods for Luminex Multiplex Immunoassays

The assays followed standard protocols as described for murine analytes (Streeper R. T., Diaz A., Campos D., Michalek J., Louden C, Furmaga W., Izbicka E. (2011) Syntra-5 downregulates inflammatory signaling in obese type 2 diabetes murine model in vivo. Curr. Topics Nutraceutical Res. 9:1-12) and human analytes (Izbicka E., Streeper R. T., Michalek J., Louden C., Diaz A., Campos D. (2012) Plasma biomarkers distinguish non-small cell lung cancer from asthma and differ in men and women. Cancer Genomics Proteomics 9(1):27-35). Methods for the Luminex bioassays are discussed further in e.g., U.S. Pat. No. 7,888,051, and Izbicka, E. et al. (2012) Cancer Genomics & Proteomics 9:27-36.

The following plasma protein analytes were assayed in mice using Procarta kits from Affymetrix (Fremont, Calif., USA).

Mouse 37-plex

| | | |
|---|---|---|
| Adiponectin | IL-3 | IL-17A/CTLA-8 |
| MIP-1 alpha/CCL3 | BTC/Betacellulin | IL-4 |
| IL-21 | MIP-2/GRO beta/CINC3 | Eotaxin/CCL11 |
| IL-5 | IL-23 p19 | RANTES/CCL5 |
| G-CSF/CSF-3 | IL-6 | IP-10/CXCL10 |
| RANKL/TNGSF11 | GM-CSF/CSF-2 | IL-9 |
| Leptin/LEP | TGF-beta 1 | Gro alpha/KC/CINC1 |
| IL-10/CSIF | LIF | TNF-alpha |
| IFN-gamma | IL-12 p40 | LIX/GCP2/CXCL5 |
| VEGF-A | IL-1 alpha/IL-1F1 | IL-12 p70 |
| MCP-1/JE/CCL2 | IL-1 beta/IL-1F2 | IL-13 |
| MCP-3/MARC/CCL7 | IL-2 | IL-15 |
| M-CSF/CSF-1 | | |

All specimens were assayed in duplicate following kit manufacturer protocols. Multiplex immunoassays were performed using Luminex 100 IS System (Luminex Corporation, Austin, Tex.). Analyte concentrations were calculated from the standard curves using Bio-Plex Manager 4.1.1 (Bio-Rad Laboratories, Hercules, Calif.).

### Normal Balb/c Mice:

The results demonstrate that in normal mice both oligonucleotides encapsulated in SMARTICLES demonstrated the typical immune response with elevations in IFNγ, IL-12p40, IL-6, MCP-1, MCP-3 and RANTES observed. (FIG. 8). The data show no significant biochemical or statistical differences for these markers between these two groups compared to vehicle controls.

PNT2258 and the scrambled control, showed decreases in the immune markers G-CSF, GM-CSF, IL-12p70, IL-10, IL-1b, IL-1a, a series of other markers and leptin; Increases were noted in IL-12p40 and the chemokines MCP-1, MCP-3 and RANTES.

### WSU-DLCL2 Xenograft Nude Mice:

Testing PNT2258 and the scrambled control encapsulated in SMARTICLES in the same model that antitumor effects were evaluated represents a better system to test whether immune effects contribute to antitumor activity. Importantly, xenografts likely better approximately patients who may be immunosuppressed.

The results show both particles caused elevations in G-CSF, IFNγ, IL-12p40, IL-6, MCP-1, MCP-3 and RANTES observed similar to normal mice, but the magnitude of increase these markers were much greater in xenografts than normal mice and in some cases up to ten-fold higher. (FIG. 9). There were no statistical different differences between in the significant PNT2258 and scrambled, however the trend suggests PNT2258 causes less of an elevation. The results also show decreases in leptin, GM-CSF, IL-12p70, IL-10, IL-1b, IL-1a, and a series of other markers.

### Example 7

### Plasma Protein Biomarkers Post-PNT2258 Administration in Human Patients

### Specimens and Chemicals

Human PBMC specimens were obtained from whole blood of normal healthy donors in EDTA Vacutainer and isolated using Ficoll-Paque density gradients centrifugation were purchased from SeraCare (Milford, Mass.). Frozen preparations were stored in liquid nitrogen until used. Toll-Like Receptor (TLR) agonists: TLR3; poly(I:C)LMW, TLR7; imiquimod, and TLR9; ODN2006, were purchased from InvivoGen (San Diego, Calif.). PNT2258, PNTE (empty liposomes), and scrambled oligonucleotide encapsulated in the same liposome composition as PNT2258 (scrambled control) were used.

### Cell Treatment

Five vials of PBMCs (3 million/mL) were thawed in a water bath at 37° C., washed with 40 mL pre-warmed RPMI1460 (phenol red-free, Gibco) with 10% FBS and 0.1% Pen/Strep (complete medium), adjusted to 5×105 cells/mL with the same medium and plated in 24-well plates at 5×105 cells per well. After one hour equilibration at 37° C., the PBMCs were treated with (a) PNT2258, Scrambled control or empty liposomes control at final concentrations of 7.5, 1.5 and 0.3 μM each. Note: identical dilutions of empty liposomes, were prepared in the complete medium and added to the PBMCs. Untreated control was included. Two identical replicates of set (a) were prepared and exposed to treatment with (b) poly(I:C) (10 μg/mL) and imiquimod (0.25 μg/mL) and (c) ODN2006; (0.5 μg/mL). The concentrations of the TLR agonists were selected to fall within recommended ranges per InvivoGen specifications. Following 24 incubation in a humidified incubator at 37° C., the cells were transferred to labeled Eppendorf tubes and pelleted by centrifugation. Conditioned media was removed to fresh tubes and frozen for multiplex immunoassays. The remaining cells were resuspended in complete medium and tested for viability using MTS assay.

A multiplex immunoassay of 54 analytes was used to identify biomarkers of response or resistance to PNT2258 in patients' plasma. These analytes are shown in the following table.

Human 54-plex

| | | |
|---|---|---|
| beta-NGF/NGFB | IL-1 alpha/IL-1F1 | IL-15 |
| MIP-3 alpha/CCL20 | CD40 ligand/TNFSF5 | IL-1 beta/IL-1F2 |
| IL-16/LCF | PAI-1/Serpin E1** | EOF |
| IL-1RA/IL1RN | IL-17A/CTLA-8 | PDGF-BB |
| ENA-78/CXCL5 | IL-2 | IL-17F/ML-1 |
| RANTES/CCL5** | Eotaxin/CCL11 | IL-4 |
| IL-20 | Resistin/ADSF FGF Basic | IL-5 |
| IP-10/CXCL10 | SAA | Fractalkine |
| IL-6 | I-TAC/CXCL11 | TGF-alpha |
| G-CSF/CSF-3 | IL-7 | Leptin/LEP |
| TGF-beta 1 | GM-CSF/CSF-2 | IL-8/CXCL8 |
| MCP-1/JE/CCL2 | TNF-alpha | GRO alpha/CXCL1 |
| IL-9 | MCP-3/MARC/CCL7 | TNF-beta |
| HGF | IL-10/CSIF | M-CSF/CSF-1 |
| TRAIL/TNFSF10 | IFN-alpha 2 | IL-12/IL-23 p40 |
| MIG/CXCL9 | VEGF-A | IFN-beta |
| IL-12 p70 | MIP-1 alpha/CCL3 | IFN-gamma |
| IL-13 | MIP-1 beta/CCL4 | |

Timepoints were taken before and after (8 and 24 hours) after administration of PNT2258 across successive courses of the drug treatment. A selection of markers results are shown in the table below.

### Selected Results

| | % Change | | % Change | |
|---|---|---|---|---|
| | from | | from | |
| Marker | t0 to t8 | p-value | t8 to t24 | p-value |
| Eotaxin | 43.35339 | 0.01358 | 11.22482 | 0.570851 |
| GM-CSF | 72.20193 | 0.015352 | 47.65702 | 0.022831 |
| IL-13 | 49.31607 | 5.13E−05 | −4.33033 | 0.701455 |
| IL-15 | 29.5966 | 0.049419 | −3.10122 | 0.613218 |
| IL-17A | 40.01748 | 0.005889 | 1.787559 | 0.835988 |
| IL-1α | 99.87154 | 0.009068 | 122.9547 | 0.00856 |
| IL-1β | 44.98659 | 0.000742 | −6.04605 | 0.581557 |
| IL-4 | 62.08945 | 5.4E−05 | 0.712891 | 0.952978 |
| IL-5 | 56.43816 | 3.49E−05 | 0.865636 | 0.940035 |
| IL-6 | 46.3904 | 0.002276 | −0.43276 | 0.972082 |
| IL-8 | 55.72031 | 0.000501 | 9.022011 | 0.297247 |
| IL-9 | 43.2763 | 0.003254 | −3.13797 | 0.726868 |
| IL-10 | 72.20193 | 0.015352 | 47.65702 | 0.022831 |
| Leptin | 102.7819 | 0.005622 | 101.6136 | 0.007946 |
| MCP-1 | 83.84789 | 0.000179 | 12.74902 | 0.412202 |
| MCP-3 | 5.220176 | 0.690909 | −4.72803 | 0.685951 |
| MIP-1α | 66.82023 | 4.4E−07 | 8.193365 | 0.418128 |
| RANTES | 74.2189 | 0.002276 | 9.541819 | 0.690558 |
| TNFα | 55.86931 | 0.000804 | 1.17464 | 0.903976 |
| VEGF-A | 24.25702 | 0.246566 | −1.89025 | 0.942063 |

The spider plot shown in FIG. 10 represents the patient response following PNT2258 treatment. Although many markers show statistical significance (see table above) only key markers are affected by two-fold or greater (annotated with asterisks). PNT2258 induced statistically significant dose-dependent changes in the following markers; Leptin and GM-CSF increased; IL-20, MIP-1a (CCL3), MMP-7, SAA and sCD40L decreased. In addition, increases in IL-RA (interleukin receptor agonist functions to block inflammatory effects of IL-1a and IL-1β) and IP-10 (confirms PNT2258 effect on bone marrow; linked to reducing colony formation) suggest anti-inflammatory and antitumor activity. MIP-1β and MCP-1 signals the recognition of PNT2258 as a nanoparticle and suggests recruitment of innate immune cells.

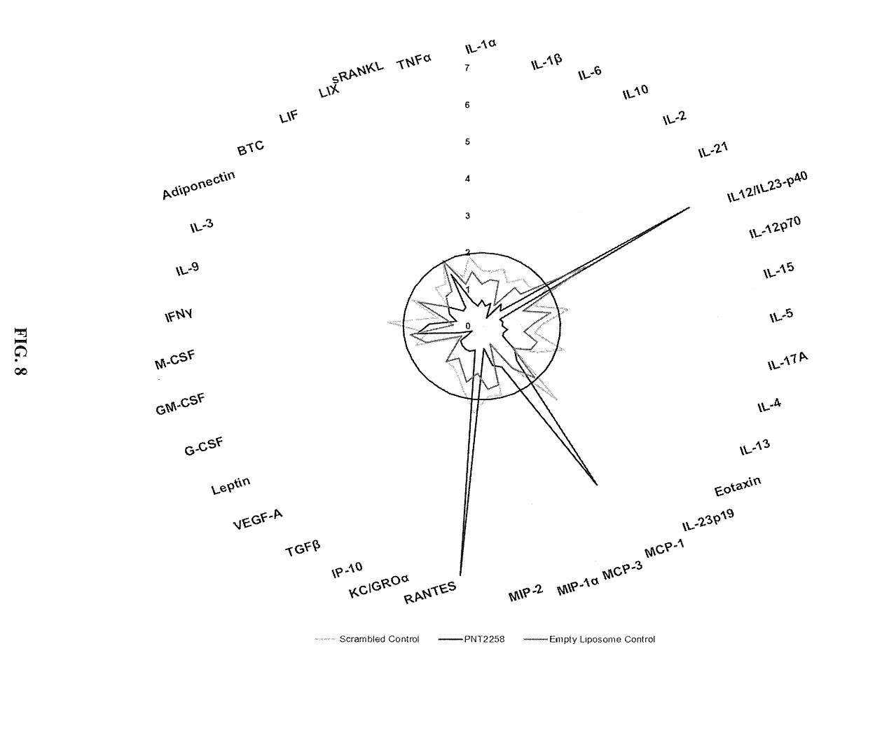

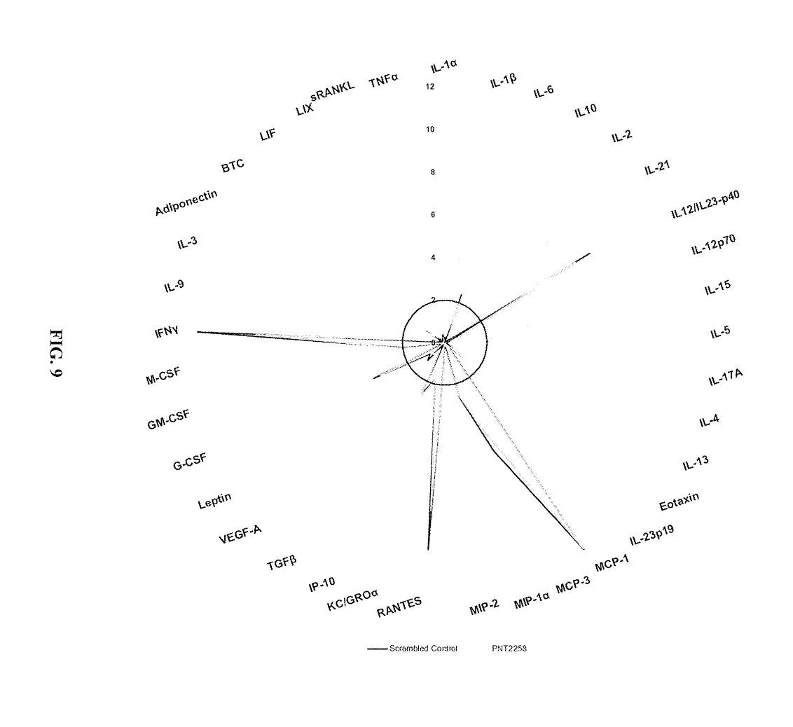

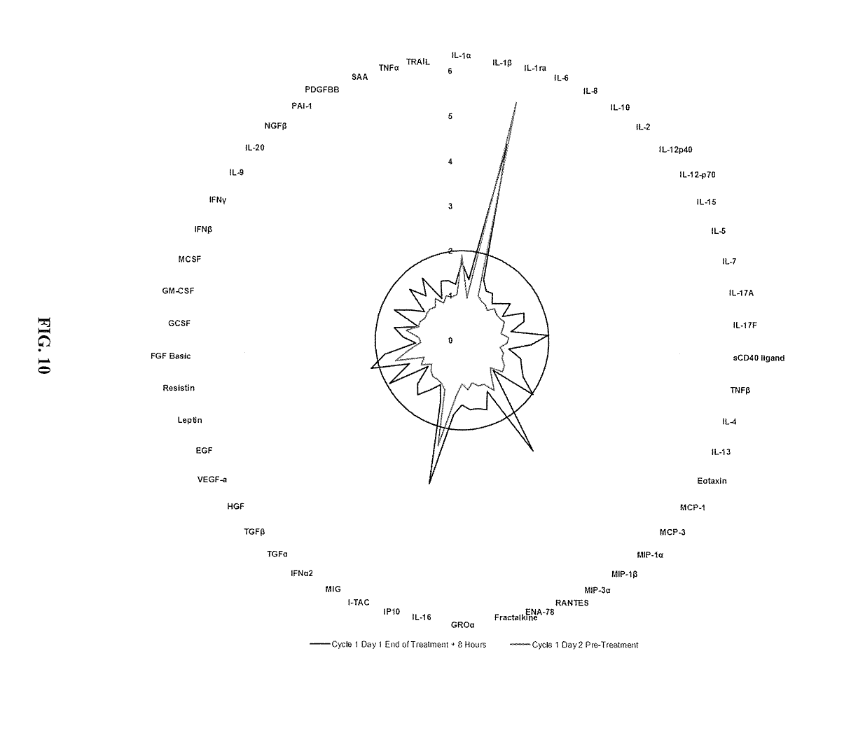

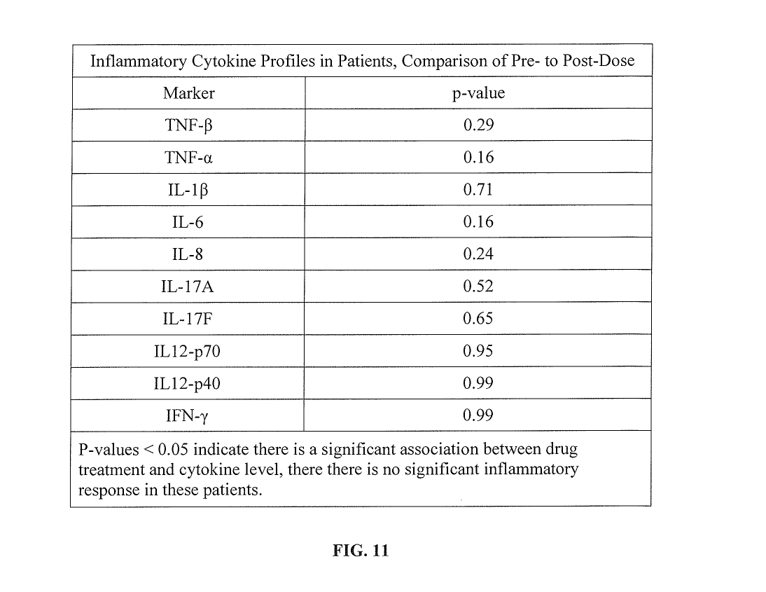

The markers shown in FIGS. 10 and 11 can be linked with BCL2 modulation. The scientific literature contains numerous papers that increased plasma leptin levels are linked to the suppression of cellular BCL2 levels. To our knowledge, there are no papers that report suppression of BCL2 is linked to an increase in plasma leptin. Not to be limited by theory, we propose that leptin and BCL2 work biochemically at cross purposes, i.e., suppressing BCL2 causes a compensatory increase in leptin indicative of therapeutically hitting our target. Increasing dosage was associated with increasing leptin levels.

Similarly, GM-CSF increased in a dose-dependent manner with PNT2258 treatment. GM-CSF functions as a white blood cell growth factor. GM-CSF stimulates stem cells to produce granulocytes (neutrophils, eosinophils, and basophils) and monocytes, the latter which can exit the circulation and migrate into tissue, mature into macrophages and dendritic cells believed to be important to fight cancer. The decrease in the other markers IL-20, platelets (and sCD40L), lymphocytes supports PNT2258's anti-BCL2 effects to promote apoptosis; decrease in SAA supports the lack of an immune response with PNT2258; decrease in MIP-1a and MMP-7 suggests effects on metastases and may support the observation of stable disease seen in several patients.

Immune markers were also included in the panel. Oligonucleotide therapeutics are known to induce immune markers following their administration. Immune stimulation through toll-receptors (TLR-3, TLR-8 and TLR-9) or activation of immune cells (dendritic, NK and T-cells) caused by the preferential uptake of oligonucleotides encapsulated in nanoparticles by macrophage-rich tissues of the RES. Further, preclinical studies have demonstrated that encapsulated oligonucleotides are also known to activate complement factors. These immunomodulatory effects of oligonucleotides are of concern in patients because they may lead to clinical sequelae of fever, chills, or rigors.

The results show that immune markers known to be associated with TLR stimulation in preclinical models and with other oligonucleotide therapeutics were not changed with repeated PNT2258 treatment in patients. The results are shown in FIG. 11.

These results clearly show that in the Phase I study conducted, PNT2258 does not induce an anti-inflammatory response and is not identified as “foreign” by Toll-like Receptors (TLRs). Serum levels of inflammatory cytokines (IL-6 and TNFα) have been reported to be inversely proportional to serum leptin levels. The observation that leptin levels increased following PNT2258 supports the lack of immunostimulation seen following PNT2258 dosing.

The biomarker results are summarized below.

| Parameter | Rats | Monkeys | Patients | Comments |
|---|---|---|---|---|
| Liver | Dose-dependent | Dose-dependent | Increase in one parameter | LFT increase at 150 |
| enzymes | Increases in ALT, | Increases in ALT, | (ALT, AST or Alk Phos) at | mg/m² = DLT. |
| | AST, and Alk Phos | AST, and Alk Phos | the highest dose of 150X; | PNT2258 lipid doses |
| | | | Grade 1 or 2 or two-grade | approach levels in |
| | | | increase from baseline | Intralipid (fat |
| | | | | emulsion for human |
| | | | | use) but dosed at a |
| | | | | rate 2x faster |
| White Blood | Dose-dependent | Dose-dependent | No significant changes in | No significant |
| Cells | Increases | Increases in neutrophils, | WBC noted across dose | clinical toxicity or |
| | (neutrophils, | basophils, lymphocytes | groups | patient management |
| | monocytes) | in 2 cycle toxicology | | issues |
| | | studies in monkeys | | |
| Red Blood | Dose-dependent | Dose-dependent | No significant changes in | No significant |
| Cells | decreases | decreases | WBC noted across dose | clinical toxicity or |
| | | | groups | patient management |
| | | | | issues |
| Platelets | Decreases in platelets | Decrease in platelets | Mild decreases at end of | No significant |
| | at high dose at end of | at high dose at EOI | infusion at lower doses; | clinical toxicity or |
| | infusion (EOI) | | Grade 1 and 2 decreases at | patient management |
| | | | 150 mg/m² and DLT noted in | issues |
| | | | one patient | |
| APTT | Increase in APTT | Increase in APTT | No apparent changes | No significant |
| | time | time | | clinical toxicity or |
| | | | | patient management |
| | | | | issues |
| Lymphocytes | Increases noted | Decreases noted in | Decreases noted at end of | No significant |
| | | exploratory | infusion returning to baseline | clinical toxicity or |
| | | toxicology; Increases | levels at predose of next day | patient management |
| | | noted in 2-cycle | | issues |
| | | toxicology | | |

### Example 9

### Recent In Vitro Results to Further Support Leptin as a Companion Marker for PNT2258

Recent in vitro data with PNT2258 demonstrated even more robust BCL2 reduction after exposure to either PNT2258 or PNT100.

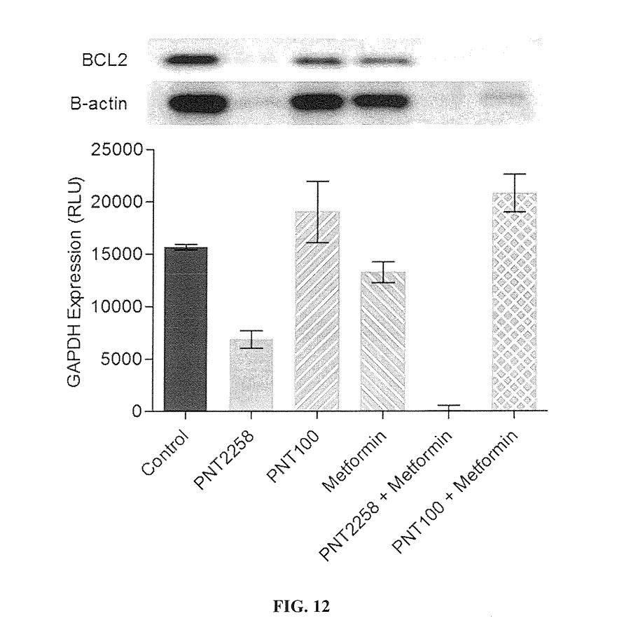

A preliminary study was done to assess whether co-administration of a metabolic-effecting drug, such as the leptin-blocker metformin would have an effect on BCL2 expression in a Pfeiffer human lymphoma cell line. PNT2258, PNT100, PNT2258+metformin (MTF), PNT100+MTF was administered to the Pfeiffer cells in culture. BCL2 expression levels and b-actin levels were monitored by Western blot, as well as the levels of GAPDH in the culture medium. B-actin and GAPDH may be taken as markers of loss of cell function (e.g., after BCL2 down-regulation—caused apoptosis initiation. After 6 days in culture, PNT2258+metformin or PNT100+metformin results in synergy for BCL2, and b-actin. A synergistic reduction of GAPDH was seen with the PNT2258+MTF treatment. (See FIG. 12.)

These reductions support the hypothesis that modulation of insulin and leptin signaling subverts the resistance pathway of cancer cells to PNT2258 treatment. The extent of BCL2 protein knockdown and the viability of cells can be dialed down depending on the in vitro conditions applied. These data suggest that in a dynamic environment (i.e. samples from tumor xenografts or patient PBMC analyzed ex vivo) the snapshot measured only represents the viable cells rather than all cells that can be analyzed in an in vitro setting such as in the Western Blots above. Note also that while both PNT100 and PNT2258 reduce BCL2 protein expression, PNT2258 facilitates better nuclear uptake (and therefore effect) than naked PNT100 given the effective delivery to cells and nucleus with a liposome-encapsulated oligo. Note also that there is a clear synergy of BCL2 knockdown with metformin (a blocker of leptin and MAPK) and corresponding cell death (see FIG. 12) that highlights that specific pathways are activated in response to PNT100 or PNT2258's BCL2 specific knockdown. Increased leptin concentration levels following PNT2258 administration in patients suggests its utility as a “biomarker” of PNT2258-induced BCL2 knockdown.

As PNT2258 is designed to decrease cellular BCL2 levels, these data support the proposal that the drug is in fact reaching and acting on the intended gene target as designed. The findings are very provocative and strongly support the contention that PNT2258 is acting to suppress BCL2 transcription and production. The relevance of these markers: (1) highlight the specific mechanism of action of PNT2258, (2) serve as biomarkers to PNT2258 administration (3) identify patients that may be responsive to PNT2258 treatment and (4) guide the identification of potential therapies to work synergistically with PNT2258. There are clear differences between preclinical and patient's biomarker results. The increase in leptin and GM-CSF observed in patients could not be predicted by the preclinical results.

### Example 10

### Experimental Design of Single Arm Proof of Concept Study in Human Patients with Refractory or Relapsed Non-Hodgkin's Lymphoma

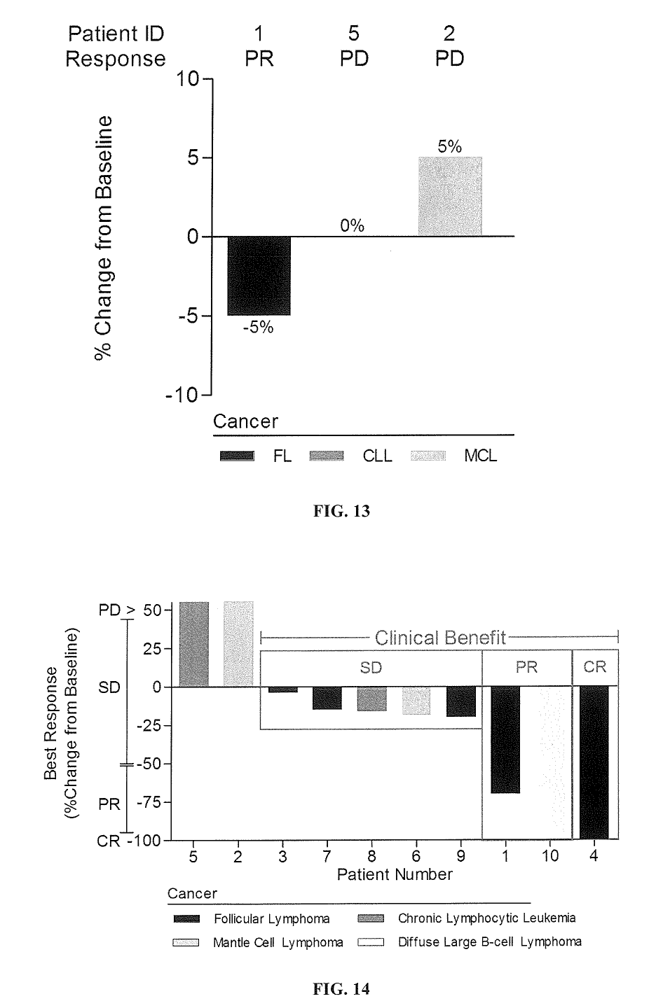

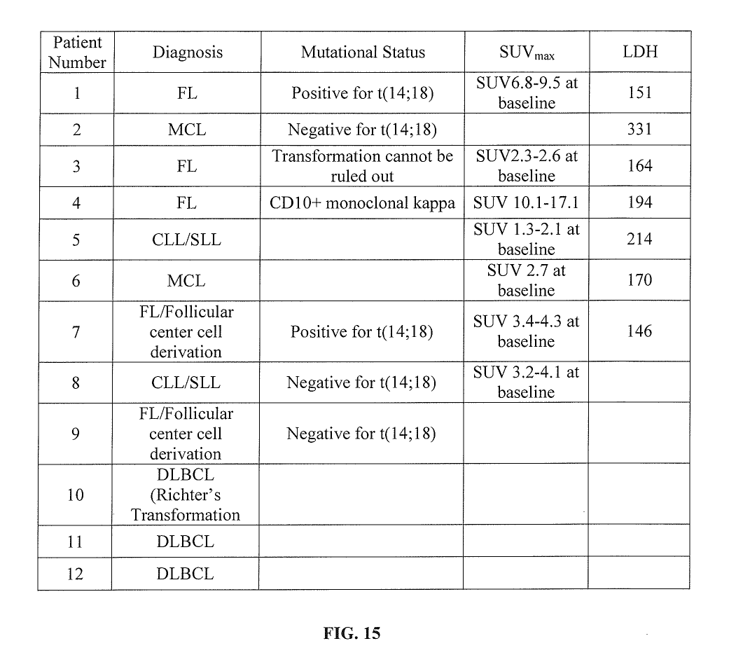

An open-label, single-arm, Phase 2 study of PNT2258 in human patients with relapsed or refractory non-Hodgkin's lymphoma. Patients (patient demographics, FIG. 2; diagnosis and measurement of molecular characteristics FIG. 15; response to treatment, FIG. 14; Ki-67 modulation, FIG. 13) received PNT2258 at 120 mg/m² as an intravenous infusion over 3 hours once daily for 5 consecutive days (Days 1-5) of a 21-day cycle (3 weeks). Treatment may continue unless there is disease progression or the occurrence of unacceptable toxicity for a total of 6 cycles of therapy.

Inclusion criteria for the study included, but was not limited to: morphologically confirmed diagnosis of non-Hodgkin's lymphoma, acceptable Eastern Cooperative Oncology Group (ECOG) performance status and hematological, hepatic and renal function, at least a single measurable tumor mass (long axis >1.5 cm), an FDG-PET positive baseline scan defined as “focal or diffuse FDG uptake above background in a location incompatible with normal anatomy or physiology, without a specific standardized uptake value cutoff,” disease that has relapsed after administration of primary therapy, have discontinued all prior anti-cancer therapies for at least 21 days. Relapsed disease after administration of primary therapy (e.g. rituximab and CHOP, EPOCH, bendamustine or similar chemotherapy or subsequent salvage regimen) is defined as progression after a complete response to therapy or radiographic evidence of active disease after a partial response or stable disease. Have received three or fewer complete courses of systemic cytotoxic regimens. Note: Rituximab (alone or in combination with cytotoxic chemotherapy) is not considered a cytotoxic regimen.

A maintenance phase extension for subjects that have completed the initial 6 cycles of study treatment allowed during participation (termed the induction phase) Subjects who have continuing evidence of clinical benefit (stable disease or better) at the time that they complete the induction phase may be considered for participation in the maintenance phase of treatment. Patients may continue participation in the maintenance phase of PNT2258-02 until such time as they experience disease progression, intolerable toxicity, request for voluntary withdrawal or if, in the opinion of the investigative physician, subjects are no longer benefiting from exposure to PNT2258. Patients will receive PNT2258 as an IV infusion over 2 hours, once daily for 2 consecutive days (Days 1-2) of every 28-day cycle (4 weeks). The dose of PNT2258 used for the maintenance phase of the study is 100 mg/m² based upon each subject's calculated body surface area with the maximum calculated BSA to not exceed 2.0

FIG. 2 provides the patient information and assignment into the single arm proof of concept study, and also shows the number of patients having a particular cancer type by study.

### V. Other Embodiments

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims.

All references cited herein, are incorporated herein by reference in their entirety.

What is claimed is:

1. A method of determining modulation of BCL2 transcription, translation, or expression after administration of a test compound for the treatment of a BCL2 mediated cancer in a subject having cancer comprising:
administering the test compound;
obtaining a biological sample or radiological image from the subject, subsequent to the administration of said test compound;
detecting a measurable level of one or more of a biomarker in the biological sample, wherein the biomarker is selected from the group consisting of Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof.

2. The method of claim 1, wherein the test compound is an oligomer that hybridizes under physiological conditions to an oligonucleotide sequence selected from SEQ ID NO: 1249 or 1254 or the complements thereof.

3. The method of claim 2, wherein the oligomer is selected from the group consisting of SEQ ID NOs:1250, 1251, 1252, 1253, 1267-1477 or the complements thereof.

4. The method of claim 3, wherein the oligomer is selected from the group consisting of SEQ ID NOs:1250, 1251, 1289-1358 or the complements thereof.

5. The method of any one of claim 4, wherein the oligomer comprises SEQ ID NO:1250 or 1251.

6. The method of claim 6, wherein the oligomer comprises SEQ ID NO:1251.

7. The method of any one of claims 1-6, wherein the oligomer is administered in a liposome formulation.

8. The method of claim 7, wherein the liposome formulation is an amphoteric liposome formulation.

9. The method of claim 8, wherein the amphoteric liposome formulation comprises one or more amphoteric lipids.

10. The method of claim 9, wherein the amphoteric liposome formulation is formed from a lipid phase comprising a mixture of lipid components with amphoteric properties.

11. The method of claim 10 wherein the mixture of lipid components are selected from the group consisting of (i) a stable cationic lipid and a chargeable anionic lipid, (ii) a chargeable cationic lipid and chargeable anionic lipid and (iii) a stable anionic lipid and a chargeable cationic lipid.

12. The method of claim 11, wherein the lipid components comprise one or more anionic lipids selected from the group consisting of DOGSucc, POGSucc, DMGSucc, DPGSucc, DGSucc, DMPS, DPPS, DOPS, POPS, DMPG, DPPG, DOPG, POPG, DMPA, DPPA, DOPA, POPA, CHEMS and Cet-P.

13. The method of claim 11, wherein the lipid components comprise one or more cationic lipids selected from the group consisting of DMTAP, DPTAP, DOTAP, DC-Chol, MoChol, HisChol, DPIM, CHIM, DORIE, DDAB, DAC-Chol, TC-Choi, DOTMA, DOGS, (C18)2Gly+N,N-dioctadecylamido-glycine, CTAP, CPyC, DODAP and DOEPC.

14. The method of any one of claims 10-13, wherein the lipid phase further comprises neutral lipids.

15. The method of claim 14, wherein the neutral lipids are selected from sterols and derivatives thereof, neutral phospholipids, and combinations thereof.

16. The method of claim 15, wherein the neutral phospholipids are phosphatidylcholines, sphingomyelins, phosphoethanolamines, or mixtures thereof.

17. The method of claim 16, wherein the phosphatidylcholines are selected from the group consisting of POPC, OPPC, natural or hydrogenated soy bean PC, natural or hydrogenated egg PC, DMPC, DPPC, DOPC or derivatives thereof; and the phosphatidylethanolamines are selected from the group consisting of DOPE, DMPE, DPPE, or derivatives thereof.

18. The method of claim 17, wherein the amphoteric liposome comprises DOPE, POPC, CHEMS and MoChol.

19. The method of claim 18, wherein the molar ratio of POPC/DOPE/MoChol/CHEMS is about 6/24/47/23.

20. The method of any one of claims 1-19, wherein one or more biomarker is a protein, and wherein the detecting further comprises assaying the measurable level of protein expression in the biological sample using mass spectroscopy, an immunoassay or a combination thereof.

21. The method of any one of claims 1-20, wherein the biological sample is selected from blood, plasma, serum, normal tissue, PBMCs, tumor tissue, urine, or buccal swab.

22. The method of any one of claims 1-21, wherein a first biological sample is taken from the subject prior to administration of the test compound.

23. The method of claim 22, wherein the biological sample obtained subsequent to the administration of the test compound is compared to the first biological sample.

24. A method of treating a BCL2 mediated cancer in a subject, comprising:
administering a test compound;
obtaining one or more of a biological sample or radiological image from the subject, subsequent to the administration of said test compound;
determining a presence of one or more of a biomarker in the biological sample or said image, wherein the biomarker is selected from the group consisting of Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, clinical or imaging parameters including FDG-PET uptake (standard uptake value, SUV) and CT imaging, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof;
and comparing the presence of the biomarker with a measurable level or expression of the biomarker.

25. The method of claim 24, further comprising modifying the treatment of the BCL2 mediated cancer using the comparison between the biomarker presence in the biological sample and the biologically relevant level or expression.

26. The method of claim 24, further comprising collecting a first biological sample from the subject prior to the test compound administration.

27. The method of claim 26, wherein the biomarker is leptin and the presence of leptin in the first biological sample initiates a treatment or modification of the BCL2 mediated cancer.

28. The method of any one of claims 24-27, wherein an overall survival rate of the patient is improved.

29. The method of any one of claims 24-28, wherein a progression-free of the patient is improved.

30. The method of any one of claims 24-29, wherein a tumor size is decreased in the patient.

31. The method of any one of claims 24-30, wherein a tumor metabolism of radioloabeled glucose is decreased.

32. The method of claim 31, wherein the tumor metabolism is measured by FDG-PET.

33. The method of any one of claims 24-31, wherein a quality of life of a patient is increased.

34. The method of any one of claims 24-33, wherein an ECOG performance of a patient status is improved.

35. The method of any one of claims 24-34, wherein a Cheson criteria of a patient is improved.

36. A method of inhibiting expression of BCL2 in a subject in need thereof, comprising
administering a test compound;
wherein the inhibiting of the expression of BCL2 in a subject, modulates the expression of one or more of the following biomarkers Ki-67, BCL2, CD10, CD5, CD38, BCL6, MUM1, TP53, ZAP 70, immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof.

37. A kit for determining the modulation of BCL2 transcription, translation, or expression after administration of a test compound for the treatment of a BCL2 mediated cancer in a subject having cancer comprising:
probes for detecting the levels of one or more of a biomarker in the biological sample, wherein the biomarker is selected from the group consisting of;
Ki-67, BCL2, CD10, CD5, CD38, BCL6, MUM1, TP53, ZAP 70,
immunohistochemistry including immunohistochemistry panels, flow cytometry analyses, gene expression panels, gene aberration panels, genetic deletions, translocations, amplifications and mutations, cytogenetics for chromosomal rearrangements of BCL2, e.g. t(14; 18), t(14;18)(q32;q21.3) and rarely to IG light chain (IGK, IGL) loci as t(2;18)(p11;q21.3) or t(18;22)(q21.3;q11) or chromosomal rearrangements in CMYC or other genes, proteins, and/or factors implicated in driving the transcription and/or overexpression of BCL2, phosphorylated BCL2, active capsase-3, PARP, cytochrome c, LDH, absence of B-symptoms, AKT signaling pathway markers, BCL2 family members such as BAX, lymphocyte counts, platelet counts, leptin, IL-1ra, IL-17a, MCP-1, MIP-1β, and IP10 or combinations thereof.

38. A method of identifying a patient with a response profile to a treatment with a test compound using a biomarker selected from an immunohistochemical analyses; flow cytometric; cytogenetic or clinical analyses; levels of BCL2, CD10, Ki-67, MYC, t(14;18), TP53, CD38, ZAP 70, or LDH; patient age, MYC aberration, genetic deletions, translocations, amplifications, mutations or clinical or imaging parameters including PET SUV levels, CT imaging, R-IPI, FLIPI, Rai criteria, performance status, presence or absence of B-symptoms, or age of the subject.

39. The method of claim 38, wherein the PET SUV is greater than or equal to 5.

40. The method of claim 38, wherein the patient age is greater than or equal to 60.

41. The method of claim 38, wherein Ki-67 is positive in the patient.

42. The method of claim 38, wherein BCL2 is positive in the patient.

43. The method of claim 38, wherein the translocation is a t(14,18) or BCL2 translocation in the patient.

44. The method of claim 38, wherein the MYC aberration is present in the patient.

45. The method of claim 38, wherein the CD10 is positive in the patient.

46. The method of claim 38, wherein the R-IPI is greater than or equal to 3.

47. The method of claim 38, where the TP53 is positive in the patient.

48. The method of claim 38, wherein the CD38 is positive in the patient.
